SEQUENCE LISTING (38 sequences)

SEQ ID NO: 1 - DNA, 1284 bp, Coleus forskohlii
atgaaggtggaaagatttagcaggaaactcataaaaccgcacaccccaacccccgaaaatctgaagaagtataagctttctcttttagacaaatgcttggggcacgataattttgctattgttctgttttacgaatcgaaaccaagaaacaagagtgagttggaagaatcactggaaaaagtcctggtggatttctaccctcttgctggaagacacaccatgaatgatcacatagttgattgcagtgatgtgggcgccgtgtttgtcgaagcagaagctctagacgtcgagctgacgatggatgagctcgttaagaacatggaggcccagactatccatcatctccttcccaatcaatatttctcagctgatgctccaaatccactcttgtcaatccaggtgacacatttcccatctggtggcctagcaattggcatcgccgtttctcacgctgtcttcgatggtttttcgctgggggtgttcgtcgccgcctggtcgaaggccaccatgaacccagatcggaagatcaaaatcacaccgtctttcgatcttccctcgttgcttccttacaaggatgacaattttggattaactgccgccgaaattgtcagccagagtgaagatattgttgttaagaggtttattttcgggaaggaggcaataacgaggttgagatcaaagctaagcccaaatcgcaatgggaaaaagatctctcgtgttcgagtggtgtgtgccgttatagtgaaagccttgatgggcctggaacgcgccaaacatggcaaaacaagagatttcttgatcactcaatcgattaacatgcgcgagagaacaaaagcacctctacagaagcatgcttgtgggaatctggcagtcttgtcgtgcacgcgacgtgtagaagccgaggagatgatggagctgcagaacttggttaatctgatcggagattctacagagaaggacatagctgattttgctgaattgctatctcctgatcaagttgggcgtgatatcatcatcaagatgatgaagtctttcatgcaattcttggataatgacatttattctgtatgcttcactgattggagtaagtttgaattttacgaagctgattttgggttcgggaagcctgtttggatggccgctggaccgcaacgcccaatcattagcaccgctattctcatgagcgacagagagggtgatggaattgaggcatggctgcatctcaacaaaaatgacatgcttatcttcgagcaagatgaagaaatcaaattatttactacctaa

SEQ ID NO: 2 - DNA, 1284 bp, Coleus forskohlii
atgaaggtggaaagatttagcaggaaactcataaaaccgcacaccccaacccccgaaaatctcaagaattataagctttctcttttagataaatgcttggtgcaggataattttgctgttgtgctgttttacgaatcgaaaccaagaaacaagagtgagttggaagaatcactagaaaaagtccttgtcgatttctaccctcttgctggaagatacaccatgaatgatcacatagttgattgcagtgatgagggcgccgtgtttgtcgaagctgaagctctggatgctgagctgacgatggatcagctcgtcaagaatatggaggcccagactatccatcatctccttccggatcaatatttcccagctgatgctccaattccactcctctcaatccaggtcacacatttcccatttgggggcctggcaattgccatcgtcgtttctcacgctgtattcgaaggtttttcactcggggtgttcgtcgccgcctggtcgaaggccaccattaatccagatgtgaagatcgaaatcaccccgtctttcgatcttccctcattgcttccatacaaggatgacgatttcggattaactgactgtgaaattattaacctgtgtgaggatattgttgttaagaggtttatgtttgggaaggaggctataacgaggttgagatcaagactaagcccaaatcacaatgggaaaacgatctctcgtgttcgagtggtgtgtgccgttatagtgaaagccttgatgggcctggaacgcgccaaacatggcaaaacaagagatttcttgatcactcaatcgattaacatgcgcgagagaacaaaagcacctctacagaagcatgcttgtgggaatctggcagtcttgtcgtgcacgcgacgtgtagaagccgaggagatgatggagctgcagaacttggttaatctgatcggagaatctactgagaaggacatagctcattattctgaattgctgtctcataatcaatttgggcgtgatatcatcgtcaacgtgatgaaatctctcatgcaattcttggatcctgacatttattctgtatgcttcactgattggagtaagtttagattctacgaagctgattttgggttcgggaagcctgtttggacggccgttggaccgcaacgcccaatcattaccaccgctattctcatgaacaacagagagggtgatggaattgaggcatggctgcatctcaacaaaaatgacatgcttatcttcgagcaagatgaagaaatcaaattatttactacctaa

SEQ ID NO: 3 - DNA, 1278 bp, Artificial sequence
  Chimeric sequence comprising sequences from two ACTs from Coleusforskohlii
atgaaggtggaaagatttagcaggaaattcataaaaccacacaccccgacccccgaaaatctgaagaagtataagctttctcttttagacaaatgcttggggcacgataattttgctattgttctgttttacgaatcgaaaccaagaaacaagagtgagttggaagaatcactggaaaaagtcctggtggatttctaccctcttgctggaagacacaccatgaatgatcacatagttgattgcagtgatgtgggcgccgtgtttgtcgaagcagaagctctagacgtcgagctgacgatggatgagctcgttaagaacatggaggcccagactatccatcatctccttcccaatcaatatttctcagctgatgctccaaatccactcttgtcaatccaggtgacacatttcccatctggtggcctagcaattggcatcgccgtttctcacgctgtcttcgatggtttttcgctgggggtgttcgtcgccgcctggtcgaaggccaccatgaacccagatcggaagatcaaaatcacaccgtctttcgatcttccctcgttgcttccttacaaggatgacaattttggattaactgccgccgaaattgtcagccagagtgaagatattgttgttaagaggtttattttcgggaaggaggcaataacgaggttgagatcaaagctaagcccaaatcgcaatgggaaaaagatctctcgtgttcgagtggtgtgtgccgttatagtgaaagccttgatgggcctggaacgcgccaaaacaagagatttcatgatctgtcaagggatcaacatgcgtgagagaacaaaggcacctctacagaagcatgcttgtgggaatctggcggtctcgtcttacacgcgacgtgtagccgcagcagaagccgaggagctgcagagtttggtaaatctaataggtgattctattgagaagagcatagctgattatgctgatattctttcttctgatcaagatgggcgtcatatcatcagcacgatgatgaaatctttcatgcaattcgcggctcctgacataaaagctatatccttcactgattggagtaagtttggattctaccaagttgattttgggttcgggaagcctgtttggacgggtgttcggcccgaacgcccaatctttagcgccgctattctcatgagcaacagagagggtgatggaattgaggcatggcttcatcttgacaaaaatgacatgcttatcttcgagcaagatgaagaaatcaaattattaattactacctaa

SEQ ID NO: 4 - DNA, 1281 bp, Artificial sequence
  Chimeric sequence comprising sequences from Coleus forskohlii
atgaaggtggaaagatttagcaggaaattcataaaaccacacaccccgacccccgaaaatctgaagaagtataagctttctcttttagacaaatgcttggggcacgataattttgctattgttctgttttacgaatcgaaaccaagaaacaagagtgagttggaagaatcactagaaaaagtccttgtcgatttctaccctcttgctggaagatacaccatgaatgatcacatagttgattgcagtgatgagggcgccgtgtttgtcgaagccgaagctccaaacgtcgagctgacggtggatcaactcgtcaagaacatggaggcccagactatccatgatttccttcccgatcaatatttcccagctgatgctccaaatccactcctctcgatccaggtcacgcatttcccatgtggtggcttggcgattggcatcgttgtttctcacgctgtcttcgatggtttttcgctgggggtgttccttgccgcctggtcgaaggccaccatgaacccagagaggaagatcgaaatcaccccgtctttcgatcttccttcgttgcttccatacaaggatgaaagtttcggattaaatttcagcgaaattgtcaaggctgaaaatattgttgtgaagaggcttaatttcgggaaggaggctataacgaggttgagatcaagactaagcccaaatcacaatgggaaaacgatctctcgtgttcgagttgtgtgtgcccttatagtgaaagccttgatgggcctggaactcgccaagcatggcaaaacaagagatttcatgatctctcaagggattaacatgcgcgagagaacaaaagcacctctacacaagcatgcttgtgggaatctagcaatcttgtcgtgcacgcgacgtgtagaagccgaggagatgatggacctgcaaaacttggttaatctgatcggagaatctactgagaaggacatagctcattattctgaattgctgtctcataatcaatttgggcgtgatatcatcgtcaacgtgatgaaatctctcatgcaattcttggatcctgacatttattctgtatgcttcactgattggagtaagtttagattctacgaagctgattttgggttcgggaagcctgtttggacggccgttggaccgcaacgcccaatcattaccaccgctattctcatgaacaacagagagggtgatggaattgaggcatggctgcatctcaacaaaaatgacatgcttatcttcgagcaagatgaagaaatcaaattatttactacctaa

SEQ ID NO: 5 - DNA, 1302 bp, Coleus forskohlii
atgaaggtggaaagatttagcaggaaattaataaaaccagtcaccccaactccacaaaacctcaagaacttcaacctttcgattttagataaatgtcttccgccgattaaatttggtgttgttttgttttatgaatctaaaccaggaaataagagcgagttggaagaatcactaaaagaagttctggtcgacttctaccctcttgctggaagacacaccatcaatgatcccgtggttgattgcagtgatcagggcgccgtgttcgtcgaagccgaagctctagacaccgagctgaccatggatcagctggtgttgaataagatggagatccagaaagtcgatcaattccttcccgatgaatgcatccaagctgatgctccgaatccactcctgtggatccaggtgacacatttcccatcgggtgggctggcgatcggcgttgcggtttctcactctgtcttcgattccttctcgctgggggtgttcatcgccgcctggtccaaggccacaatgaatccaggtaggatgatcgaaatcaccccgtctttcgatgttccctcgttgcttccatgcaaggatcacgatttcgagatagctctcaatgaaattacggatcagggtgaaagcttcgttgttaagaggctcgtgttcggtaaggaggctataacgaggttgagatcaaaactaagcctaaatcaagatggtaaaacgatctctcgtgttcgtgttgtgggtgccgttctagtgaaagccctgatcggcctggaatgtggcaaacacggcagaagaaaagatctcgtgatctctctgccggttaacatgcgtgagagaacaaacacacctctacagaatccaaagcatgcttgcgggaatctggcggtcatttcgctcacgcgatgcgtagctgcagcagaagctgaggagatggggctgcaggagttggtaaatctacttggagatgcgattggaaaagcgatagctgatcatgctgaaatgttgtctcctaatcaagaagggtgtgacatcattattaatgatttcaagaactttttaacattattcgggactcctaacacaaatattattactcttactgattggagtaagtttggattctacgaagctgattttgggtttgggaagcctgtttggaccagcagcggacagcaatccctgagcgttaccaccattgtgctcatgaacaacaaagagggcgatggaatcgaggcatggctgcatctcaacaaaaatgacatgcttttcttcgagcaagatgaagaaatcaaattatttactacctaa

SEQ ID NO: 6 - protein, 427 aa, Coleus forskohlii
MKVERFSRKLIKPHTPTPENLKKYKLSLLDKCLGHDNFAIVLFYESKPRNKSELEESLEKVLVDFYPLAGRHTMNDHIVDCSDVGAVFVEAEALDVELTMDELVKNMEAQTIHHLLPNQYFSADAPNPLLSIQVTHFPSGGLAIGIAVSHAVFDGFSLGVFVAAWSKATMNPDRKIKITPSFDLPSLLPYKDDNFGLTAAEIVSQSEDIVVKRFIFGKEAITRLRSKLSPNRNGKKISRVRVVCAVIVKALMGLERAKHGKTRDFLITQSINMRERTKAPLQKHACGNLAVLSCTRRVEAEEMMELQNLVNLIGDSTEKDIADFAELLSPDQVGRDIIIKMMKSFMQFLDNDIYSVCFTDWSKFEFYEADFGFGKPVWMAAGPQRPIISTAILMSDREGDGIEAWLHLNKNDMLIFEQDEEIKLFTT

SEQ ID NO: 7 - protein, 427 aa, Coleus forskohlii
MKVERFSRKLIKPHTPTPENLKNYKLSLLDKCLVQDNFAVVLFYESKPRNKSELEESLEKVLVDFYPLAGRYTMNDHIVDCSDEGAVFVEAEALDAELTMDQLVKNMEAQTIHHLLPDQYFPADAPIPLLSIQVTHFPFGGLAIAIVVSHAVFEGFSLGVFVAAWSKATINPDVKIEITPSFDLPSLLPYKDDDFGLTDCEIINLCEDIVVKRFMFGKEAITRLRSRLSPNHNGKTISRVRVVCAVIVKALMGLERAKHGKTRDFLITQSINMRERTKAPLQKHACGNLAVLSCTRRVEAEEMMELQNLVNLIGESTEKDIAHYSELLSHNQFGRDIIVNVMKSLMQFLDPDIYSVCFTDWSKFRFYEADFGFGKPVWTAVGPQRPIITTAILMNNREGDGIEAWLHLNKNDMLIFEQDEEIKLFTT

SEQ ID NO: 8 - protein, 425 aa, Artificial sequence
  Chimeric sequence comprising sequences from two ACTs from Coleus forskohlii
MKVERFSRKFIKPHTPTPENLKKYKLSLLDKCLGHDNFAIVLFYESKPRNKSELEESLEKVLVDFYPLAGRHTMNDHIVDCSDVGAVFVEAEALDVELTMDELVKNMEAQTIHHLLPNQYFSADAPNPLLSIQVTHFPSGGLAIGIAVSHAVFDGFSLGVFVAAWSKATMNPDRKIKITPSFDLPSLLPYKDDNFGLTAAEIVSQSEDIVVKRFIFGKEAITRLRSKLSPNRNGKKISRVRVVCAVIVKALMGLERAKTRDFMICQGINMRERTKAPLQKHACGNLAVSSYTRRVAAAEAEELQSLVNLIGDSIEKSIADYADILSSDQDGRHIISTMMKSFMQFAAPDIKAISFTDWSKFGFYQVDFGFGKPVWTGVRPERPIFSAAILMSNREGDGIEAWLHLDKNDMLIFEQDEEIKLLITT

SEQ ID NO: 9 - protein, 426 aa, Artificial sequence
  Chimeric sequence comprising sequences from Coleus forskohlii
MKVERFSRKFIKPHTPTPENLKKYKLSLLDKCLGHDNFAIVLFYESKPRNKSELEESLEKVLVDFYPLAGRYTMNDHIVDCSDEGAVFVEAEAPNVELTVDQLVKNMEAQTIHDFLPDQYFPADAPNPLLSIQVTHFPCGGLAIGIVVSHAVFDGFSLGVFLAAWSKATMNPERKIEITPSFDLPSLLPYKDESFGLNFSEIVKAENIVVKRLNFGKEAITRLRSRLSPNHNGKTISRVRVVCALIVKALMGLELAKHGKTRDFMISQGINMRERTKAPLHKHACGNLAILSCTRRVEAEEMMDLQNLVNLIGESTEKDIAHYSELLSHNQFGRDIIVNVMKSLMQFLDPDIYSVCFTDWSKFRFYEADFGFGKPVWTAVGPQRPIITTAILMNNREGDGIEAWLHLNKNDMLIFEQDEEIKLFTT

SEQ ID NO: 10 - protein, 433 aa, Coleus forskohlii
MKVERFSRKLIKPVTPTPQNLKNFNLSILDKCLPPIKFGVVLFYESKPGNKSELEESLKEVLVDFYPLAGRHTINDPVVDCSDQGAVFVEAEALDTELTMDQLVLNKMEIQKVDQFLPDECIQADAPNPLLWIQVTHFPSGGLAIGVAVSHSVFDSFSLGVFIAAWSKATMNPGRMIEITPSFDVPSLLPCKDHDFEIALNEITDQGESFVVKRLVFGKEAITRLRSKLSLNQDGKTISRVRVVGAVLVKALIGLECGKHGRRKDLVISLPVNMRERTNTPLQNPKHACGNLAVISLTRCVAAAEAEEMGLQELVNLLGDAIGKAIADHAEMLSPNQEGCDIIINDFKNFLTLFGTPNTNIITLTDWSKFGFYEADFGFGKPVWTSSGQQSLSVTTIVLMNNKEGDGIEAWLHLNKNDMLFFEQDEEIKLFTT

SEQ ID NO: 11 - DNA, 1284 bp, Artificial sequence
  Sequence codon optimised for expression in S. cerevisiae
atgaaggtcgaaagattctccagaaagttgattaagccacatactccaactccagaaaacttgaagaagtacaagttgtccttgttggataagtgcttgggtcatgataatttcgccatcgttttgttctacgaatccaagccaagaaacaagtccgaattggaagaatccttggaaaaggttttggttgacttttatccattggctggtagatacaccatgaacgatcatatagttgattgctctgatgttggtgccgtttttgttgaagctgaagctttggatgttgaattgaccatggatgaattggtcaagaacatggaagctcaaaccatccatcatttgttgccaaatcaatacttctctgctgatgctccaaatcctttgttgtctattcaagttacccatttcccatctggtggtttggctattggtattgctgtttctcatgctgttttcgacggtttttctttgggtgttttcgttgctgcttggtctaaagctactatgaatccagatagaaagatcaagatcaccccatcttttgacttgccatctttgttaccatacaaggatgataacttcggtttgactgctgctgaaatcgtttctcaatctgaagatatcgtcgtcaagagattcatcttcggtaaagaagctatcactagattgagatccaagttgtctccaaacagaaacggtaagaagatctccagagttagagttgtttgtgccgttatagttaaggctttgatgggtttggaaagagctaaacacggtaagactagagatttcttgatcacccaatccatcaacatgagagaaagaacaaaagccccattgcaaaaacatgcttgtggtaatttggctgttttgtcttgtaccagaagagttgaagccgaagaaatgatggaattgcaaaacttggttaacttgatcggtgactctaccgaaaaggatattgctgatttcgccgaattattgtccccagatcaagttggtagagacatcattatcaagatgatgaagtccttcatgcaattcttggacaacgacatctactctgtttgtttcactgattggtctaagttcgaattctacgaagccgattttggttttggtaaaccagtttggatggctgctggtccacaaagaccaattatttctactgccatcttgatgtccgatagagaaggtgatggtattgaagcttggttgcatttgaacaagaacgacatgttgatcttcgaacaagacgaagaaatcaagttgttcaccacctga

SEQ ID NO: 12 - DNA, 1284 bp, Artificial sequence
  Sequence codon optimised for expression in S. cerevisiae
atgaaggtcgaaagattctccagaaagttgattaagccacatactccaactccagaaaacttgaagaactacaagttgtccttgttggataagtgcttggtccaagataatttcgccgttgttttgttctacgaatccaagccaagaaacaagtccgaattggaagaatccttggaaaaggttttggttgacttttatccattggctggtagatacaccatgaacgatcatatagttgattgctctgatgaaggtgccgtttttgttgaagctgaagctttggatgctgaattgactatggatcaattggtcaagaacatggaagcccaaaccattcatcatttgttgccagatcaatactttccagctgatgctccaattcctttgttgtctattcaagttacccatttcccatttggtggtttggctattgctatcgttgtttctcatgctgttttcgacggtttttctttgggtgttttcgttgctgcttggtctaaagctactattaacccagatgtcaagatcgaaattaccccatcttttgacttgccatccttgttgccatacaaggacgatgattttggtttgaccgattgcgaaatcatcaacttgtgtgaagatatcgtcgtcaagagattcatgttcggtaaagaagctatcaccagattgagatctagattgtctccaaaccataacggtaagaccatctctagagttagagttgtttgtgccgttatcgttaaggctttgatgggtttggaaagagctaaacacggtaaaaccagagatttcttgatcacccaatccatcaacatgagagaaagaacaaaagccccattgcaaaaacatgcttgtggtaatttggctgttttgtcttgtaccagaagagttgaagccgaagaaatgatggaattgcaaaacttggttaacttgatcggtgaatccaccgaaaaggatattgctcactactccgaattattgtcccacaatcaattcggtagagacatcatcgttaacgtcatgaagtctttgatgcaattcttggatccagacatctactctgtttgtttcactgattggtctaagttcagattctacgaagctgatttcggttttggtaaaccagtttggactgctgttggtccacaaagaccaattattactaccgccattttgatgaacaacagagaaggtgatggtattgaagcttggttgcatttgaacaagaacgacatgttgatcttcgaacaagacgaagaaatcaagttgttcaccacctga

SEQ ID NO: 13 - DNA, 1278 bp, Artificial Sequence
  Sequence codon optimised for expression in S. cerevisiae
atgaaggtcgaaagattctccagaaagttcattaagccacatactccaactccagaaaacttgaagaagtacaagttgtccttgttggataagtgcttgggtcatgataatttcgccatcgttttgttctacgaatccaagccaagaaacaagtccgaattggaagaatccttggaaaaggttttggttgacttttatccattggctggtagatacaccatgaacgatcatatagttgattgctctgatgttggtgccgtttttgttgaagctgaagctttggatgttgaattgaccatggatgaattggtcaagaacatggaagctcaaaccatccatcatttgttgccaaatcaatacttctctgctgatgctccaaatcctttgttgtctattcaagttacccatttcccatctggtggtttggctattggtattgctgtttctcatgctgttttcgacggtttttctttgggtgttttcgttgctgcttggtctaaagctactatgaatccagatagaaagatcaagatcaccccatcttttgacttgccatctttgttaccatacaaggatgataacttcggtttgactgctgctgaaatcgtttctcaatctgaagatatcgtcgtcaagagattcatcttcggtaaagaagctatcactagattgagatccaagttgtctccaaacagaaacggtaagaagatctccagagttagagttgtttgtgccgttatagttaaggctttgatgggtttggaaagagctaagactagagatttcatgatctgccaaggtatcaacatgagagaaagaacaaaagccccattgcaaaaacatgcttgtggtaatttggccgtttcttcatacactagaagagttgctgctgcagaagcagaagaattgcaatctttggttaacttgatcggtgactccatcgaaaagtctattgctgattacgccgatatcttgtcctctgatcaagatggtagacatatcatctccaccatgatgaagtctttcatgcaatttgctgccccagatattaaggctatttctttcactgattggtctaagttcggtttctaccaagttgattttggtttcggtaaaccagtttggactggtgttagaccagaaagaccaattttttccgctgccattttgatgtctaacagagaaggtgatggtattgaagcttggttgcatttggataagaacgacatgttgatcttcgaacaagacgaagaaatcaagttgttgatcaccacctga

SEQ ID NO: 14 - DNA, 1281 bp, Artificial sequence
  Sequence codon optimised for expression in S. cerevisiae
atgaaggtcgaaagattctccagaaagttcattaagccacatactccaactccagaaaacttgaagaagtacaagttgtccttgttggataagtgcttgggtcatgataatttcgccatcgttttgttctacgaatccaagccaagaaacaagtccgaattggaagaatccttggaaaaggttttggttgacttttatccattggctggtagatacaccatgaacgatcatatagttgattgctctgatgaaggtgccgtttttgttgaagctgaagctccaaatgttgaattgaccgttgatcaattggtcaagaacatggaagctcaaaccatccatgatttcttgccagatcaatactttccagctgatgctcctaatcctttgttgtctattcaagttacccatttcccatgtggtggtttggctattggtatagttgtttctcatgctgttttcgacggtttctctttgggtgtttttttggctgcttggtctaaggctactatgaatccagaaagaaagatcgaaatcaccccatcttttgacttgccatctttgttgccttacaaggacgaatcttttggtttgaacttctccgaaatcgttaaggccgaaaacatcgttgttaagagattgaacttcggtaaagaagccatcaccagattgagatctagattgtctccaaaccataacggtaagaccatctctagagttagagttgtttgtgccttgattgtcaaggctttgatgggtttggaattggctaaacatggtaagactagagacttcatgatctcccaaggtatcaacatgagagaaagaacaaaagccccattgcacaaacatgcttgtggtaatttggccattttgtcttgtaccagaagagttgaagccgaagaaatgatggacttgcaaaacttggttaacttgatcggtgaatccaccgaaaaggatattgctcactactccgaattattgtcccacaatcaattcggtagagacatcatcgttaacgttatgaagtccttgatgcaattcttggatccagatatctactctgtttgtttcactgactggtccaagttcagattttacgaagctgattttggtttcggtaagccagtttggactgctgttggtccacaaagaccaattattactaccgccattttgatgaacaacagagaaggtgatggtattgaagcttggttgcatttgaacaagaacgacatgttgatcttcgaacaagacgaagaaatcaagttgttcaccacctga

SEQ ID NO: 15 - skipped

SEQ ID NO: 16 - protein, 773 aa, Coleus forskohlii
MKMLMIKSQFRVHSIVSAWANNSNKRQSLGHQIRRKQRSQVTECRVASLDALNGIQKVGPATIGTPEEENKKIEDSIEYVKELLKTMGDGRISVSPYDTAIVALIKDLEGGDGPEFPSCLEWIAQNQLADGSWGDHFFCIYDRVVNTAACVVALKSWNVHADKIEKGAVYLKENVHKLKDGKIEHMPAGFEFVVPATLERAKALGIKGLPYDDPFIREIYSAKQTRLTKIPKGMIYESPTSLLYSLDGLEGLEWDKILKLQSADGSFITSVSSTAFVFMHTNDLKCHAFIKNALTNCNGGVPHTYPVDIFARLWAVDRLQRLGISRFFEPEIKYLMDHINNVWREKGVFSSRHSQFADIDDTSMGIRLLKMHGYNVNPNALEHFKQKDGKFTCYADQHIESPSPMYNLYRAAQLRFPGEEILQQALQFAYNFLHENLASNHFQEKWVISDHLIDEVRIGLKMPWYATLPRVEASYYLQHYGGSSDVWIGKTLYRMPEISNDTYKILAQLDFNKCQAQHQLEWMSMKEWYQSNNVKEFGISKKELLLAYFLAAATMFEPERTQERIMWAKTQVVSRMITSFLNKENTMSFDLKIALLTQPQHQINGSEMKNGLAQTLPAAFRQLLKEFDKYTRHQLRNTWNKWLMKLKQGDDNGGADAELLANTLNICAGHNEDILSHYEYTALSSLTNKICQRLSQIQDKKMLEIEEGSIKDKEMELEIQTLVKLVLQETSGGIDRNIKQTFLSVFKTFYYRAYHDAKTIDAHIFQVLFEPVV

SEQ ID NO: 17 - protein, 598 aa, Coleus forskohlii
MSSLAGNLRVIPFSGNRVQTRTGILPVHQTPMITSKSSAAVKCSLTTPTDLMGKIKEVFNREVDTSPAAMTTHSTDIPSNLCIIDTLQRLGIDQYFQSEIDAVLHDTYRLWQLKKKDIFSDITTHAMAFRLLRVKGYEVASDELAPYADQERINLQTIDVPTVVELYRAAQERLTEEDSTLEKLYVWTSAFLKQQLLTDAIPDKKLHKQVEYYLKNYHGILDRMGVRRNLDLYDISHYKSLKAAHRFYNLSNEDILAFARQDFNISQAQHQKELQQLQRWYADCRLDTLKFGRDVVRIGNFLTSAMIGDPELSDLRLAFAKHIVLVTRIDDFFDHGGPKEESYEILELVKEWKEKPAGEYVSEEVEILFTAVYNTVNELAEMAHIEQGRSVKDLLVKLWVEILSVFRIELDTWTNDTALTLEEYLSQSWVSIGCRICILISMQFQGVKLSDEMLQSEECTDLCRYVSMVDRLLNDVQTFEKERKENTGNSVSLLQAAHKDERVINEEEACIKVKELAEYNRRKLMQIVYKTGTIFPRKCKDLFLKACRIGCYLYSSGDEFTSPQQMMEDMKSLVYEPLPISPPEANNASGEKMSCVSN

SEQ ID NO: 18 - protein, 587 aa, Coleus forskohlii
MSITINLRVIAFPGHGVQSRQGIFAVMEFPRNKNTFKSSFAVKCSLSTPTDLMGKIKEKLSEKVDNSVAAMATDSADMPTNLCIVDSLQRLGVEKYFQSEIDTVLDDAYRLWQLKQKDIFSDITTHAMAFRLLRVKGYDVSSEELAPYADQEGMNLQTIDLAAVIELYRAAQERVAEEDSTLEKLYVWTSTFLKQQLLAGAIPDQKLHKQVEYYLKNYHGILDRMGVRKGLDLYDAGYYKALKAADRLVDLCNEDLLAFARQDFNINQAQHRKELEQLQRWYADCRLDKLEFGRDVVRVSNFLTSAILGDPELSEVRLVFAKHIVLVTRIDDFFDHGGPREESHKILELIKEWKEKPAGEYVSKEVEILYTAVYNTVNELAERANVEQGRNVEPFLRTLWVQILSIFKIELDTWSDDTALTLDDYLNNSWVSIGCRICILMSMQFIGMKLPEEMLLSEECVDLCRHVSMVDRLLNDVQTFEKERKENTGNAVSLLLAAHKGERAFSEEEAIAKAKYLADCNRRSLMQIVYKTGTIFPRKCKDMFLKVCRIGCYLYASGDEFTSPQQMMEDMKSLVYEPLQIHPPPAN

SEQ ID NO: 19 - protein, 498 aa, Coleus forskohlii
MELVEVIVVVVGAAALGVVLWSHLKPEGRKLPPGPSPLPIFGNIFQLTGPNTCESFANLSKKYGPVMSLRLGSLFTVVISSPEMAKEVLTNTDFLERPLMQAVHAHDHAQFSIAFLPVTTPKWKQLRRICQEQMFASRILEKSQPLRHQKLQELIDHVQKCCDAGRAVTIRDAAFATTLNLMSVTMFSADATELDSSVTAELRELMAGVVTVLGTPNFADFFPILKYLDPQGVRRKAHFHYGKMFDHIKSRMAERVELKKANPNHLKHDDFLEKILDISLRRDYELTIQDITHLLVDLYVAGSESTVMSIEWIMSELMLHPQSLAKLKAELRSVMGERKMIQESEDISRLPFLNAVIKETLRLHPPGPLLFPRQNTNDVELNGYFIPKGTQILVNEWAIGRDPSVWPNPESFVPERFLDKNIDYKGQDPQLVPFGSGRRICLGIPIAHRMVHSTVAALIHNFEWKFAPDGSEYNRELFSGPALRREVPLNLIPLNPSF

SEQ ID NO: 20 - protein, 492 aa, Coleus forskohlii
METITLLLALFFIALTYFISSRRRRNLPPGPFPLPIIGNMLQLGSKPHQSFAQLSKKYGPLMSIHLGSLYTVIVSSPEMAKEILQKHGQVFSGRTIAQAVHACDHDKISMGFLPVANTWRDMRKICKEQMFSHHSLEASEELRHQKLQQLLDYAQKCCEAGRAVDIREASFITTLNLMSATMFSTQATEFDSEATKEFKEIIEGVATIVGVANFADYFPILKPFDLQGIKRRADGYFGRLLKLIEGYLNERLESRRLNPDAPRKKDFLETLVDIIEANEYKLTTEHLTHLMLDLFVGGSETNTTSLEWIMSELVINPDKMAKVKEELKSVVGDEKLVNESDMPRLPYLQAVIKEVLRIHPPGPLLLPRKAESDQVVNGYLIPKGTQILFNAWAMGRDPTIWKDPESFEPERFLNQSIDFKGQDFELIPFGSGRRICPGMPLANRILHMTTATLVHNFDWKLEEGTADADHKGELFGLAVRRATPLRIIPLKP

SEQ ID NO: 21 - protein, 495 aa, Coleus forskohlii
MELVQVIAVVAVVVVLWSQLKRKGRKLPPGPSPLPIVGNIFQLSGKNINESFAKLSKIYGPVMSLRLGSLLTVIISSPEMAKEVLTSKDFANRPLTEAAHAHGHSKFSVGFVPVSDPKWKQMRRVCQEEMFASRILENSQQRRHQKLQELIDHVQESRDAGRAVTIRDPVFATTLNIMSLTLFSADATEFSSSATAELRDIMAGVVSVLGAANLADFFPILKYFDPQGMRRKADLHYGRLIDHIKSRMDKRSELKKANPNHPKHDDFLEKIIDITIQRNYDLTINEITHLLVDLYLAGSESTVMTIEWTMAELMLRPESLAKLKAELRSVMGERKMIQESDDISRLPYLNGAIKEALRLHPPGPLLFARKSEIDVELSGYFIPKGTQILVNEWGMGRDPSVWPNPECFQPERFLDKNIDYKGQDPQLIPFGAGRRICPGIPIAHRVVHSVVAALVHNFDWEFAPGGSQCNNEFFTGAALVREVPLKLIPLNPPSI

SEQ ID NO: 22 - protein, 493 aa, Coleus forskohlii
METMTLLLPLFFIALTYFLSWRRRRNLPPGPFPLPIIGNLLQIGSKPHQSFAQLSKKYGPLMSVQLGSVYTVIASSPEMAKEILQKHGQVFSGRTIAQAAQACGHDQISIGFLPVATTWRDMRKICKEQMFSHHSLESSKELRHEKLQKLLDYAQKCCEAGRAVDIREAAFITTLNLMSATLFSTQATEFDSEATKEFKEVIEGVAVIVGEPNFADYFPILKPFDLQGIKRRANSYFGRLLKLMERYLNERLESRRLNPDAPKKNDFLETLVDIIQADEYKLTTDHVTHLMLDLFVGGSETSATSLEWIMSELVSNPSKLAKVKAELKSVVGEKKVVSESEMARLPYLQAVIKEVLRLHPPGPLLLPRKAGSDQVVNGYLIPKGTQLLFNVWAMGRDPSIWKNPESFEPERFLNQNIDYKGQDFELIPFGSGRRICPGMPLADRIMHMTTATLVHNFDWKLEDGAGDADHKGDDPFGLAIRRATPLRIIPLKP

SEQ ID NO: 23 - protein, 493 aa, Coleus forskohlii
MESMNALVVGLLLIALTILFSLRRRRNLAPGPYPFPIIGNMLQLGTKPHQSFAQLSKKYGPLMSIHLGSLYTVIVSSPEMAKEILQKHGQVFSGRTIAQAVHACDHDKISMGFLPVSNTWRDMRKICKEQMFSHHSLEGSQGLRQQKLLQLLDYAQKCCETGRAVDIREASFITTLNLMSATMFSTQATEFESKSTQEFKEIIEGVATIVGVANFGDYFPILKPFDLQGIKRKADGYFGRLLKLIEGYLNERLESRKSNPNAPRKNDFLETVVDILEANEYKLSVDHLTHLMLDLFVGGSETNTTSLEWTMSELVNNPDKMAKLKQELKSVVGERKLVDESEMPRLPYLQAVIKESLRIHPPGPLLLPRKAETDQEVNGYLIPKGTQILFNVWAMGRDPSIWKDPESFEPERFLNQNIDFKGQDFELIPFGSGRRICPGMPLANRILHMATATMVHNFDWKLEQGTDEADAKGELFGLAVRRAVPLRIIPLQP

SEQ ID NO: 24 - protein, 424 aa, Artificial sequence
  Chimeric sequence comprising sequences from two ACTs from Coleus forskohlii
MKVERFSRKFIKPHTPTPENLKKYKLSLLDKCLGHDNFAIVLFYESKPRNKSELEESLEKVLVDFYPLAGRHTMNDHIVDCSDVGAVFVEAEALDVELTMDELVKNMEAQTIHHLLPNQYFSADAPNPLLSIQVTHFPSGGLAIGIAVSHAVFDGFSLGVFVAAWSKATMNPDRKIKITPSFDLPSLLPYKDDNFGLTAAEIVSQSEDIVVKRFIFGKEAITRLRSKLSPNRNGKKISRVRVVCAVIVKALMGLERAKTRDFMICQGINMRERTKAPLQKHACGNLAVSSYTRRVAAAEAEELQSLVNLIGDSIEKSIADYADILSSDQDGRHIISTMMKSFMQFAAPDIKAISFTDWSKFGFYQVDFGFGKPVWTGVRPERPIFSAAILMSNREGDGIEAWLHLDKNDMLIFEQDEEIKLFTT

SEQ ID NO: 25 - DNA, 1275 bp, Artificial sequence
  Chimeric sequence comprising sequences from two ACTs from Coleus forskohlii
atgaaggtggaaagatttagcaggaaattcataaaaccacacaccccgacccccgaaaatctgaagaagtataagctttctcttttagacaaatgcttggggcacgataattttgctattgttctgttttacgaatcgaaaccaagaaacaagagtgagttggaagaatcactggaaaaagtcctggtggatttctaccctcttgctggaagacacaccatgaatgatcacatagttgattgcagtgatgtgggcgccgtgtttgtcgaagcagaagctctagacgtcgagctgacgatggatgagctcgttaagaacatggaggcccagactatccatcatctccttcccaatcaatatttctcagctgatgctccaaatccactcttgtcaatccaggtgacacatttcccatctggtggcctagcaattggcatcgccgtttctcacgctgtcttcgatggtttttcgctgggggtgttcgtcgccgcctggtcgaaggccaccatgaacccagatcggaagatcaaaatcacaccgtctttcgatcttccctcgttgcttccttacaaggatgacaattttggattaactgccgccgaaattgtcagccagagtgaagatattgttgttaagaggtttattttcgggaaggaggcaataacgaggttgagatcaaagctaagcccaaatcgcaatgggaaaaagatctctcgtgttcgagtggtgtgtgccgttatagtgaaagccttgatgggcctggaacgcgccaaaacaagagatttcatgatctgtcaagggatcaacatgcgtgagagaacaaaggcacctctacagaagcatgcttgtgggaatctggcggtctcgtcttacacgcgacgtgtagccgcagcagaagccgaggagctgcagagtttggtaaatctaataggtgattctattgagaagagcatagctgattatgctgatattctttcttctgatcaagatgggcgtcatatcatcagcacgatgatgaaatctttcatgcaattcgcggctcctgacataaaagctatatccttcactgattggagtaagtttggattctaccaagttgattttgggttcgggaagcctgtttggacgggtgttcggcccgaacgcccaatctttagcgccgctattctcatgagcaacagagagggtgatggaattgaggcatggcttcatcttgacaaaaatgacatgcttatcttcgagcaagatgaagaaatcaaattatttactacctaa

SEQ ID NO: 26 - protein, 424 aa, Coleus forskohlii
MKVERISRKFIKPYTPTPQNLKKYKLSLLDKCMGHMDFAVVLFYESKPRNKNELEESLEKVLVDFYPLAGRYTMNDHIVDCSDEGAVFVEAEAPNVELTVDQLVKNMEAQTIHDFLPDQYFPADAPNPLLSIQVTHFPCGGLAIGIVVSHAVFDGFSLGVFLAAWSKATMNPERKIEITPSFDLPSLLPYKDESFGLNFSEIVKAENIVVKRLNFGKEAITRLRSKLSPNQNGKTISRVRVVCAVIVKALMGLERAKTRDFMICQGINMRERTKAPLQKHACGNLAVSSYTRRVAAAEAEELQSLVNLIGDSIEKSIADYADILSSDQDGRHIISTMMKSFMQFAAPDIKAISFTDWSKFGFYQVDFGFGKPVWTGVRPERPIFSAAILMSNREGDGIEAWLHLDKNDMLIFEQDEEIKLLITT

SEQ ID NO: 27 - DNA, 1275 bp, Coleus forskohlii
atgaaggttgaaagaataagcaggaaattcataaaaccatacaccccaaccccccaaaacctcaagaaatataagctttctcttttagacaaatgcatggggcacatggattttgctgttgttctgttttatgaatcgaaaccaagaaacaagaatgaattggaagaatcactagaaaaagtcctggtcgatttctaccctcttgctggaagatacaccatgaatgatcacatagttgattgcagtgatgagggcgccgtgtttgtcgaagccgaagctccaaacgtcgagctgacggtggatcaactcgtcaagaacatggaggcccagactatccatgatttccttcccgatcaatatttcccagctgatgctccaaatccactcctctcgatccaggtcacgcatttcccatgtggtggcttggcgattggcatcgttgtttctcacgctgtcttcgatggtttttcgctgggggtgttccttgccgcctggtcgaaggccaccatgaacccagagaggaagatcgaaatcaccccgtctttcgatcttccttcgttgcttccatacaaggatgaaagtttcggattaaatttcagcgaaattgtcaaggctgaaaatattgttgtgaagaggcttaatttcgggaaggaggctataacgaggttgagatcaaaactaagcccaaatcaaaatgggaaaacgatctctcgcgttcgagttgtttgcgccgttatagtgaaagccttgatgggcctggaacgcgccaaaacaagagatttcatgatctgtcaagggatcaacatgcgtgagagaacaaaggcacctctacagaagcatgcttgtgggaatctggcggtctcgtcttacacgcgacgtgtagccgcagcagaagccgaggagctgcagagtttggtaaatctaataggtgattctattgagaagagcatagctgattatgctgatattctttcttctgatcaagatgggcgtcatatcatcagcacgatgatgaaatctttcatgcaattcgcggctcctgacataaaagctatatccttcactgattggagtaagtttggattctaccaagttgattttgggttcgggaagcctgtttggacgggtgttcggcccgaacgcccaatctttagcgccgctattctcatgagcaacagagagggtgatggaattgaggcatggcttcatcttgacaaaaatgacatgcttatcttcgagcaagatgaagaaatcaaattattaattactacctaa

SEQ ID NO: 28 - DNA, 1275 bp, Artificial Sequence
  Sequence codon optimised for expression in S. cerevisiae
atgaaggtcgaaagaatctccagaaagttcattaagccatacactccaactccacaaaacttgaagaagtacaagttgtccttgttggataagtgcatgggtcatatggatttcgctgttgttttgttctacgaatccaagccaagaaacaagaacgaattggaagaatccttggaaaaggttttggttgacttttatccattggctggtagatacaccatgaacgatcatatagttgattgctctgatgaaggtgccgtttttgttgaagctgaagctccaaatgttgaattgaccgttgatcaattggtcaagaacatggaagctcaaaccatccatgatttcttgccagatcaatactttccagctgatgctcctaatcctttgttgtctattcaagttacccatttcccatgtggtggtttggctattggtatagttgtttctcatgctgttttcgacggtttctctttgggtgtttttttggctgcttggtctaaggctactatgaatccagaaagaaagatcgaaatcaccccatcttttgacttgccatctttgttgccttacaaggatgaatctttcggtttgaacttctccgaaatcgttaaggctgaaaacatcgttgtcaagagattgaacttcggtaaagaagccattaccagattgagatctaagttgtccccaaatcaaaacggtaagaccatctctagagttagagttgtttgtgccgttattgtcaaggctttgatgggtttggaaagagctaagactagagatttcatgatctgccaaggtatcaacatgagagaaagaacaaaagccccattgcaaaaacatgcttgtggtaatttggccgtttcttcatacactagaagagttgctgctgctgaagcagaagaattgcaatctttggttaacttgatcggtgactccatcgaaaagtctattgctgattacgccgatatcttgtcctctgatcaagatggtagacatatcatctccaccatgatgaagtctttcatgcaatttgctgccccagatattaaggctatttctttcactgactggtccaagtttggtttctaccaagttgattttggtttcggtaaaccagtttggactggtgttagaccagaaagaccaattttttccgctgccattttgatgtctaacagagaaggtgatggtattgaagcttggttgcatttggataagaacgacatgttgatcttcgaacaagacgaagaaatcaagttgttgatcaccacctga

SEQ ID NO: 29 - DNA, 2169 bp, Coleus forskohlii
atggcgtcttgtggagctatcgggagtagtttcttgccactgctccattccgacgagtcaagcttgttatctcggcccactgctgctcttcacatcaagaagcagaagttttctgtgggagctgctctgtaccaggataacacgaacgatgtcgttccgagtggagagggtctgacgaggcagaaaccaagaactctgagtttcacgggagagaagccttcaactccaattttggataccatcaactatccaatccacatgaagaatctgtccgtggaggaactggagatattggccgatgaactgagggaggagatagtttacacggtgtcgaaaacgggagggcatttgagctcaagcttgggtgtatcagagctcaccgttgcactgcatcatgtattcaacacacccgatgacaaaatcatctgggatgttggacatcaggcgtatccacacaaaatcttgacagggaggaggtccagaatgcacaccatccgacagactttcgggcttgcagggttccccaagagggatgagagcccgcacgacgcgttcggagctggtcacagctccactagtatttcagctggtctagggatggcggtggggagggacttgctacagaagaacaaccacgtgatctcggtgatcggagacggagccatgacagcggggcaggcatacgaggccatgaacaatgcaggatttcttgattccaatctgatcatcgtgttgaacgacaacaaacaagtgtccctgcctacagccaccgtcgacggccctgctcctcccgtcggagccttgagcaaagccctcaccaagctgcaagcaagcaggaagttccggcagctacgagaagcagcaaaaggcatgactaagcagatgggaaaccaagcacacgaaattgcatccaaggtagacacttacgttaaaggaatgatggggaaaccaggcgcctccctcttcgaggagctcgggatttattacatcggccctgtagatggacataacatcgaagatcttgtctatattttcaagaaagttaaggagatgcctgcgcccggccctgttcttattcacatcatcaccgagaagggcaaaggctaccctccagctgaagttgctgctgacaaaatgcatggtgtggtgaagtttgatccaacaacggggaaacagatgaaggtgaaaacgaagactcaatcatacacccaatacttcgcggagtctctggttgcagaagcagagcaggacgagaaagtggtggcgatccacgcggcgatgggaggcggaacggggctgaacatcttccagaaacggtttcccgaccgatgtttcgatgtcgggatagccgagcagcatgcagtcaccttcgccgcgggtcttgcaacggaaggcctcaagcccttctgcacaatctactcttccttcctgcagcgaggttatgatcaggtggtgcacgatgtggatcttcagaaactcccggtgagattcatgatggacagagctggacttgtgggagctgacggcccaacccattgcggcgccttcgacaccacctacatggcctgcctgcccaacatggtcgtcatggctccctccgatgaggctgagctcatgcacatggtcgccactgccgctgtcattgatgatcgccctagctgcgttaggtaccctagaggaaacggtataggggtgcccctccctccaaacaataaaggaattccattagaggttgggaagggaaggattttgaaagagggtaaccgagttgccattctaggcttcggaactatcgtgcaaaactgtctagcagcagcccaacttcttcaagaacacggcatatccgtgagcgtagccgatgcgagattctgcaagcctctggatggagatctgatcaagaatcttgtgaaggagcacgaagttctcatcactgtggaagagggatccattggaggattcagtgcacatgtctctcatttcttgtccctcaatggactcctcgacggcaatcttaagtggaggcctatggtgctcccagataggtacattgatcatggagcataccctgatcagattgaggaagcagggctgagctcaaagcatattgcaggaactgttttgtcacttattggtggagggaaagacagtcttcatttgatcaacatgtaa

SEQ ID NO: 30 - protein, 722 aa, Coleus forskohlii
MASCGAIGSSFLPLLHSDESSLLSRPTAALHIKKQKFSVGAALYQDNTNDVVPSGEGLTRQKPRTLSFTGEKPSTPILDTINYPIHMKNLSVEELEILADELREEIVYTVSKTGGHLSSSLGVSELTVALHHVFNTPDDKIIWDVGHQAYPHKILTGRRSRMHTIRQTFGLAGFPKRDESPHDAFGAGHSSTSISAGLGMAVGRDLLQKNNHVISVIGDGAMTAGQAYEAMNNAGFLDSNLIIVLNDNKQVSLPTATVDGPAPPVGALSKALTKLQASRKFRQLREAAKGMTKQMGNQAHEIASKVDTYVKGMMGKPGASLFEELGIYYIGPVDGHNIEDLVYIFKKVKEMPAPGPVLIHIITEKGKGYPPAEVAADKMHGVVKFDPTTGKQMKVKTKTQSYTQYFAESLVAEAEQDEKVVAIHAAMGGGTGLNIFQKRFPDRCFDVGIAEQHAVTFAAGLATEGLKPFCTIYSSFLQRGYDQVVHDVDLQKLPVRFMMDRAGLVGADGPTHCGAFDTTYMACLPNMVVMAPSDEAELMHMVATAAVIDDRPSCVRYPRGNGIGVPLPPNNKGIPLEVGKGRILKEGNRVAILGFGTIVQNCLAAAQLLQEHGISVSVADARFCKPLDGDLIKNLVKEHEVLITVEEGSIGGFSAHVSHFLSLNGLLDGNLKWRPMVLPDRYIDHGAYPDQIEEAGLSSKHIAGTVLSLIGGGKDSLHLINM

SEQ ID NO: 31 - DNA, 1083 bp, Coleus forskohlii
atgaggtctatgaatctggtcgatgcttgggttcaaaacctccccattttcaagcaaccacacccctccaaattcatccaccatcccagattcgagcccgctttcctcaaatcgcggaggcccatttcctccttcgccgtctccgccgtcctcaccggcgaggaagcaagaatcttcacccgaggagatgaagcgcccttcaatttcaacgcctacgtcgtcgagaaagccacccacgtgaacaaggctctcgacgacgcggtggcggtgaagaaccctccgatgatccacgaggccatgaggtactccttgctcgccggcggaaagagggtccgccccatgctctgcatcgccgcctgcgaggtggtgggcggcccccaagcggcggcgatccccgccgcctgcgcggtggagatgatccacaccatgtctctcatccacgatgatcttccctgtatggacaatgatgacctccgccgcggcaagcccaccaatcacaaagtcttcggcgagaacgtcgccgtgctcgccggtgatgctttattggccttcgcgtttgaattcatcgccactgccaccacgggggtggcccctgagaggattcttgcggcggtggcggagttggcgaaggcgatcgggacggaggggctggtggcggggcaggtggtggatttgcattgcaccggcaatcccaatgtaggactggacacattggaattcatacacatacacaaaactgcagcattgcttgaggcctctgtagttttgggggccattttgggaggaggaagcagtgatcaagttgagaaactgagaacttttgctagaaaaattgggcttctcttccaagtggtggatgacattttagatgtcacaaaatcctcggaggagttggggaagacggccggcaaagacttggccgtcgacaagaccacctacccaaagcttctgggattggagaaagctatggagtttgctgagaggctgaatgaggaggccaagcagcagctgctggattttgacccccggaaggcggcgccgctggtggcgctggccgattacattgctcacaggcagaactag

SEQ ID NO: 32 - protein, 360 aa, Coleus forskohlii
MRSMNLVDAWVQNLPIFKQPHPSKFIHHPRFEPAFLKSRRPISSFAVSAVLTGEEARIFTRGDEAPFNFNAYVVEKATHVNKALDDAVAVKNPPMIHEAMRYSLLAGGKRVRPMLCIAACEVVGGPQAAAIPAACAVEMIHTMSLIHDDLPCMDNDDLRRGKPTNHKVFGENVAVLAGDALLAFAFEFIATATTGVAPERILAAVAELAKAIGTEGLVAGQVVDLHCTGNPNVGLDTLEFIHIHKTAALLEASVVLGAILGGGSSDQVEKLRTFARKIGLLFQVVDDILDVTKSSEELGKTAGKDLAVDKTTYPKLLGLEKAMEFAERLNEEAKQQLLDFDPRKAAPLVALADYIAHRQN

SEQ ID NO: 33 - DNA, 2136 bp, Coleus forskohlii
atggaatcgactattgagaagctttcgcccttcgatttgatgactgcgattctcaaaggagtcaaacttgataattcgaacgggtctgctggggtggagcatccggctgtgatcgcgatgctgatggagaacaaggatctcgtgatgatgctcaccacctccgtcgcggtgcttctaggacttgctgtgtatctcgtgtggcggcgcggagccggatcggcgaagagggtggtggagccgccgaagctggtgattcccaagggcccggtggatgcggaggaagaggatgatgggaagaagaaggttaccatcttttttgggacgcagactggaactgctgaaggctttgctaaggcacttgccgaagaagctaaagcaagatatccgctgaccaactttaaagtagttgacttggatgattatgctgccgatgatgaagagtatgaagagaagatgaagaaggagacctttgcattcttcttcttggcgacatatggagatggtgagcctaccgacaatgctgcgagattttacaagtggttttccgaggggaaagagagaggtgagatattcaagaatctcaactatggtgtatttggtcttggaaacaggcagtatgagcatttcaacaagattgctatagtggtggatgacattcttcttgagcaaggtggaaatcggcttgtccctgtgggtcttggagatgacgatcaatgtatcgaagatgatttctcagcatggcgtgataatgtgtggcctgagctggataagttgctccgtgatgaggatgatgcaactgttgcaactccatatactgcagccgttttggagtatcgtgttgtgttccatgaccagtcagatgaactgcactcggaaaacaacttagccaatggtcatgcaaatggaaatgcttcttatgatgctcaacacccctgcaaagtgaatgttgctgtaaaaagggagctacatactcctctatccgatcgttcttgcactcacttggaattcgacatatctggcactggattagagtatgaaacaggggaccacgttggtgtttactgtgagaacttgattgaaactgtagaggaagcagaaaggcttcttggtctttctccacaaacattcttttcagttcacactgataaagcggacggcacaccacttggtggaagtgccttgcctcctcccttcccgccgtgcactttgaggacagcgctaagtcgatatgctgatcttttgaatgctcccaaaaagtctgctttgactgcattggctgcttatgcctctgaccctagtgaagctgatcggctcaagcaccttgcttcccctgatggaaaggaggaatatgctcaatatgtggtttctggtcagagaagcctacttgaggtgatggctgacttcccatctgccaagcctcctcttggtgttttctttgctgcaattgctcctcgcttgcagcctcgattttattcaatctcatcctcaccaaagattgcaccttcaagaattcacgtcacttgtgcgttggtgtatgagaaaatgcccactggacgaatccacaagggtgtctgctcaacatggatgaagaatgctgtgccattggaggaaagccccaactgctcttcagcaccagtttttgtacggacctcaaacttcagactccctgctgatcctaaagtaccagttataatgattggccctggaaccggtttggctccattcaggggttttcttcaggaaagattagccctcaaggaatctggagcagaacttggtcctgctatattattcttcgggtgcagaaacagtaaaatggatttcatttaccaagatgaactggataactttgttaaagctggagtggtttctgagcttgtccttgcgttttcacgcgagggtcctgctaaggaatacgtgcagcataagatggcacagaaggcctcggatgtgtggaatatgatatcagaagggggctacgtttatgtatgtggtgatgctaagggcatggcacgtgacgttcaccggactcttcacaccattgttcaagaacagggatctctggacagctcgaaaaccgagagcttcgtcaagaatctgcagatgaccggccggtacctgcgtgacgtgtggtga

SEQ ID NO: 34 - protein, 711 aa, Coleus forskohlii
MESTIEKLSPFDLMTAILKGVKLDNSNGSAGVEHPAVIAMLMENKDLVMMLTTSVAVLLGLAVYLVWRRGAGSAKRVVEPPKLVIPKGPVDAEEEDDGKKKVTIFFGTQTGTAEGFAKALAEEAKARYPLTNFKVVDLDDYAADDEEYEEKMKKETFAFFFLATYGDGEPTDNAARFYKWFSEGKERGEIFKNLNYGVFGLGNRQYEHFNKIAIVVDDILLEQGGNRLVPVGLGDDDQCIEDDFSAWRDNVWPELDKLLRDEDDATVATPYTAAVLEYRVVFHDQSDELHSENNLANGHANGNASYDAQHPCKVNVAVKRELHTPLSDRSCTHLEFDISGTGLEYETGDHVGVYCENLIETVEEAERLLGLSPQTFFSVHTDKADGTPLGGSALPPPFPPCTLRTALSRYADLLNAPKKSALTALAAYASDPSEADRLKHLASPDGKEEYAQYVVSGQRSLLEVMADFPSAKPPLGVFFAAIAPRLQPRFYSISSSPKIAPSRIHVTCALVYEKMPTGRIHKGVCSTWMKNAVPLEESPNCSSAPVFVRTSNFRLPADPKVPVIMIGPGTGLAPFRGFLQERLALKESGAELGPAILFFGCRNSKMDFIYQDELDNFVKAGVVSELVLAFSREGPAKEYVQHKMAQKASDVWNMISEGGYVYVCGDAKGMARDVHRTLHTIVQEQGSLDSSKTESFVKNLQMTGRYLRDVW

SEQ ID NO: 35 - DNA, 2196 bp, Artificial Sequence
  Sequence codon optimised for expression in S. cerevisiae
atgtccagagttgcttccttggatgctttgaatggtattcaaaaagttggtccagctaccattggtactccagaagaagaaaacaagaagatcgaagattccatcgaatacgtcaaagaattattgaaaaccatgggtgacggtagaatctctgtttctccatatgatactgctatcgtcgccttgattaaggatttggaaggtggtgatggtccagaatttccatcttgtttggaatggattgcccaaaatcaattggctgatggttcttggggtgatcattttttctgtatctacgatagagttgttaacaccgctgcttgtgttgttgctttgaaatcttggaatgttcacgccgataagattgaaaaaggtgccgtttacttgaaagaaaacgtccacaaattgaaggacggtaagatagaacatatgccagctggttttgaattcgttgttccagcaactttggaaagagctaaagctttgggtattaagggtttgccatatgatgatccattcatcagagaaatctactccgctaagcaaactagattgactaagattccaaagggtatgatctacgaatctccaacctctttgttgtactctttggatggtttagaaggtttggaatgggataagatcttgaagttgcaatcagctgacggttctttcatcacttctgtttcttctactgccttcgttttcatgcataccaacgatttgaagtgccatgcctttattaagaacgctttgactaactgtaatggtggtgttccacatacttacccagttgatatttttgctagattgtgggccgttgacagattgcaaagattgggtatttctagattcttcgaaccagaaatcaaatacttgatggaccacatcaacaacgtttggagagaaaagggtgttttctcatccagacattctcaattcgccgatattgatgatacctccatgggtatcagattattgaagatgcatggttacaacgttaacccaaacgctttggaacatttcaagcaaaaggatggtaaattcacctgttacgccgatcaacatattgaatctccatctccaatgtataacttgtacagagctgcccaattgagatttccaggtgaagaaattttacaacaagccttgcaattcgcctacaacttcttgcacgaaaatttggcttctaaccacttccaagaaaagtgggttatctccgatcatttgatcgatgaagttagaatcggtttgaaaatgccatggtatgctactttgccaagagttgaagcttcttactacttgcaacattacggtggttcttccgatgtttggattggtaaaaccttgtatagaatgccagaaatctctaacgacacctacaagattttggctcaattggatttcaacaagtgccaagctcaacatcaattagaatggatgtctatgaaggaatggtatcaatccaacaacgtaaaagaattcggtatctccaagaaagaattgttgttggcttactttttggctgctgctactatgtttgaacctgaaagaactcaagaaagaatcatgtgggctaagacccaagttgtttctagaatgattacctcattcttgaacaaagaaaacactatgtccttcgacttgaagattgctttgttgactcaaccacaacaccaaatcaatggttccgaaatgaagaatggtttggcacaaactttaccagctgccttcagacaattattgaaagaattcgacaagtacaccagacaccaattgagaaatacttggaacaagtggttgatgaagttgaagcaaggtgatgataacggtggtgctgatgctgaattattggctaacactttgaacatttgcgccggtcataacgaagatattttgtcccattacgaatacaccgccttgtcatctttgaccaacaagatttgtcaaagattgtcccaaatccaagataagaagatgttggaaatcgaagaaggttccatcaaggacaaagaaatggaattggaaattcaaaccttggtcaagttggtattgcaagaaacttctggtggtatcgacagaaacatcaagcaaactttcttgtccgttttcaagaccttctactacagagcttaccatgatgctaagaccattgatgcccatatcttccaagttttgttcgaacctgttgtttaa

SEQ ID NO: 36 - DNA, 1704 bp, Artificial Sequence
  Sequence codon optimised for expression in S. cerevisiae
atgatcacctccaaatcttccgctgctgttaagtgttctttgactactccaactgatttgatgggtaagatcaaagaagttttcaacagagaagttgatacctctccagctgctatgactactcattctactgatattccatccaacttgtgcatcatcgataccttgcaaagattgggtatcgaccaatacttccaatccgaaattgatgctgtcttgcatgatacttacagattgtggcaattgaagaagaaggacatcttctctgatattaccactcatgctatggccttcagattattgagagttaagggttacgaagttgcctctgatgaattggctccatatgctgatcaagaaagaatcaacttgcaaaccattgatgttccaaccgtcgtcgaattatacagagctgcacaagaaagattgaccgaagaagattctaccttggaaaagttgtacgtttggacttctgctttcttgaagcaacaattattgaccgatgccatcccagataagaagttgcataagcaagtcgaatattacttgaagaactaccacggtatcttggatagaatgggtgttagaagaaacttggacttgtacgatatctcccactacaaatctttgaaggctgctcatagattctacaacttgtctaacgaagatattttggccttcgccagacaagatttcaacatttctcaagcccaacaccaaaaagaattgcaacaattgcaaagatggtacgccgattgcagattggatactttgaaattcggtagagatgtcgtcagaatcggtaactttttaacctctgctatgatcggtgatccagaattgtctgatttgagattggcttttgctaagcacatcgttttggttaccagaatcgatgatttcttcgatcatggtggtccaaaagaagaatcctacgaaattttggaattggtcaaagaatggaaagaaaagccagctggtgaatacgtttctgaagaagtcgaaatcttattcaccgctgtttacaacaccgttaacgaattggctgaaatggcccatattgaacaaggtagatctgttaaggatttgttggttaagttgtgggtcgaaatattgtccgttttcagaatcgaattggatacctggactaacgatactgctttgactttggaagaatacttgtcccaatcctgggtttctattggttgcagaatctgcattttgatctccatgcaattccaaggtgttaagttgagtgacgaaatgttgcaaagtgaagaatgtaccgatttgtgcagatacgtttccatggtcgatagattattgaacgatgtccaaaccttcgaaaaagaaagaaaagaaaacaccggtaactccgtttctttgttgcaagctgctcacaaagacgaaagagttatcaacgaagaagaagcctgcatcaaggtaaaagaattagccgaatacaatagaagaaagttgatgcaaatcgtctacaagaccggtactattttcccaagaaaatgcaaggacttgttcttgaaggcttgtagaattggttgctacttgtactcttctggtgatgaattcacttccccacaacaaatgatggaagatatgaagtccttggtctatgaaccattgccaatttctccacctgaagctaacaatgcatctggtgaaaaaatgtcctgcgtcagtaactga

SEQ ID NO: 37 - protein, 297 aa, Synechococcus sp.
MVAQTFNLDTYLSQRQQQVEEALSAALVPAYPERIYEAMRYSLLAGGKRLRPILCLAACELAGGSVEQAMPTACALEMIHTMSLIHDDLPAMDNDDFRRGKPTNHKVFGEDIAILAGDALLAYAFEHIASQTRGVPPQLVLQVIARIGHAVAATGLVGGQVVDLESEGKAISLETLEYIHSHKTGALLEASVVSGGILAGADEELLARLSHYARDIGLAFQIVDDILDVTATSEQLGKTAGKDQAAAKATYPSLLGLEASRQKAEELIQSAKEALRPYGSQAEPLLALADFITRRQH

SEQ ID NO: 38 - DNA, 894 bp, Artificial Sequence
  Sequence codon optimised for expression in S. cerevisiae
atggtcgcacaaactttcaacctggatacctacttatcccaaagacaacaacaagttgaagaggccctaagtgctgctcttgtgccagcttatcctgagagaatatacgaagctatgagatactccctcctggcaggtggcaaaagattaagacctatcttatgtttagctgcttgcgaattggcaggtggttctgttgaacaagccatgccaactgcgtgtgcacttgaaatgatccatacaatgtcactaattcatgatgacctgccagccatggataacgatgatttcagaagaggaaagccaactaatcacaaggtgttcggggaagatatagccatcttagcgggtgatgcgcttttagcttacgcttttgaacatattgcttctcaaacaagaggagtaccacctcaattggtgctacaagttattgctagaatcggacacgccgttgctgcaacaggcctcgttggaggccaagtcgtagaccttgaatctgaaggtaaagctatttccttagaaacattggagtatattcactcacataagactggagccttgctggaagcatcagttgtctcaggcggtattctcgcaggggcagatgaagagcttttggccagattgtctcattacgctagagatataggcttggcttttcaaatcgtcgatgatatcctggatgttactgctacatctgaacagttggggaaaaccgctggtaaagaccaggcagccgcaaaggcaacttatccaagtctattgggtttagaagcctctagacagaaagcggaagagttgattcaatctgctaaggaagccttaagaccttacggttcacaagcagagccactcctagcgctggcagacttcatcacacgtcgtcagcattaa

(19) United States
(12) Patent Application Publication
(10) Pub. No.: US 2018/0112243 A1
(43) Pub. Date: Apr. 26, 2018

(54) BIOSYNTHESIS OF ACETYLATED 13R-MO AND RELATED COMPOUNDS

(71) Applicants: EVOLVA SA, Reinach (CH); University of Copenhagen, Copenhagen (DK)

(72) Inventors: Björn Hamberger, Okemos, MI (US); Johan Andersen-Ranberg, Berkeley, CA (US); Niels Bjerg Jensen, Kastrup (DK); Eirini Pateraki, København NV (DK); Birger Lindberg Møller, Brønshøj (DK)

(21) Appl. No.: 15/565,956

(22) PCT Filed: Apr. 14, 2016
(86) PCT No.: PCT/EP2016/058270

§ 371 Date: Oct. 12, 2017

(30) Foreign Application Priority Data
Apr. 15, 2015 (DK) PA201570220
May 29, 2015 (DK) PA201570330

(51) Int. Cl. C12P 17/06; C12N 9/10

(52) U.S. Cl. CPC C12P 17/06; C12N 9/1029

(57) ABSTRACT

The invention relates to recombinant microorganisms and methods for producing acetylated diterpenes, including oxidized and/or acetylated oxidized diterpenes such as forskolin.

### BACKGROUND OF THE INVENTION

### Field of the Invention

The present invention relates to the field of biosynthesis of substituted diterpenes. More specifically, the invention relates to methods for biosynthesis of acetylated diterpenes, such as methods for biosynthesis of acetylated 13R-manoyl oxide (13R-MO), acetylated oxidized 13R-MO, and related compounds, including biosynthesis of forskolin.

### Description of Related Art

Forskolin is a complex functionalized derivative of 13R-MO requiring regio- and stereospecific oxidation of five carbon positions. Forskolin is a diterpene naturally produced by Coleus forskohlii. Forskolin, oxidized variants of forskolin, and/or acetylated variants of forskolin have been suggested as useful in treatment of a number of clinical conditions. Forskolin has been shown to decrease intraocular pressure and can be used as an antiglaucoma agent in the form of eye drops. See Wagh et al., 2012, J Postgrad Med. 58(3)199-202. Moreover, a water-soluble analogue of forskolin (NKH477), which has been shown to have vasodilatory effects when administered intravenously, has been approved for commercial use in Japan for treatment of acute heart failure and heart surgery complications. See Kikura et al., 2004, Pharmacol Res 49:275-81. Forskolin, which also acts as bronchodilator, can be used for asthma treatments. See Yousif & Thulesius, 1999, J Pharm Pharmacol. 51(2):181-6. In addition, forskolin may help to treat obesity by contributing to higher rates of body fat burning and promoting lean body mass formation. See Godard et al., 2005, Obes Res. 13:1335-43.

Forskolin has been previously purified from C. forskohlii roots using non-environmental friendly organic solvents or produced chemically by cost ineffective procedures (Delpech et al., 1996, Tetrahedron Letters 37(7): 1019-22. Acetylated 13R-MO and acetylated oxidized 13R-MO can be valuable on its own account or as precursors for production of forskolin. See Matsingou & Demetzos, 2007, J Liposome Res. 17(2):89-105 and Fokialakis et al., 2006, Biol Pharm Bull. 29(8):1775-8. Therefore, there remains a need in the art for methods for biosynthesis of forskolin and other acetylated diterpenes.

### SUMMARY OF THE INVENTION

It is against the above background that the present invention provides certain advantages and advancements over the prior art.

Although this invention as disclosed herein is not limited to specific advantages or functionalities, the invention provides a method of producing an acetylated diterpene, comprising:
(a) providing a recombinant host cell capable of producing a diterpene, wherein the recombinant host cell comprises a gene encoding a diterpene acetyltransferase polypeptide capable of catalyzing acetylation of the diterpene;
   wherein the gene is a recombinant gene; and
(b) incubating the recombinant host cell under conditions in which the gene is expressed;

wherein the acetylated diterpene is produced by the recombinant host cell.

In one aspect of the method disclosed herein, the diterpene is 13R-manoyl oxide (13R-MO) or a 13R-MO derivative.

In one aspect of the method disclosed herein, the 13R-MO derivative is an oxidized 13R-MO derivative.

In one aspect of the method disclosed herein, the diterpene acetyltransferase polypeptide is a polypeptide having at least 55% identity to an amino acid sequence set forth in SEQ ID NO:6, SEQ ID NO:7, SEQ ID NO:8, SEQ ID NO:9, SEQ ID NO:10, SEQ ID NO:24, and/or SEQ ID NO:26.

In one aspect of the method disclosed herein, the acetylated diterpene is the acetylated diterpene of formula (I)

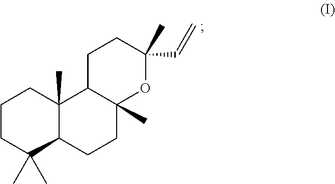 [SMILES: C=C[C@@]1(C)CCC2[C@@](C)(CC[C@H]3C(C)(C)CCC[C@]23C)O1]

where at least one hydrogen is replaced with an acetyl group; and

wherein the chemical valence requirement of the acetylated diterpene is satisfied.

In one aspect of the method disclosed herein, the acetylated diterpene is the acetylated diterpene of formula (I) substituted at one or more of the positions 1, 6, 7, 9 and/or 11 with an acetyl group.

In one aspect of the method disclosed herein, the acetylated diterpene is the acetylated diterpene of formula (I)

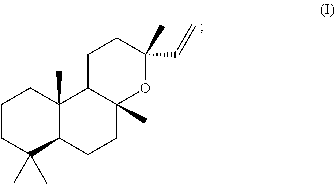 [SMILES: C=C[C@@]1(C)CCC2[C@@](C)(CC[C@H]3C(C)(C)CCC[C@]23C)O1]

where at least one hydrogen is replaced with an acetyl group;

wherein at least one of the other hydrogens is substituted with an —OH and/or ═O group; and

wherein the chemical valence requirement of the acetylated diterpene is satisfied.

In one aspect of the method disclosed herein, the acetylated diterpene is the acetylated diterpene of formula (I) substituted at two or more of the positions 1, 6, 7, 9, and/or 11;

wherein at least one position is substituted with an acetyl group; and

wherein at least one position is substituted with an —OH or =O group.

In one aspect of the method disclosed herein, the recombinant host cell is grown at a temperature for a period of time, wherein the temperature and period of time facilitate the production of the acetylated diterpene.

In one aspect of the method disclosed herein, the recombinant host cell is grown in a fermentor.

In one aspect, the method disclosed herein further comprises isolating the acetylated diterpene.

In one aspect of the method disclosed herein, the acetylated diterpene is forskolin.

The invention further provides a recombinant host cell capable of producing an acetylated diterpene, wherein the recombinant host cell comprises a recombinant gene encoding a diterpene acetyltransferase polypeptide capable of catalyzing acetylation of the diterpene.

In one aspect of the recombinant host cell disclosed herein, the diterpene is 13R-manoyl oxide (13R-MO) or a 13R-MO derivative.

In one aspect of the recombinant host cell disclosed herein, the 13R-M0 derivative is an oxidized 13R-MO derivative.

In one aspect of the recombinant host cell disclosed herein, the diterpene acetyltransferase polypeptide is a polypeptide having at least 55% identity to an amino acid sequence set forth in SEQ ID NO:6, SEQ ID NO:7, SEQ ID NO:8, SEQ ID NO:9, SEQ ID NO:10, SEQ ID NO:24, and/or SEQ ID NO:26.

In one aspect, the recombinant host cell disclosed herein further comprises:
(a) a gene encoding a diterpene synthase polypeptide of class I; and/or
(b) a gene encoding a diterpene synthase polypeptide of class II;
wherein at least one of these genes is a recombinant gene.

In one aspect, the recombinant host cell disclosed herein further comprises:
(a) a gene encoding a TPS2 polypeptide having at least 50% identity to an amino acid sequence set forth in SEQ ID NO:16;
(b) a gene encoding a TPS3 polypeptide having at least 50% identity to an amino acid sequence set forth in SEQ ID NO:17; and/or
(c) a gene encoding a TPS4 polypeptide having at least 40% identity to an amino acid sequence set forth in SEQ ID NO:18;
wherein at least one of these genes is a recombinant gene.

In one aspect, the recombinant host cell disclosed herein further comprises a recombinant gene encoding a polypeptide capable of catalyzing oxidation of 13R-MO.

In one aspect of the recombinant host cell disclosed herein, the gene encoding a polypeptide capable of catalyzing oxidation of 13R-MO comprises:
(a) a CYP76AH16 polypeptide having at least 55% identity to an amino acid sequence set forth in SEQ ID NO:19;
(b) a CYP76AH8 polypeptide having at least 50% identity to an amino acid sequence set forth in SEQ ID NO:20;
(c) a CYP76AH11 polypeptide having at least 50% identity to an amino acid sequence set forth in SEQ ID NO:21;
(d) a CYP76AH15 polypeptide having at least 50% identity to an amino acid sequence set forth in SEQ ID NO:22; and/or
(e) a CYP76AH17 polypeptide having at least 50% identity to an amino acid sequence set forth in SEQ ID NO:23.

In one aspect of the method or the recombinant host cell disclosed herein, the diterpene acetyltransferase polypeptide is a chimeric protein of one or more acetyltransferase polypeptides.

In one aspect of the method or the recombinant host cell disclosed herein, the diterpene acetyltransferase polypeptide is ACT1-3A having an amino acid sequence set forth in SEQ ID NO:8, ACT1-3B having an amino acid sequence set forth in SEQ ID NO:24, and/or ACT1-4 having an amino acid sequence set forth in SEQ ID NO:9.

In one aspect of the recombinant host cell disclosed herein, the recombinant host cell comprises a plant cell, a mammalian cell, an insect cell, a fungal cell, an algal cell, or a bacterial cell.

In one aspect of the recombinant host cell disclosed herein, the bacterial cell comprises Escherichia cells, Lactobacillus cells, Lactococcus cells, Cornebacterium cells, Acetobacter cells, Acinetobacter cells, or Pseudomonas cells.

In one aspect of the recombinant host cell disclosed herein, the fungal cell comprises a yeast cell.

In one aspect of the recombinant host cell disclosed herein, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

In one aspect of the recombinant host cell disclosed herein, the yeast cell is a Saccharomycete.

In one aspect of the recombinant host cell disclosed herein, the yeast cell is a Saccharomyces cerevisiae cell.

In one aspect of the recombinant host cell disclosed herein, the plant cell is a Nicotiana benthamiana cell.

In one aspect of the method disclosed herein, the recombinant host cell is the recombinant host cell disclosed herein.

The invention further provides an acetylated diterpene composition produced by the method disclosed herein.

The invention further provides an acetylated diterpene composition produced by the recombinant host cell disclosed herein.

In one aspect of the acetylated diterpene composition disclosed herein, the acetylated diterpene composition is an acetylated 13R-MO composition.

In one aspect of the acetylated diterpene composition disclosed herein, the acetylated diterpene composition is a forskolin composition.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows the structure of 13R-MO ((3R,4aR,10aS)-3,4a,7,7,10a-pentamethyl-3-vinyldodecahydro-1H-benzo[f]chromene) and formulas for 13R-MO derivatives.

FIG. 2A shows a hypothetical biosynthetic route to forskolin in C. forskohlii proposed by Asada et al., Phytochemistry 79 (2012) 141-146. FIG. 2B shows a reaction capable of being catalyzed by a terpene synthase, such as terpene synthase 2 (TPS2). For example, conversion of geranylgeranyl diphosphate to (5S,8R,9R,10R)-labda-8-ol diphosphate is capable of being catalyzed by TSP2 of SEQ ID NO:16 (see Examples 1-4). FIG. 2C shows a reaction capable of being catalyzed by a terpene synthase, such as TPS3 (SEQ ID NO:17) or TPS4 (SEQ ID NO:18). For example, conversion of (5S,8R,9R,10R)-labda-8-ol diphosphate to 13R-MO is capable of being catalyzed by TPS3 (SEQ ID NO:17) or TPS4 (SEQ ID NO:18).

FIG. 3 shows 13R-MO-derived oxygenated products produced by cytochrome P450 (GYP) 76AH8 (CYP76AH8 of SEQ ID NO:20), CYPAH17 (SEQ ID NO:23), CYPAH15 (SEQ ID NO:22), CYP76AH11 (SEQ ID NO:21), and CYP76AH16 (SEQ ID NO:19). The empirical formulas of the oxygenated products formed by the CYPs are shown. Each compound is marked by a letter and number; compounds identified by the same number are isomers of one another. The structures of 11-oxo-13R-manoyl oxide (compound 2), 9-hydroxy-13R-manoyl oxide (compound 3a), 1,11-dihydroxy-13R-manoyl oxide (compound 5d), 1,9-dideoxydeacetyl-forskolin (compound 7h), and 9-deoxydeacetyl-forskolin (compound 10b) are also shown in FIG. 3.

FIG. 4 shows a liquid chromatography-mass spectrometry (LC-MS) chromatogram (m/z 433) of forskolin-producing S. cerevisiae extracts comprising TPS2 (SEQ ID NO:35, SEQ ID NO:16), TPS3 (SEQ ID NO:36, SEQ ID NO:17), CYP76AH8 (SEQ ID NO:20), CYP76AH11 (SEQ ID NO:21), CYP76AH16 (SEQ ID NO:19), and acetyltransferase (ACT) 1-6 (ACT1-6 of SEQ ID NO:6). An LC-MS spectrum with retention time in min on the x-axis is shown for the extract (upper panel) and for a forskolin standard (lower panel); the forskolin peak is indicated. See Example 1.

FIG. 5 shows production of acetylated 13R-MO in an S. cerevisiae extract comprising CYP76AH16 (SEQ ID NO:19), CYP76AH8 (SEQ ID NO:20), CYP76AH11 (SEQ ID NO:21), and ACT1-6 (SEQ ID NO:6) (dotted lines), as compared to yeast cells comprising CYP76AH16 (SEQ ID NO:19), CYP76AH8 (SEQ ID NO:20), and CYP76AH11 (SEQ ID NO:21) (solid line). The peaks labeled (A) represent oxidized products, i.e. products having one or more —OH or ═O groups, and the peaks labeled (B) represent acetylated products, i.e., products having one or more acetyl groups. See Example 1.

FIG. 6A shows overlaid extraction ion chromatograms (EIC) of (A) a yeast strain comprising CYPAH8 (SEQ ID NO:20), CYPAH11 (SEQ ID NO:21), and CYPAH16 (SEQ ID NO:19) (solid gray line), (B) a yeast strain comprising CYPAH8 (SEQ ID NO:20), CYPAH11 (SEQ ID NO:21), CYPAH16 (SEQ ID NO:19), and ACT1-8 (SEQ ID NO:28, SEQ ID NO:26) (solid black line), and (C) a forskolin standard (dotted line). See Example 2.

FIG. 6B shows overlaid total ion chromatograms (TIC) of (A) a yeast strain comprising CYPAH8 (SEQ ID NO:20), CYPAH11 (SEQ ID NO:21), CYPAH16 (SEQ ID NO:19), and ACT1-8 (SEQ ID NO:28, SEQ ID NO:26) (solid gray line), (B) a forskolin standard (dotted line), and (C) EIC m/z 433 trace (solid black line). See Example 2.

FIG. 7A shows LC-MS chromatograms of extracts of leaves of Nicotiana benthamiana comprising TPS2 (SEQ ID NO:16), TPS3 (SEQ ID NO:17), CYP76AH15 (SEQ ID NO:22), CYP76AH11 (SEQ ID NO:21), and CYP76AH16 (SEQ ID NO:19) in addition to one of ACT1-7 (SEQ ID NO:2, SEQ ID NO:7), ACT1-1 (SEQ ID NO:5, SEQ ID NO:10), ACT1-3B (SEQ ID NO:25, SEQ ID NO:24), ACT1-3A (SEQ ID NO:3, SEQ ID NO:8), or ACT1-6 (SEQ ID NO:1, SEQ ID NO:6), as indicated. An LC-MS spectrum with retention time in min on the x-axis is shown for the extract (lower panels) and for a forskolin standard (top panel). See Example 3.

FIG. 7B shows biosynthesis of forskolin by transient expression of C. forskohlii genes in N. benthamiana as monitored by LC-MS based EIC. Deacetylforskolin accumulation upon expression of TPS2 (SEQ ID NO:16), TPS3 (SEQ ID NO:17), CYP76AH15 (SEQ ID NO:22), CYPAH11 (SEQ ID NO:21), and CYPAH16 (SEQ ID NO:19) is shown in panel 2. Deacetylforskolin and forskolin accumulation upon expression of TPS2 (SEQ ID NO:16), TPS3 (SEQ ID NO:17), CYP76AH15 (SEQ ID NO:22), CYPAH11 (SEQ ID NO:21), CYPAH16 (SEQ ID NO:19), and ACT1-6 (SEQ 1D NO:1, SEQ ID NO:6) is shown in panel 4. Deacetylforskolin and forskolin accumulation upon expression of TPS2 (SEQ ID NO:16), TPS3 (SEQ ID NO:17), CYP76AH15 (SEQ ID NO:22), CYPAH11 (SEQ ID NO:21), CYPAH16 (SEQ ID NO:19), and ACT1-8 (SEQ ID NO:27, SEQ ID NO:26) is shown in panel 5. Deacetylforskolin (13b) and forskolin (16) standards are shown in panels 3 and 6, respectively. See Example 3.

FIG. 7C shows LC-qTOF-MS analysis of 13R-MO derived diterpenoids obtained by transient expression of combinations of C. forskohlii CYP and ACT encoding genes in N. benthamiana. TIC chromatograms from extracts comprising CYP76AH8 (SEQ ID NO:20), CYP76AH11 (SEQ ID NO:21), CYP76AH16 (SEQ ID NO:19), and ACT1-6 (SEQ ID NO:1, SEQ ID NO:6) (top panel) or CYP76AH8 (SEQ ID NO:20), CYP76AH11 (SEQ ID NO:21), CYP76AH16 (SEQ ID NO:19), and ACT1-8 (SEQ ID NO:27, SEQ ID NO:26) (panel 2) are shown. Oxidized and acetylated 13R-MO derived diterpenoids (marked with gray bars). Deacetylforskolin (13b) and forskolin (16c) were confirmed by comparison to authentic standards. See Example 3.

FIG. 8 shows forskolin accumulation (in mg/L) by an S. cerevisiae strain comprising CYP76AH15 (SEQ ID NO:22), CYP76AH11 (SEQ ID NO:21), CYP76AH16 (SEQ ID NO:19), and ACT1-8 (SEQ ID NO:28, SEQ ID NO:26). See Example 4.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE INVENTION

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

l It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker.

As used herein, the terms “microorganism,” “microorganism host,” “microorganism host cell,” “recombinant host,” and “recombinant host cell” can be used interchangeably. As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into a host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In some aspects, said recombinant genes are encoded by cDNA. In other embodiments, recombinant genes are synthetic and/or codon-optimized for expression in S. cerevisiae.

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a yeast gene. In some embodiments, the gene is endogenous to S. cerevisiae, including, but not limited to S. cerevisiae strain S288C. In some embodiments, an endogenous yeast gene is overexpressed. As used herein, the term “overexpress” is used to refer to the expression of a gene in an organism at levels higher than the level of gene expression in a wild type organism. See, e.g., Prelich, 2012, Genetics 190:841-54. See, e.g., Giaever & Nislow, 2014, Genetics 197(2):451-65. As used herein, the terms “deletion,” “deleted,” “knockout,” and “knocked out” can be used interchangeably to refer to an endogenous gene that has been manipulated to no longer be expressed in an organism, including, but not limited to, S. cerevisiae.

As used herein, the terms “heterologous sequence,” “heterologous coding sequence,” and “heterologous gene” are used to describe a sequence derived from a species other than the recombinant host. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. A heterologous nucleic acid may be introduced into a host organism by recombinant methods. Thus, the genome of the host organism can be augmented by at least one incorporated heterologous nucleic acid sequence. It will be appreciated that typically the genome of a recombinant host described herein is augmented through the stable introduction of one or more heterologous nucleic acids encoding one or more enzymes. In some embodiments, a coding sequence is a sequence that is native to the host.

A “selectable marker” can be one of any number of genes that complement host cell auxotrophy, provide antibiotic resistance, or result in a color change. Linearized DNA fragments of the gene replacement vector then are introduced into the cells using methods well known in the art (see below). Integration of the linear fragments into the genome and the disruption of the gene can be determined based on the selection marker and can be verified by, for example, PCR or Southern blot analysis. Subsequent to its use in selection, a selectable marker can be removed from the genome of the host cell by, e.g., Cre-LoxP systems (see, e.g., Gossen et al., 2002, Ann. Rev. Genetics 36:153-173 and U.S. 2006/0014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene.

As used herein, the terms “variant” and “mutant” are used to describe a protein sequence that has been modified at one or more amino acids, compared to the wild-type sequence of a particular protein.

The terms “chimera,” “fusion polypeptide,” “fusion protein,” “fusion enzyme,” “fusion construct,” “chimeric protein,” “chimeric polypeptide,” “chimeric construct,” and “chimeric enzyme” can be used interchangeably herein to refer to proteins engineered through the joining of two or more genes that code for different proteins. Non-limiting examples of chimeric proteins include ACT1-3A (SEQ ID NO:8), ACT1-3B (SEQ ID NO:24), and ACT1-4 (SEQ ID NO:9). In some embodiments, a nucleic acid sequence encoding a polypeptide can include a tag sequence that encodes a “tag” designed to facilitate subsequent manipulation (e.g., to facilitate purification or detection), secretion, or localization of the encoded polypeptide. Tag sequences can be inserted in the nucleic acid sequence encoding the polypeptide such that the encoded tag is located at either the carboxyl or amino terminus of the polypeptide. Non-limiting examples of encoded tags include green fluorescent protein (GFP), human influenza hemagglutinin (HA), glutathione S transferase (GST), polyhistidine-tag (HIS tag), and Flag™ tag (Kodak, New Haven, Conn.). Other examples of tags include a chloroplast transit peptide, a mitochondrial transit peptide, an amyloplast peptide, signal peptide, or a secretion tag.

In some embodiments, a fusion protein is a protein altered by domain swapping. As used herein, the term “domain swapping” is used to describe the process of replacing a domain of a first protein with a domain of a second protein. In some embodiments, the domain of the first protein and the domain of the second protein are functionally identical or functionally similar. In some embodiments, the structure and/or sequence of the domain of the second protein differs from the structure and/or sequence of the domain of the first protein.

As used herein, the term “inactive fragment” is a fragment of the gene that encodes a protein having, e.g., less than about 10% (e.g., less than about 9%, less than about 8%, less than about 7%, less than about 6%, less than about 5%, less than about 4%, less than about 3%, less than about 2%, less than about 1%, or 0%) of the activity of the protein produced from the full-length coding sequence of the gene. Such a portion of a gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene with inactivation thereof.

As used herein, the terms “detectable amount,” “detectable concentration,” “measurable amount,” and “measurable concentration” refer to a level of acetylated 13R-MO and/or acetylated oxidized 13R-MO measured in AUC, μM/OD₆₀₀, mg/L, μM, or mM. Acetylated 13R-MO and/or acetylated oxidized 13R-MO production (i.e., total, supernatant, and/or intracellular levels) can be detected and/or analyzed by techniques generally available to one skilled in the art, for example, but not limited to, liquid chromatography-mass spectrometry (LC-MS), thin layer chromatography (TLC), high-performance liquid chromatography (HPLC), ultraviolet-visible spectroscopy/ spectrophotometry (UV-Vis), mass spectrometry (MS), and nuclear magnetic resonance spectroscopy (NMR). As used herein, the term “undetectable concentration” refers to a level of a compound that is too low to be measured and/or analyzed by techniques such as TLC, HPLC, UV-Vis, MS, or NMR. In some embodiments, a compound of an “undetectable concentration” is not present in an acetylated 13R-MO and/or acetylated oxidized 13R-MO composition.

Acetylated 13R-MO and/or acetylated oxidized 13R-MO can be isolated using a method described herein. For example, following fermentation, a culture broth can be centrifuged for 30 min at 7000 rpm at 4° C. to remove cells, or cells can be removed by filtration. The cell-free lysate can be obtained, for example, by mechanical disruption or enzymatic disruption of the host cells and additional centrifugation to remove cell debris. Mechanical disruption of the dried broth materials can also be performed, such as by sonication. The dissolved or suspended broth materials can be filtered using a micron or sub-micron prior to further purification, such as by preparative chromatography. The fermentation media or cell-free lysate can optionally be treated to remove low molecular weight compounds such as salt; and can optionally be dried prior to purification and re-dissolved in a mixture of water and solvent. The supernatant or cell-free lysate can be purified as follows: a column can be filled with, for example, HP20 Diaion resin (aromatic type Synthetic Adsorbent; Supelco) or other suitable non-polar adsorbent or reverse phase chromatography resin, and an aliquot of supernatant or cell-free lysate can be loaded on to the column and washed with water to remove the hydrophilic components. The acetylated 13R-MO and/or acetylated oxidized 13R-MO product can be eluted by stepwise incremental increases in the solvent concentration in water or a gradient from). The levels of acetylated 13R-MO and/or acetylated oxidized 13R-MO in each fraction, including the flow-through, can then be analyzed by LC-MS. Fractions can then be combined and reduced in volume using a vacuum evaporator. Additional purification steps can be utilized, if desired, such as additional chromatography steps and crystallization.

As used herein, the terms “or” and “and/or” is utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” In some embodiments, “and/or” is used to refer to the exogenous nucleic acids that a recombinant cell comprises, wherein a recombinant cell comprises one or more exogenous nucleic acids selected from a group. In some embodiments, “and/or” is used to refer to production of acetylated 13R-MO and/or acetylated oxidized 13R-MO. In some embodiments, “and/or” is used to refer to production of acetylated 13R-MO and/or acetylated oxidized 13R-MO. In some embodiments, “and/or” is used to refer to production of acetylated 13R-MO and/or acetylated oxidized 13R-MO, wherein acetylated 13R-MO and/or acetylated oxidized 13R-MO are produced through one or more of the following steps: culturing a recombinant microorganism, synthesizing acetylated 13R-MO and/or acetylated oxidized 13R-MO in a recombinant microorganism, and/or isolating acetylated 13R-MO and/or acetylated oxidized 13R-MO.

As used herein, the term “diterpene” is used to refer to a compound derived or prepared from four isoprene units. A diterpene according to the invention is a C₂₀-molecule comprising 20 carbon atoms. A diterpene typically comprises one or more ring structures, such as one or more monocyclic, bicyclic, tricyclic, or tetracyclic ring structure(s). The diterpene can comprise one or more double bonds. The diterpene can comprise up to three oxygen atoms, wherein the oxygen atom is generally present in the form of hydroxyl groups or part of a ring structure.

The term “substituted with a moiety” as used herein in relation to chemical compounds refers to hydrogen group(s) being substituted with the moiety. “Alkyl” as used herein refers to a saturated, straight, or branched hydrocarbon chain. The hydrocarbon chain preferably comprises from one to eighteen carbon atoms (C₁₋₁₈-alkyl), such as from one to six carbon atoms (C₁₋₆-alkyl), including methyl, ethyl, propyl, isopropyl, butyl, isobutyl, secondary butyl, tertiary butyl, pentyl, isopentyl, neopentyl, tertiary pentyl, hexyl, and isohexyl. In some embodiments, alkyl represents a C₁₋₃-alkyl group, which can in particular include methyl, ethyl, propyl, or isopropyl. The term “oxo” as used herein refers to a “═O” substituent. The term “keto” as used herein is used as a prefix to indicate presence of a carbonyl (C═O) group. The term “hydroxyl” as used herein refers to an “—OH” substituent. The term “acetylated” refers to presence of a CH₃O group.

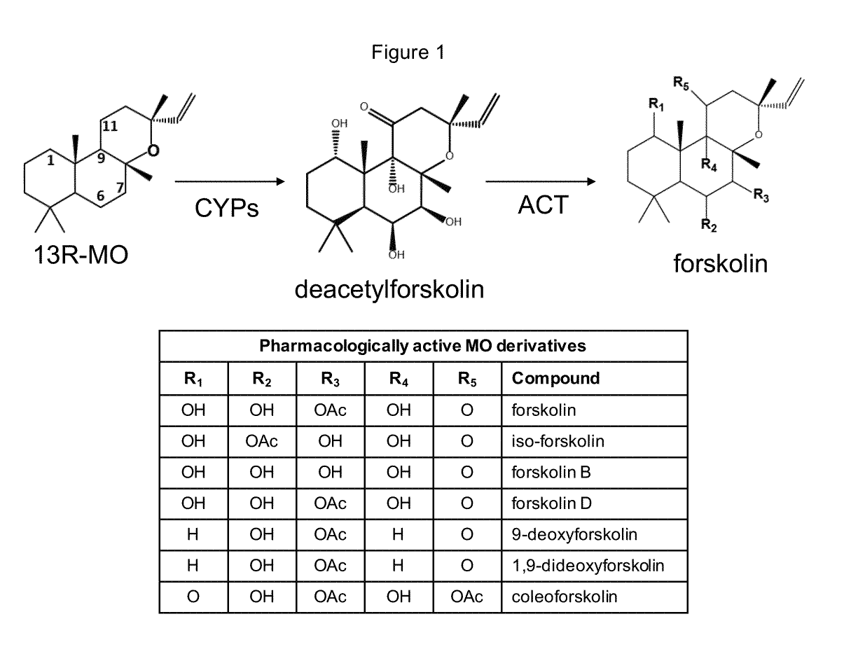

The abbreviation “13R-MO” as used herein refers to 13R-manoyl oxide, the structure of which is provided in FIG. 1. The structure also provides the numbering of the carbon atoms of the ring structure used herein. The term “oxidized 13R-MO” as used herein refers to 13R-MO substituted at one or more positions with an ═O and/or —OH group. The term “acetylated 13R-MO” as used herein refers to 13R-MO substituted with at least one acetyl group. The term “acetylated oxidized 13R-MO” as used herein refers to 13R-MO substituted with at least one acetyl group with one or more —OH and/or ═O groups.

Formulas for exemplary 13R-MO-derived compounds are shown in FIG. 1. For example, R₁ can be an —OH, —H, or ═O group. R₂ can be an —H, —OH, or acetyl group. R₃ can be an —H, —OH or acetyl group. R₄ can be an —H or —OH group. R₅ can be an —H, —OH, ═O, or acetyl group.

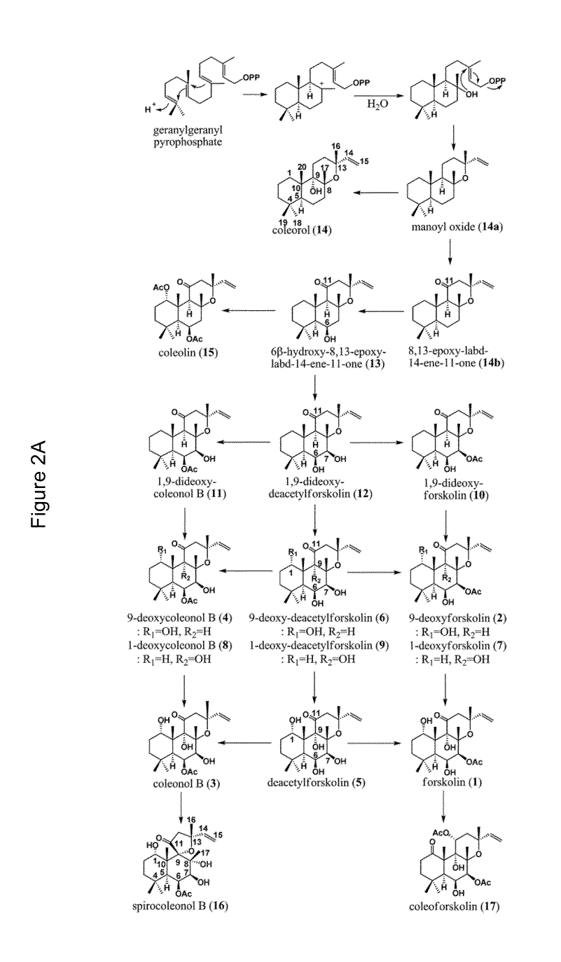

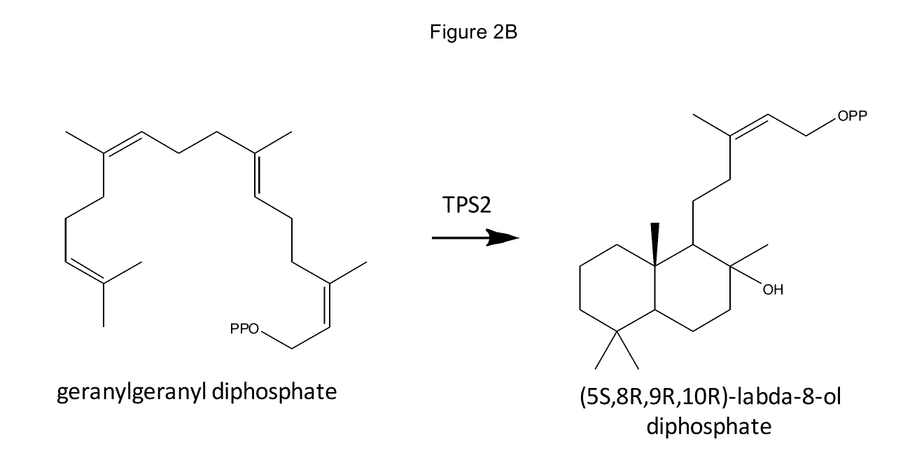

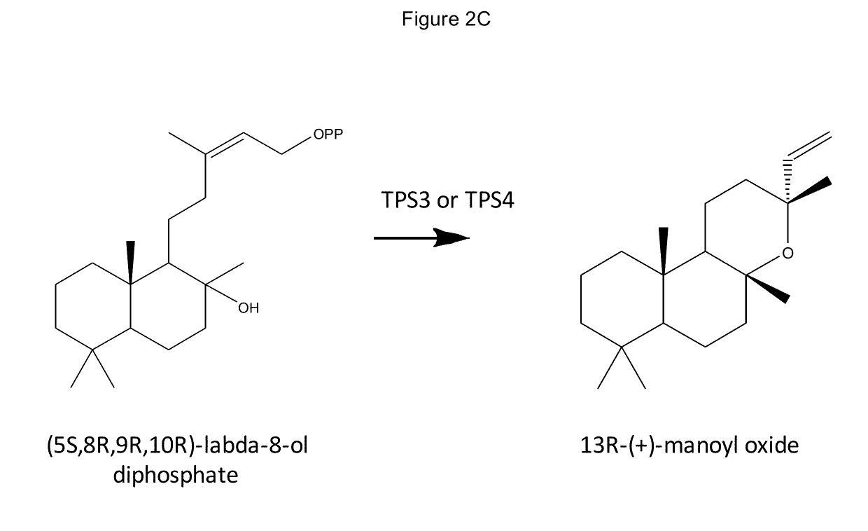

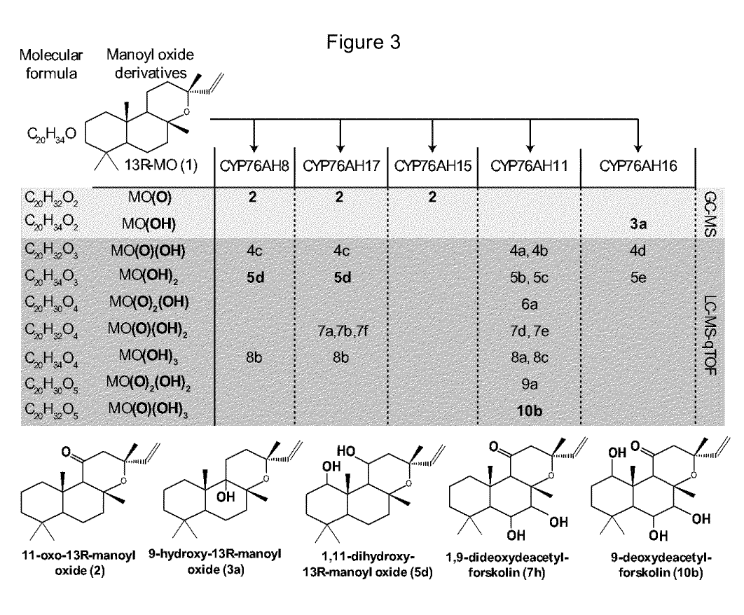

Oxidized 13R-MO may also be any of the oxidized 13R-MO compounds shown in FIG. 1, FIG. 2A, or FIG. 3. In particular, oxidized 13R-MO can be forskolin B or deacetylforskolin.

In some embodiments, acetylated 13R-MO is 13R-MO substituted at one or more of the positions 1, 6, 7, 9, and/or 11 with an acetyl group. In particular, acetylated 13R-MO according to the present invention can be 13R-MO substituted at one of the positions 1, 6, 7, 9, and/or 11 with an acetyl group. For example, acetylated 13R-MO according to the present invention can be 13R-MO substituted at position 7 with an acetyl group.

In some embodiments, acetylated oxidized 13R-MO is substituted with an acetyl group and is substituted with one or more —OH and/or ═O groups. In some embodiments, acetylated oxidized 13R-MO is 13R-MO substituted at one or more of the positions 1, 6, 7, 9, and/or 11, wherein at least one position is substituted with an acetyl group and at least one position is substituted with an —OH or ═O group. In particular, acetylated oxidized 13R-MO according to the present invention can be 13R-MO substituted at one of the positions 1, 6, 7, 9, and/or 11 with an acetyl group and at one or more of the positions 1, 6, 7, 9 and/or 11 with an —OH and/or ═O group. For example, acetylated oxidized 13R-MO according to the present invention can be 13R-MO substituted at one of the positions 1, 6, 7, and/or 9 with an acetyl group, at position 11 with an ═O group, and at one or more of the positions 1, 6, 7, and/or 9 with an —OH group. In another example, acetylated oxidized 13R-MO according to the present invention can be 13R-MO substituted at position 7 with an acetyl group and substituted at one or more of the positions 1, 6, 9, and/or 11 with an —OH and/or ═O group. In some embodiments, acetylated oxidized 13R-MO can be any of compounds 1, 3, 5, 7, 8, 9, or 14 shown in FIG. 2. In particular, acetylated oxidized 13R-MO can be forskolin, iso-forskolin, forskolin B, forskolin D, or coleoforskolin; formulas for these structures are provided in FIG. 1.

As used herein, the term “derivative” is used to refer to a compound produced from or capable of being produced (e.g., derived) from a similar compound. Non-limiting examples of 13R-MO derivatives include acetylated 13R-MO compounds, oxidized 13R-MO compounds, and acetylated oxidized 13R-MO compounds. For example, 13R-MO derivatives include forskolin, iso-forskolin, forskolin B, forskolin D, 9-deoxyforskolin, 1,9-dideoxyforskolin, and coleoforskolin. Additional 13R-MO derivatives are shown in FIG. 2.

As described herein, forskolin is a complex functionalized derivative of 13R-MO requiring region- and stereospecific oxidation of five carbon positions: one double-oxidation leading to a ketone and four single oxidation reactions yielding hydroxyl groups. The results presented herein show identification of diterpene synthases, cytochrome P450 mono-oxygenases, and acetyltransferases, which when co-expressed, result in production of forskolin.

### Diterpene Synthase (TPS)

In some embodiments, a host cell disclosed herein can comprise a diterpene synthase. The diterpene synthase (diTPS or TPS) can be from class II or class I, and in particular, be capable of converting geranylgeranyl diphosphate to (5S,8R,9R,10R)-labda-8-ol diphosphate and/or be capable of converting (5S,8R,9R,10R)-labda-8-ol diphosphate to 13R-MO. As described herein, 13R-MO is capable of being produced in a host cell comprising a gene encoding a terpene synthase polypeptide.

A diTPS of class II is an enzyme capable of catalyzing protonation-initiated cationic cycloisomerization of GGPP to form a diterpene pyrophosphate intermediate. The class II diTPS reaction can be terminated either by deprotonation or by water capture of the diphosphate carbocation. The diTPS of class II may in particular comprise the following motif of four amino acids: D/E-X-D-D, wherein X can be any amino acid, such as any naturally occurring amino acids. In particular, X can be an amino acid with a hydrophobic side chain, and thus, X can be A, I, L, M, F, W, Y, or V. Even more preferably, X is an amino acid with a small hydrophobic side chain, and thus X can be A, I, L, or V.

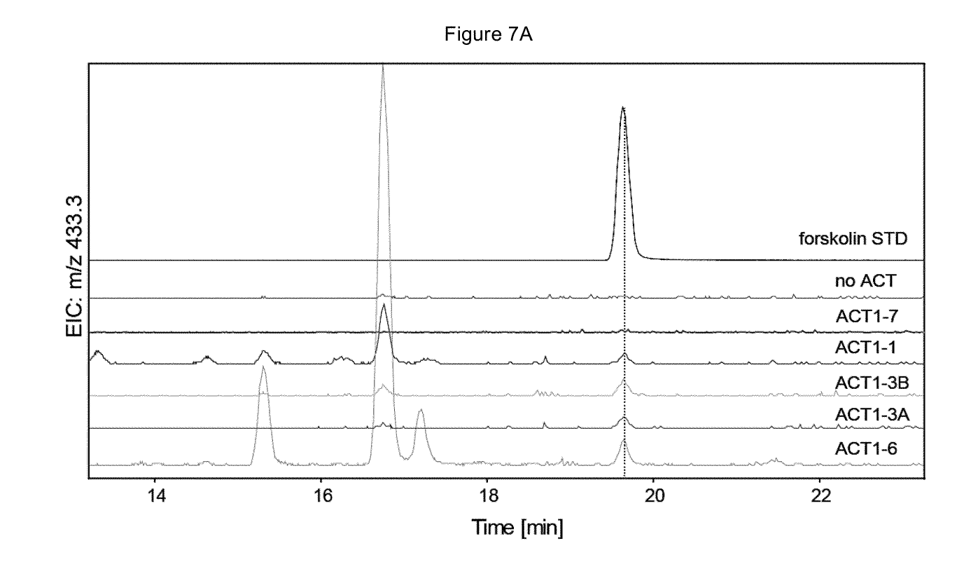

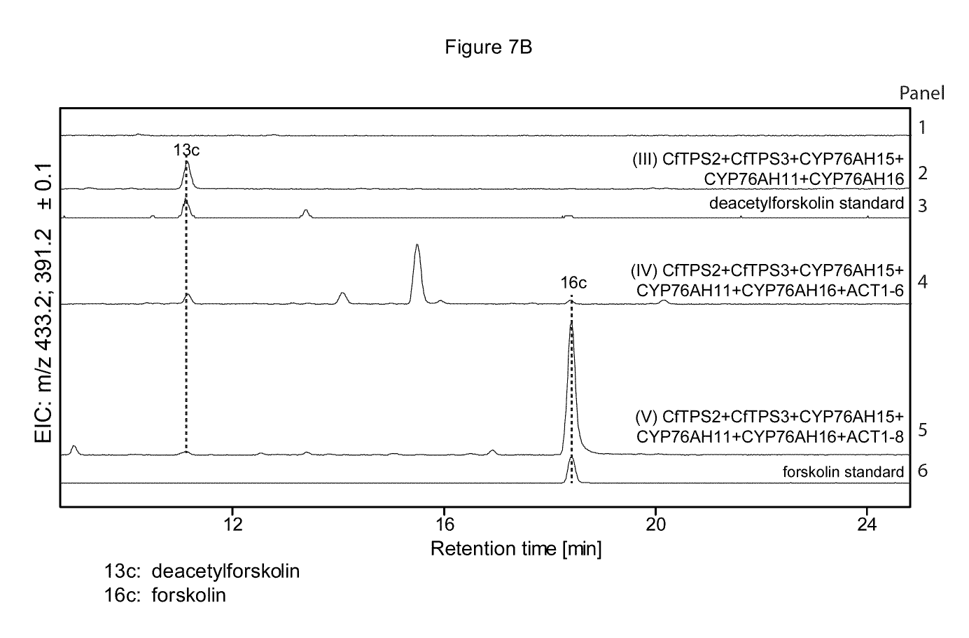

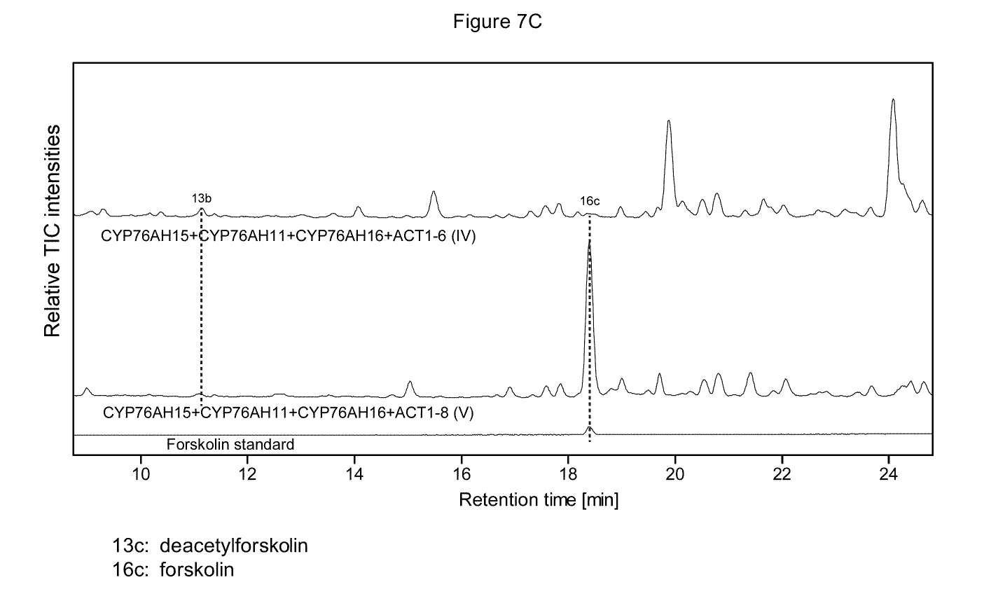

In embodiments of the invention relating to production of acetylated 13R-MO and/or acetylated oxidized 13R-MO, then it is preferred that the host organism comprises a gene encoding a TPS2 polypeptide. TPS2 catalyzes the reaction shown in FIG. 2B, wherein -OPP refers to diphosphate. In particular, it is preferred that the TPS2 is TPS2 of C. forskohlii. In particular, the TPS2 can be a polypeptide of SEQ ID NO:16 or a functional homolog thereof sharing at least 50% sequence identity therewith. TPS2 of SEQ ID NO:16 can be encoded by the nucleotide sequence set forth in SEQ ID NO:35. See Examples 1-4 and FIGS. 2B and 7B.

A diTPS of class I is an enzyme capable of catalyzing cleavage of the diphosphate group of the diterpene pyrophosphate intermediate and additionally preferably also is capable of catalyzing cyclization and/or rearrangement reactions on the resulting carbocation. As with the class II diTPSs, deprotonation or water capture may terminate the class I diTPS reaction leading to hydroxylation of the diterpene pyrophosphate intermediate.

A diTPS of class I may comprise the following motif of five amino acids: D-D-X-X-D/E, wherein X can be any amino acid, such as any naturally occurring amino acids. In particular, X can be an amino acid with a hydrophobic side chain, and thus X can for example be A, I, L, M, F, W, V, or V. Even more preferably, X is an amino acid with a small hydrophobic side chain, and thus X can be A, I, L, or V.

In embodiments of the invention relating to production of acetylated 13R-MO and/or acetylated oxidized 13R-MO, then it is preferred that the host organism comprises a gene encoding a TPS3 polypeptide and/or a gene encoding a TPS4 polypeptide. Preferably the TPS3 or TPS4 is an enzyme capable of catalyzing the reaction shown in FIG. 2C. In particular, it is preferred that the TPS3 is TPS3 of C. forskohlii. In particular, the TPS3 can be a polypeptide of SEQ ID NO:17 or a functional homolog thereof sharing at least 50% sequence identity therewith. TPS3 of SEQ ID NO:17 can be encoded by the nucleotide sequence set forth in SEQ ID NO:36. In particular, it is preferred that the TPS4 is TPS4 of C. forskohlii. In particular, the TPS4 can be a polypeptide of SEQ ID NO:18 or a functional homolog thereof sharing at least 40% sequence identity therewith. See Examples 1-4 and FIGS. 2C and 7B.

### Cytochrome P450 (CYP)

In some embodiments, a host cell disclosed herein can comprise a nucleic acid encoding an enzyme capable of catalyzing oxidation of 13R-MO. In some aspects, the enzyme capable of catalyzing oxidation of 13R-MO is a cytochrome P450 (CYP) polypeptide. CYPs according to the present invention are enzymes capable of catalyzing oxidation reactions using NAD(P)H as electron donor. Preferred CYPs according to the present invention are hemoproteins capable of catalyzing oxidation reactions that utilize NADPH and/or NADH to reductively cleave atmospheric dioxygen to produce a functionalized organic substrate and a molecule of water. As described herein, a host cell comprising a gene encoding a diterpene synthase polypeptide and genes encoding a CYP polypeptide is capable of producing oxidized 13R-MO.

CYPs are encoded by gene superfamily, which is divided into families sharing at least 40% sequence identity. The families are divided into subfamilies sharing at least 55% sequence identity. The CYP families have a number, which generally is written after “CYP,” Thus, by way of example, CYPs of family 74 are named CYP74. The subfamilies are indicated by a capital letter after the family number. Thus by way of example a CYP of family 74 and subfamily A is named CYP74A. Additional description of CYPs, the structural characteristics and the nomenclature thereof may for example be found in Schuler et al., Annu Rev. Plant Biol., 54:629-67 (2003) and in Podust et al., Nat. Prod. Rep., 29:1251-1266 (2012). Thus, the CYP to be used with the present invention can be a CYP as described in any of these references.

The CYP may comprise the following motif of five amino acids: NG-G-X-X-T/S, wherein X can be any amino acid, such as any naturally occurring amino acids. In particular, one of the X amino acids can be an amino acid with a charged side chain, and in particular an acidic side chain, such as E. A/G indicates that the amino acid can be A or G. Similarly, T/S indicates that the amino acid can be T or S. The CYP can also comprise the following motif 4 amino acids: E-X-X-R, wherein X can be any amino acid, such as any naturally occurring amino acids. In particular, X can be an amino acid with an uncharged side chain, such as an hydrophobic side chain. Furthermore, the CYP can comprise the following motif following motif of 10 amino acids: F-X-X-G-X-X-X-C-X-G, wherein X can be any amino acid, such as any naturally occurring amino acid. Furthermore, the CYP can comprise the following motif of 3 amino acids: P-F-G.

Preferably, the CYP is an enzyme capable of catalyzing one or more of the following reactions: a) conversion of 13R-MO to hydroxyl-13R-MO; b) conversion of hydroxyl-13R-MO to dihydroxy-13R-MO; c) conversion of hydroxyl-13R-MO to 13R-MO ketone; and/or d) conversion of hydroxyl-13R-MO to 13R-MO aldehyde.

It is preferred that the host organism comprises a gene encoding an enzyme capable of catalyzing oxidation of 13R-MO and/or of oxidized 13R-MO. Thus, the GYP may preferably be an enzyme capable of catalyzing oxidation of 13R-MO and/or of oxidized 13R-MO.

In one embodiment, a host organism comprises: a) a gene encoding GYP polypeptide capable of catalyzing hydroxylation of 13R-MO and/or of oxidized 13R-MO at the 1 position; b) a gene encoding CYP polypeptide capable of catalyzing hydroxylation of 13R-MO and/or of oxidized 13R-MO at the 6 position; c) a gene encoding GYP polypeptide capable of catalyzing hydroxylation of 13R-MO and/or of oxidized 13R-MO at the 7 position; d) a gene encoding CYP polypeptide capable of catalyzing hydroxylation of 13R-MO and/or of oxidized 13R-MO at the 9 position; and/or e) a gene encoding GYP polypeptide capable of catalyzing oxidation of 13R-MO and/or of oxidized 13R-MO at the 11 position to a ketone.

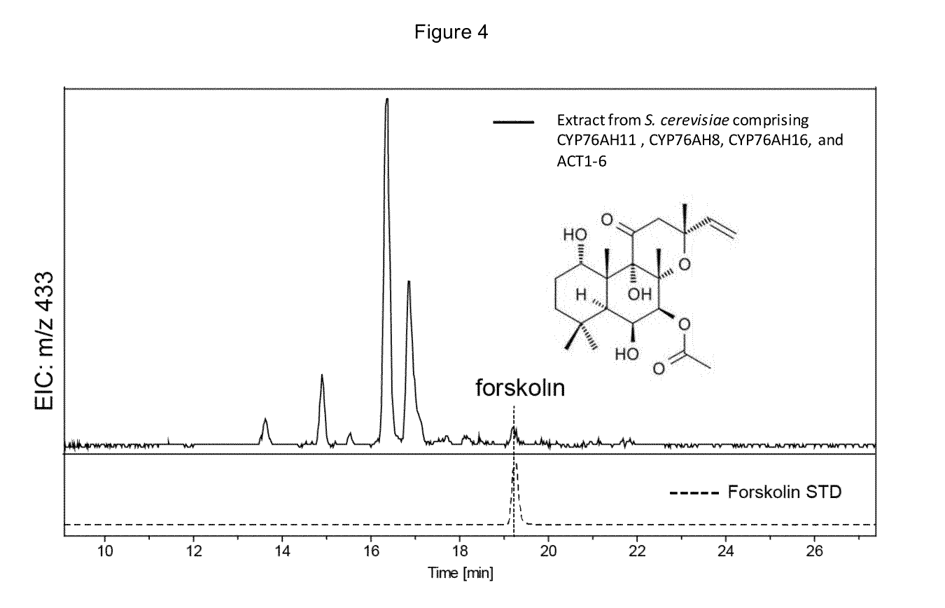

In some embodiments, the host organism comprises a gene encoding CYP76AH16. The CYP76AH16 may in particular be CYP76AH16 of SEQ ID NO:19 or a functional homolog thereof sharing at least 55% sequence identity therewith. Preferably, a functional homolog of CYP76AH16 is a polypeptide sharing above-mentioned sequence identity with CYP76AH16 and which also is capable of catalyzing hydroxylation of 13R-MO and/or of oxidized 13R-MO at the 9 position. See Examples 1-4 and FIGS. 4, 5, 6A, 6B, 7B, and 7C.

In some embodiments, the host organism comprises a gene encoding CYP76AH8. The CYP76AH8 may in particular be CYP76AH8 of SEQ ID NO:20 or a functional homolog thereof sharing at least 50% sequence identity therewith. See Examples 1-3 and FIGS. 4, 5, 6A, and 6B.

In some embodiments, the host organism comprises a gene encoding CYP76AH15. The CYP76AH15 may in particular be CYP76AH15 of SEQ ID NO:22 or a functional homolog thereof sharing at least 50% sequence identity therewith. See Examples 3 and 4 and FIGS. 7B and 7C.

In some embodiments, the host organism comprises a gene encoding CYP76AH17. The CYP76AH17 may in particular be CYP76AH17 of SEQ ID NO:23 or a functional homolog thereof sharing at least 50% sequence identity therewith.

In some embodiments, the host organism comprises a gene encoding CYP76AH11. The CYP76AH11 may in particular be CYP76AH11 of SEQ ID NO:21 or a functional homolog thereof sharing at least 50% sequence identity therewith. See Examples 1-4 and FIGS. 4, 5, 6A, 6B, 7B, and 7C.

Preferably, a functional homolog of CYP76AH8, CYP76AH15, CYP76AH17, or CYP76AH11 is a polypeptide sharing above-mentioned sequence identity with CYP76AH8, CYP76AH15, CYP76AH17, or CYP76AH11 and which also is capable of catalyzing hydroxylation of 13R-MO and/or of oxidized 13R-MO at the 1, 6, or 7 position or oxidation of 13R-MO at the 11 position.

### Diterpene Acetyltransferase (ACT)

In some embodiments, a host cell disclosed herein can comprise a nucleic acid encoding a diterpene acetyltransferase capable of catalyzing acetylation of 13R-MO and/or acetylation of oxidized 13R-MO. As described herein, a host cell comprising a gene encoding a diterpene synthase polypeptide, a gene encoding a CYP polypeptide, and a gene an ACT polypeptide is capable of producing acetylated oxidized 13R-MO, such as forskolin.

In some embodiments, a host cell disclosed herein comprises the diterpene acetyltransferase, ACT1-6. In some aspects, ACT1-6 is derived from C. forskohlii. In particular, the diterpene acetyltransferase can be ACT1-6 of SEQ ID NO:6 or a functional homolog thereof sharing at least 55% sequence identity therewith. In some embodiments, a functional homolog of ACT1-6 of SEQ ID NO:6 is a polypeptide sharing at least 90% sequence identity therewith. In some aspects, ACT1-6 of SEQ ID NO:6 is encoded by the nucleic acid set forth in SEQ ID NO:1 or SEQ ID NO:11, wherein SEQ ID NO:11 is optimized for expression in S. cerevisiae. See Examples 1 and 3 and FIGS. 4, 5, 7B, and 7C.

In some embodiments, a host cell disclosed herein comprises the diterpene acetyltransferase, ACT1-7. In some aspects, ACT1-7 is derived from C. forskohlii. In particular, the diterpene acetyltransferase can be ACT1-7 of SEQ ID NO:7 or a functional homolog thereof sharing at least 55% sequence identity therewith. In some embodiments, a functional homolog of ACT1-7 of SEQ ID NO:7 is a polypeptide sharing at least 90% sequence identity therewith. In some aspects, ACT1-7 of SEQ ID NO:7 is encoded by the nucleic acid set forth in SEQ ID NO:2 or SEQ ID NO:12, wherein SEQ ID NO:12 is optimized for expression in S. cerevisiae. See Examples 1 and 3 and FIG. 7A.

In some embodiments, a host cell disclosed herein comprises the diterpene acetyltransferase, ACT1-3A, including a host cell capable of producing forskolin. ACT1-3A can be derived from any suitable source; however, in a preferred embodiment, ACT1-3A is a synthetic protein. In particular, ACT1-3A can be a chimeric protein comprising sequences from two or more naturally occurring diterpene acetyltransferases. Thus, in one embodiment, ACT1-3A is a chimeric protein of sequences from different diterpene acetyltransferases from C. forskohlii. In a preferred embodiment of the invention, ACT1-3A can be engineered from ACT1-6 (SEQ ID NO:6) and ACT1-8 (SEQ ID NO:26) using PCR. In particular, the diterpene acetyltransferase can be ACT1-3A of SEQ ID NO:8 or a functional homolog thereof sharing at least 55% sequence identity therewith. In some embodiments, a functional homolog of ACT1-3A of SEQ ID NO:8 is a polypeptide sharing at least 90% sequence identity therewith. In some embodiments, ACT1-3A is encoded by the nucleic acid set forth in SEQ ID NO:3 or SEQ ID NO:13, wherein SEQ ID NO:13 is optimized for expression in S. cerevisiae. See Examples 1 and 3 and FIG. 7A.

In some embodiments, a host cell disclosed herein comprises the diterpene acetyltransferase, ACT1-3B, including a host cell capable of producing acetylated 13R-MO and/or acetylated oxidized 13R-MO, such as forskolin. ACT1-3B can be derived from any suitable source; however, in a preferred embodiment, ACT1-3B is a synthetic protein. In particular, ACT1-3B can be a chimeric protein comprising sequences from different naturally occurring diterpene acetyltransferases. Thus, in one embodiment, ACT1-3B is a chimeric protein of sequences from different diterpene acetyltransferases from C. forskohlii. In a preferred embodiment of the invention, ACT1-3B can be engineered from ACT1-6 (SEQ ID NO:6) and ACT1-8 (SEQ ID NO:26) using PCR. In particular, the diterpene acetyltransferase can be ACT1-3B of SEQ ID NO:24 or a functional homolog thereof sharing at least 55% sequence identity therewith. In some embodiments, a functional homolog of ACT1-3B of SEQ ID NO:24 is a polypeptide sharing at least 90% sequence identity therewith. In some embodiments, ACT1-3B of SEQ ID NO:24 is encoded by the nucleic acid of SEQ ID NO:25. See Examples 1 and 3 and FIG. 7A.

In some embodiments, a host cell disclosed herein comprises the diterpene acetyltransferase, ACT1-4, including a host cell capable of producing forskolin. ACT1-4 can be derived from any suitable source; however, in a preferred embodiment, ACT1-4 is a synthetic protein. In particular, ACT4 can be a chimeric protein comprising sequences from two or more naturally occurring diterpene acetyltransferases. Thus, in one embodiment, ACT1-4 is a chimeric protein of sequences from different diterpene acetyltransferases from C. forskohlii. In a preferred embodiment of the invention, ACT1-4 can be engineered from ACT1-6 (SEQ ID NO:6) and ACT1-8 (SEQ ID NO:26) using PCR. In particular, the diterpene acetyltransferase can be ACT1-4 of SEQ ID NO:9 or a functional homolog thereof sharing at least 55% sequence identity therewith. In some embodiments, a functional homolog of ACT1-4 of SEQ ID NO:9 is a polypeptide sharing at least 90% sequence identity therewith. In some embodiments, ACT1-4 is encoded by the nucleic acid set forth in SEQ ID NO:4 or SEQ ID NO:14, wherein SEQ ID NO:14 is optimized for expressing in S. cerevisiae. See Example 1.

In some embodiments, a host cell disclosed herein comprises the diterpene acetyltransferase, ACT1-1. ACT1-1 can be derived from any suitable source; however, in a preferred embodiment, ACT1-1 is derived from C. forskohlii. In particular, the diterpene acetyltransferase can be ACT1-1 of SEQ ID NO:10 or a functional homolog thereof sharing at least 55% sequence identity therewith. In some embodiments, a functional homolog of ACT1-1 of SEQ ID NO:10 is a polypeptide sharing at least 90% sequence identity therewith. In some embodiments, ACT1-4 is encoded by the nucleic acid set forth in SEQ ID NO:5 or SEQ ID NO:15, wherein SEQ ID NO:15 is optimized for expression in S. cerevisiae. See Examples 1 and 3 and FIG. 7A.

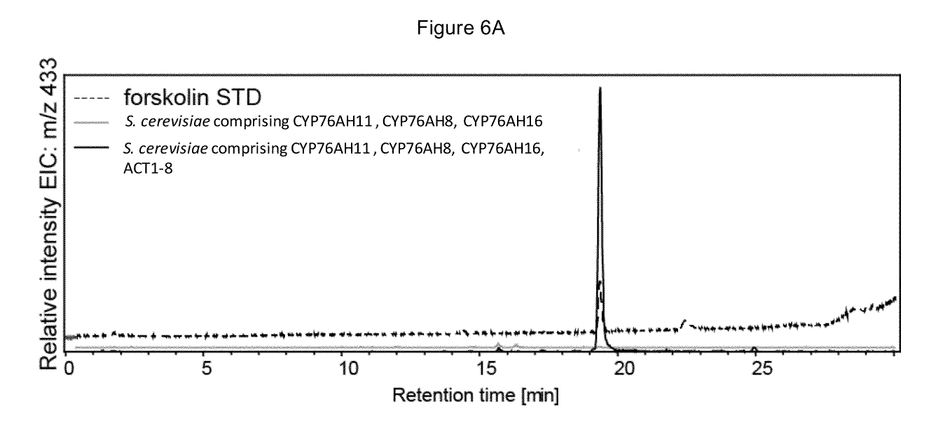

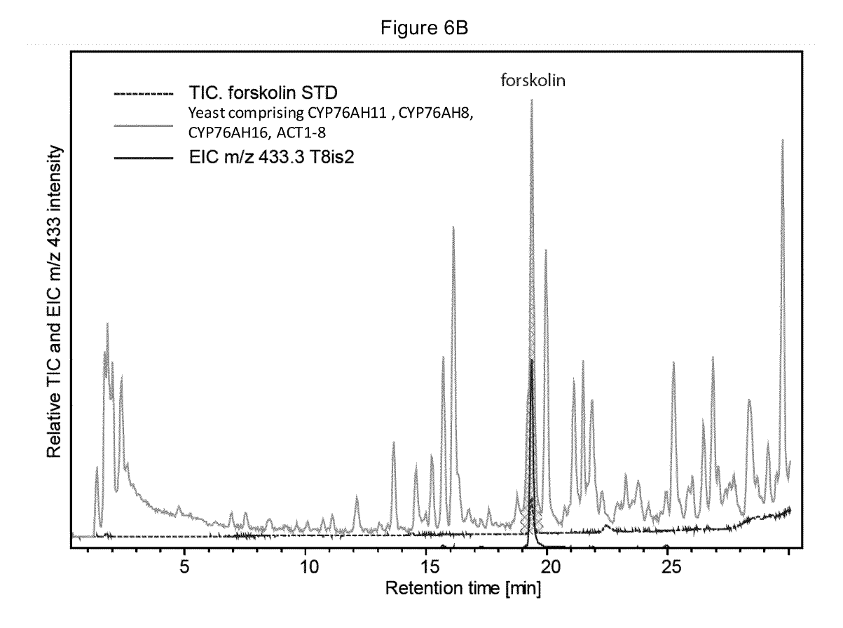

In some embodiments, a host cell disclosed herein comprises the diterpene acetyltransferase, ACT1-8. ACT1-1 can be derived from any suitable source; however, in a preferred embodiment, ACT1-8 is derived from C. forskohlii. In particular, the diterpene acetyltransferase can be ACT1-8 of SEQ ID NO:26 or a functional homolog thereof sharing at least 55% sequence identity therewith. In some embodiments, a functional homolog of ACT1-8 of SEQ ID NO:26 is a polypeptide sharing at least 90% sequence identity therewith. In some embodiments, ACT1-8 is encoded by the nucleic acid set forth in SEQ ID NO:27. See Examples 1-4 and FIGS. 6A, 6B, 7B, and 7C.

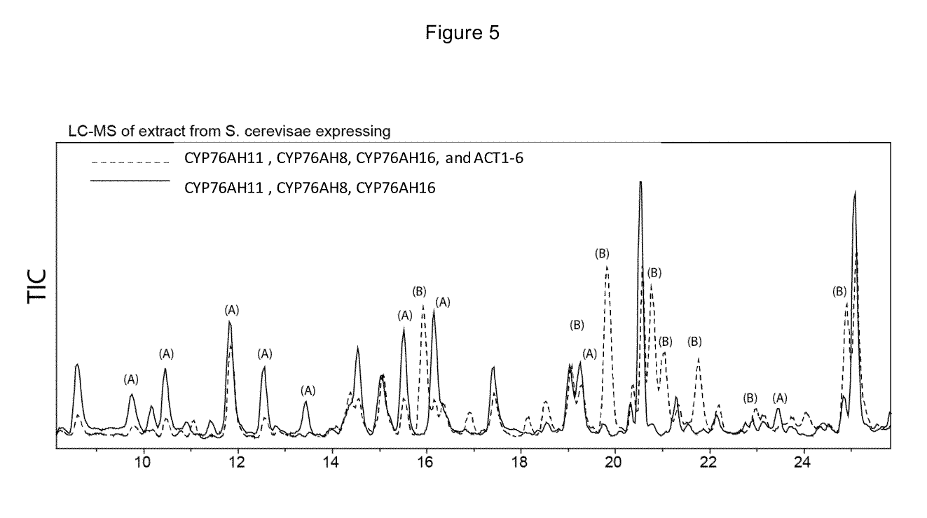

In some aspects, an S. cerevisiae strain comprising TPS2 (SEQ ID NO:35, SEQ ID NO:16), TPS3 (SEQ ID NO:36, SEQ ID NO:17), CYP76AH16 (SEQ ID NO:19), CYP76AH15 (SEQ ID NO:22), CYP76AH11 (SEQ ID NO:21), and either ACT1-3A (SEQ ID NO:13, SEQ ID NO:8), ACT1-4 (SEQ ID NO:14, SEQ ID NO:9), ACT1-6 (SEQ ID NO:11, SEQ ID NO:6), ACT1-7 (SEQ ID NO:12, SEQ ID NO:7), or ACT1-8 (SEQ ID NO:28, SEQ ID NO:26) produces forskolin. See Example 1 and FIGS. 4 and 5.

In some aspects, an S. cerevisiae strain comprising TPS2 (SEQ ID NO:35, SEQ ID NO:16), TPS3 (SEQ ID NO:36, SEQ ID NO:17), CYPAH16 (SEQ ID NO:19), CYPAH8 (SEQ ID NO:20), CYP76AH11 (SEQ ID NO:21), and ACT1-8 (SEQ ID NO:28, SEQ ID NO:26) produces forskolin and minute amounts of deacetylforskolin. See Example 2 and FIGS. 6A and 6B.

In some aspects, N. benthamiana plants comprising TPS2 (SEQ ID NO:16), TPS3 (SEQ ID NO:17), CYP76AH16 (SEQ ID NO:19), CYP76AH15 (SEQ ID NO:22), and CYP76AH11 (SEQ ID NO:21) produce deacetylforskolin. In some aspects, N. benthamiana plants comprising TPS2 (SEQ ID NO:16), TPS3 (SEQ ID NO:17), CYP76AH16 (SEQ ID NO:19), CYP76AH15 (SEQ ID NO:22), CYP76AH11 (SEQ ID NO:21), and either ACT1-6 (SEQ ID NO:1, SEQ ID NO:6), ACT1-3A (SEQ ID NO:3, SEQ ID NO:8), ACT1-3B (SEQ ID NO:25, SEQ ID NO:24), or ACT1-1 (SEQ ID NO:5, SEQ ID NO:10) produce forskolin. In some aspects, N. benthamiana plants comprising C. forskohlii DXS (SEQ ID NO:29, SEQ ID NO:30), C. forskohlii GGPPS (SEQ ID NO:31, SEQ ID NO:32), TPS2 (SEQ ID NO:16), TPS3 (SEQ ID NO:17), CYP76AH16 (SEQ ID NO:19), CYP76AH15 (SEQ ID NO:22), CYP76AH11 (SEQ ID NO:21), and either ACT1-6 (SEQ ID NO:1, SEQ ID NO:6) or ACT1-8 (SEQ ID NO:27, SEQ ID NO:26) produce forskolin. See Example 3 and FIGS. 7A, 7B, and 7C.

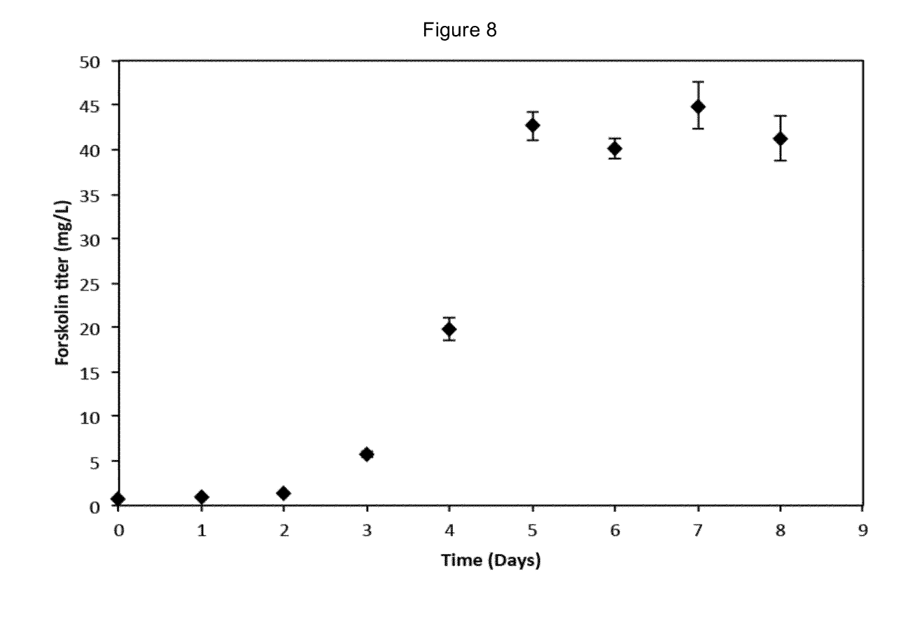

In some aspects, an S. cerevisiae strain comprising C. forskohlii POR (SEQ ID NO:33, SEQ ID NO:34), CYP76AH15 (SEQ ID NO:22), CYP76AH11 (SEQ ID NO:21), CYP76AH16 (SEQ ID NO:19), and ACT1-8 (SEQ ID NO:28, SEQ ID NO:26) produces forskolin by fermentation. Forskolin levels can accumulate to at least 40 mg/L. See Example 4 and FIG. 8.

### Functional Homologs

Functional homologs of the polypeptides described above are also suitable for use in producing acetylated 13R-MO and/or acetylated oxidized 13R-MO in a recombinant host. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of acetylated 13R-MO and/or acetylated oxidized 13R-MO biosynthesis polypeptides. Sequence analysis can involve BLAST, Reciprocal BLAST, or PSI-BLAST analysis of non-redundant databases using an amino acid sequence as the reference sequence. Amino acid sequence is, in some instances, deduced from the nucleotide sequence. Those polypeptides in the database that have greater than 40% sequence identity are candidates for further evaluation for suitability as a acetylated 13R-MO and/or acetylated oxidized 13R-MO biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in acetylated 13R-MO and/or acetylated oxidized 13R-MO biosynthesis polypeptides, e.g., conserved functional domains. In some embodiments, nucleic acids and polypeptides are identified from transcriptome data based on expression levels rather than by using BLAST analysis.

Conserved regions can be identified by locating a region within the primary amino acid sequence of an acetylated 13R-MO and/or acetylated oxidized 13R-MO biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27; 260-262 (1999). Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

Methods to modify the substrate specificity of a polypeptide are known to those skilled in the art, and include without limitation site-directed/rational mutagenesis approaches, random directed evolution approaches and combinations in which random mutagenesis/saturation techniques are performed near the active site of the enzyme. For example see Osmani of al., 2009, Phytochemistry 70: 325-347.

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between. A % identity for any candidate nucleic acid or polypeptide relative to a reference nucleic acid or polypeptide can be determined as follows. A reference sequence (e.g., a nucleic acid sequence or an amino acid sequence described herein) is aligned to one or more candidate sequences using the computer program Clustal Omega (version 1.2.1, default parameters), which allows alignments of nucleic acid or polypeptide sequences to be carried out across their entire length (global alignment). Chenna et al., 2003, Nucleic Acids Res. 31(13):3497-500.

Clustal Omega calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: %age; number of top diagonals; 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: %age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The Clustal Omega output is a sequence alignment that reflects the relationship between sequences. Clustal Omega can be run, for example, at the Baylor College of Medicine Search Launcher site on the World Wide Web (searchlauncher.bcm.tmc.edu/multi-align/multi-align.html) and at the European Bioinformatics Institute site at http://www.ebi.ac.uk/Tools/msa/clustalo/.

To determine a % identity of a candidate nucleic acid or amino acid sequence to a reference sequence, the sequences are aligned using Clustal Omega, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

Acetylated 13R-MO and/or Acetylated Oxidized 13R-MO Biosynthesis Nucleic Acids

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous gene. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic adds typically are integrated at positions other than the position where the native sequence is found. “Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

One or more genes can be combined in a recombinant nucleic acid construct in “modules” useful for a discrete aspect of acetylated 13R-MO and/or acetylated oxidized 13R-MO production. Combining a plurality of genes in a module, particularly a polycistronic module, facilitates the use of the module in a variety of species. For example, an acetylated 13R-MO and/or acetylated oxidized 13R-MO gene duster can be combined in a polycistronic module such that, after insertion of a suitable regulatory region, the module can be introduced into a wide variety of species. As another example, an acetylated 13R-MO and/or acetylated oxidized 13R-MO gene cluster can be combined such that each coding sequence is operably linked to a separate regulatory region, to form a module. Such a module can be used in those species for which monocistronic expression is necessary or desirable. In addition to genes useful for acetylated 13R-MO and/or acetylated oxidized 13R-MO production, a recombinant construct typically also comprises an origin of replication, and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

In some cases, it is desirable to inhibit one or more functions of an endogenous polypeptide in order to divert metabolic intermediates towards acetylated 13R-MO and/or acetylated oxidized 13R-MO biosynthesis. As another example, it may be desirable to inhibit degradative functions of certain endogenous gene products. In such cases, a nucleic acid that overexpresses the polypeptide or gene product may be included in a recombinant construct that is transformed into the strain. Alternatively, mutagenesis can be used to generate mutants in genes for which it is desired to increase or enhance function.

### Host Organisms

Recombinant hosts can be used to express polypeptides for the producing acetylated 13R-MO and/or acetylated oxidized 13R-MO, including mammalian, insect, plant, and algal cells. A number of prokaryotes and eukaryotes are also suitable for use in constructing the recombinant microorganisms described herein, e.g., gram-negative bacteria, yeast, and fungi. A species and strain selected for use as an acetylated 13R-MO and/or acetylated oxidized 13R-MO production strain is first analyzed to determine which production genes are endogenous to the strain and which genes are not present. Genes for which an endogenous counterpart is not present in the strain are advantageously assembled in one or more recombinant constructs, which are then transformed into the strain in order to supply the missing function(s).

Typically, the recombinant microorganism is grown in a fermenter at a temperature(s) for a period of time, wherein the temperature and period of time facilitate the production of acetylated 13R-MO and/or acetylated oxidized 13R-MO. The constructed and genetically engineered microorganisms provided by the invention can be cultivated using conventional fermentation processes, including, inter alia, chemostat, batch, fed-batch cultivations, semi-continuous fermentations such as draw and fill, continuous perfusion fermentation, and continuous perfusion cell culture. Depending on the particular microorganism used in the method, other recombinant genes such as isopentenyl biosynthesis genes and terpene synthase and cyclase genes may also be present and expressed. Levels of substrates and intermediates can be determined by extracting samples from culture media for analysis according to published methods.

Carbon sources of use in the instant method include any molecule that can be metabolized by the recombinant host cell to facilitate growth and/or production of the acetylated 13R-MO and/or acetylated oxidized 13R-MO. Examples of suitable carbon sources include, but are not limited to, sucrose (e.g., as found in molasses), fructose, xylose, ethanol, glycerol, glucose, cellulose, starch, cellobiose or other glucose-comprising polymer. In embodiments employing yeast as a host, for example, carbons sources such as sucrose, fructose, xylose, ethanol, glycerol, and glucose are suitable. The carbon source can be provided to the host organism throughout the cultivation period or alternatively, the organism can be grown for a period of time in the presence of another energy source, e.g., protein, and then provided with a source of carbon only during the fed-batch phase.

After the recombinant microorganism has been grown in culture for the period of time, wherein the temperature and period of time facilitate the production of acetylated 13R-MO and/or acetylated oxidized 13R-MO can then be recovered from the culture using various techniques known in the art. In some embodiments, a permeabilizing agent can be added to aid the feedstock entering into the host and product getting out. For example, a crude lysate of the cultured microorganism can be centrifuged to obtain a supernatant. The resulting supernatant can then be applied to a chromatography column, e.g., a C-18 column, and washed with water to remove hydrophilic compounds, followed by elution of the compound(s) of interest with a solvent such as methanol. The compound(s) can then be further purified by preparative HPLC. See also, WO 2009/140394.

It will be appreciated that the various genes and modules discussed herein can be present in two or more recombinant hosts rather than a single host. When a plurality of recombinant hosts is used, they can be grown in a mixed culture to accumulate acetylated 13R-MO and/or acetylated oxidized 13R-MO.

Alternatively, the two or more hosts each can be grown in a separate culture medium and the product of the first culture medium can be introduced into second culture medium to be converted into a subsequent intermediate or into an end product. The product produced by the second or final host is then recovered. It will also be appreciated that in some embodiments, a recombinant host is grown using nutrient sources other than a culture medium and utilizing a system other than a fermenter.

Exemplary prokaryotic and eukaryotic species are described in more detail below. However, it will be appreciated that other species can be suitable. For example, suitable species can be in a genus such as Agaricus, Aspergillus, Bacillus, Candida, Corynebacterium, Eremothecium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Phanerochaete chrysosporium, Pichia pastoris, Cyberlindnera jadinii, Physcomitrella patens, Rhodoturula glutinis, Rhodoturula mucilaginosa, Phaffia rhodozyma, Xanthophyllomyces dendrorhous, Fusarium fujikuroi/Gibberella fujikuroi, Candida utilis, Candida glabrata, Candida albicans, and Yarrowia lipolytica.

In some embodiments, a microorganism can be a prokaryote such as Escherichia bacteria cells, for example, Escherichia coli cells; Lactobacillus bacteria cells; Lactococcus bacteria cells; Cornebacterium bacteria cells; Acetobacter bacteria cells; Acinetobacter bacteria cells; or Pseudomonas bacterial cells.

In some embodiments, a microorganism can be an Ascomycete such as Gibberella fujikuroi, Kluyveromyces lactis, Schizosaccharomyces pompe, Aspergillus niger, Yarrowia lipolytica, Ashbya gossypii, or S. cerevisiae.

In some embodiments, a microorganism can be an algal cell such as Blakeslea trispora, Dunaliella salina, Haematococcus pluvialis, Chlorella sp., Undaria pinnatifida, Sargassum, Laminaria japonica, Scenedesmus almeriensis species.

In some embodiments, a microorganism can be a cyanobacterial cell such as Blakeslea trispora, Dunaliella salina, Haematococcus pluvialis, Chlorella sp., Undaria pinnatifida, Sargassum, Laminaria japonica, Scenedesmus almeriensis.

### Saccharomyces spp.

Saccharomyces is a widely used chassis organism in synthetic biology, and can be used as the recombinant microorganism platform. For example, there are libraries of mutants, plasmids, detailed computer models of metabolism and other information available for S. cerevisiae, allowing for rational design of various modules to enhance product yield. Methods are known for making recombinant microorganisms.

### Aspergillus spp.

Aspergillus species such as A. oryzae, A. niger and A. sojae are widely used microorganisms in food production and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for producing acetylated 13R MO and/or acetylated oxidized 13R-MO.

E. coli

E. coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

### Agaricus, Gibberella, and Phanerochaete spp.

Agaricus, Gibberella, and Phanerochaete spp. can be useful because they are known to produce large amounts of isoprenoids in culture. Thus, the terpene precursors for producing large amounts of acetylated 13R-MO and/or acetylated oxidized 13R-MO are already produced by endogenous genes. Thus, modules comprising recombinant genes for acetylated 13R-MO and/or acetylated oxidized 13R-MO biosynthesis polypeptides can be introduced into species from such genera without the necessity of introducing mevalonate or MEP pathway genes.

Arxula adeninivorans (Blastobotrys adeninivorans)

Arxula adeninivorans is dimorphic yeast (it grows as budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Yarrowia lipolytica

Yarrowia lipolytica is dimorphic yeast (see Arxula adeninivorans) and belongs to the family Hemiascomycetes. The entire genome of Yarrowia lipolytica is known. Yarrowia species is aerobic and considered to be non-pathogenic. Yarrowia is efficient in using hydrophobic substrates (e.g., alkanes, fatty acids, oils) and can grow on sugars. it has a high potential for industrial applications and is an oleaginous microorganism. Yarrowia lipolyptica can accumulate lipid content to approximately 40% of its dry cell weight and is a model organism for lipid accumulation and remobilization. See e.g., Nicaud, 2012, Yeast 29(10):409-18; Beopoulos et al., 2009, Biochimie 91(6):692-6; Banker et al., 2009, Appl Microbiol Biotechnol. 84(5):847-65.

### Rhodotorula sp.

Rhodotorula is unicellular, pigmented yeast. The oleaginous red yeast, Rhodotorula glutinis, has been shown to produce lipids and carotenoids from crude glycerol (Saenge et al., 2011, Process Biochemistry 46(1):210-8). Rhodotorula toruloides strains have been shown to be an efficient fed-batch fermentation system for improved biomass and lipid productivity (Li et al., 2007, Enzyme and Microbial Technology 41:312-7).

Rhodosporidium toruloides

Rhodosporidium toruloides is oleaginous yeast and useful for engineering lipid-production pathways (See e.g. Zhu et al., 2013, Nature Commun. 3:1112; Ageitos et al., 2011, Applied Microbiology and Biotechnology 90(4):1219-27).

Candida boidinii

Candida boidinii is methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH. See, e.g., Mattanovich et al., 2012, Methods Mol Biol. 824:329-58; Khoury et al., 2009, Protein Sci. 18(10):2125-38.

Hansenula polymorpha (Pichia angusta)

Hansenula polymorpha is methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes. See, e.g., Xu et al., 2014, Virol Sin. 29(6):403-9.

Kluyveromyces lactis

Kluyveromyces lactis is yeast regularly applied to the production of kefir. It can grow on several sugars, most importantly on lactose which is present in milk and whey. It has successfully been applied among others for producing chymosin (an enzyme that is usually present in the stomach of calves) for producing cheese. Production takes place in fermenters on a 40,000 L scale. See, e.g., van (doyen of at, 2006, FEMS Yeast Res. 6(3):381-92.

Pichia pastoris

Pichia pastoris is methylotrophic yeast (see Candida boidinii and Hansenula polymorpha). It provides an efficient platform for producing foreign proteins. Platform elements are available as a kit and it is worldwide used in academia for producing proteins. Strains have been engineered that can produce complex human N-glycan (yeast glycans are similar but not identical to those found in humans). See, e.g., Piirainen et al., 2014, N Biotechnol. 31(6):532-7.

### Physcomitrella spp.

Physcomitrella mosses, when grown in suspension culture, have characteristics similar to yeast or other fungal cultures. This genera can be used for producing plant secondary metabolites, which can be difficult to produce in other types of cells.

In some embodiments, the host organism is a plant. A plant or plant cell can be transformed by having a heterologous gene integrated into its genome, i.e., it can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a certain number of cell divisions. Both transiently transformed and stably transformed transgenic plants and plant cells can be useful in the methods described herein.

Plant cells comprising a heterologous gene used in methods described herein can constitute part or all of a whole plant. Such plants can be grown in a manner suitable for the species under consideration, either in a growth chamber, a greenhouse, or in a field. Plants may also be progeny of an initial plant comprising a heterologous gene provided the progeny inherits the heterologous gene. Seeds produced by a transgenic plant can be grown and then selfed (or outcrossed and selfed) to obtain seeds homozygous for the nucleic acid construct.

The plants to be used with the invention can be grown in suspension culture, or tissue or organ culture. For the purposes of this invention, solid and/or liquid tissue culture techniques can be used. When using solid medium, plant cells can be placed directly onto the medium or can be placed onto a filter that is then placed in contact with the medium. When using liquid medium, transgenic plant cells can be placed onto a flotation device, e.g., a porous membrane that contacts the liquid medium.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, U.S. Pat. Nos. 5,538,880; 5,204,253; 6,329,571; and 6,013,863. If a cell or cultured tissue is used as the recipient tissue for transformation, plants can be regenerated from transformed cultures if desired, by techniques known to those skilled in the art.

The plant comprising a heterologous nucleic acid to be used with the present invention can for example be: corn (Zea mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (Helianthus annuas), wheat (Tritium aestivum and other species), Triticale, Rye (Secale) soybean (Glycine max), tobacco (Nicotiana tabacum), potato (Solanum tuberosum), peanuts (Arachis hypogaea), cotton (Gossypium hirsutum), sweet potato (Impomoea batatus), cassava (Manihot esculenta), coffee (Coffea spp.), coconut (Cocos nucifera), pineapple (Anana comosus), citrus (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Primus amygdalus), apple (Malus spp.), Pear (Pyrus spp.), plum and cherry tree (Prunus spp.), Ribes (currant etc.), Vitis, Jerusalem artichoke (Helianthemum spp.), non-cereal grasses (Grass family), sugar and fodder beets (Beta vulgaris), chicory, oats, barley, vegetables, or ornamentals.

For example, plants of the present invention are crop plants (for example, cereals and pulses, maize, wheat, potatoes, tapioca, rice, sorghum, millet, cassava, barley, pea, sugar beets, sugar cane, soybean, oilseed rape, sunflower and other root, tuber or seed crops. Other important plants maybe fruit trees, crop trees, forest trees or plants grown for their use as spices or pharmaceutical products (Mentha spp., clove, Artemesia spp., Thymus spp., Lavendula spp., Allium spp., Hypericum, Catharanthus spp., Vinca spp., Papaver spp., Digitalis spp., Rawolfia spp., Vanilla spp., Petrusilium spp., Eucalyptus, tea tree, Picea spp., Pinus spp., Abies spp., Juniperus spp. Horticultural plants which can be used with the present invention may include lettuce, endive, and vegetable brassicas including cabbage, broccoli, and cauliflower, carrots, and carnations and geraniums.

The plant can also be tobacco, cucurbits, carrot, strawberry, sunflower, tomato, pepper, or Chrysanthemum.

The plant may also be a grain plants for example oil-seed plants or leguminous plants. Seeds of interest include grain seeds, such as corn, wheat, barley, sorghum, rye, etc. Oil-seed plants include cotton soybean, safflower, sunflower, Brassica, maize, alfalfa, palm, coconut, etc. Leguminous plants include beans and peas. Beans include guar, locust bean, fenugreek, soybean, garden beans, cowpea, mung bean, lima bean, fava bean, lentils, chickpea.

In a further embodiment of the invention the plant can be maize, rice, wheat, sugar beet, sugar cane, tobacco, oil seed rape, potato, soybean, or Arabidopsis thaliana. In some embodiments, the plant is not C. forskohlii.

TABLE 1
Sequence listing key.

| | |
|---|---|
| SEQ ID NO: 1 | cDNA encoding ACT1-6 from C. forskohlii |
| SEQ ID NO: 2 | cDNA encoding ACT1-7 from C. forskohlii |
| SEQ ID NO: 3 | cDNA encoding ACT1-3A |
| SEQ ID NO: 4 | cDNA encoding ACT1-4 from C. forskohlii |
| SEQ ID NO: 5 | cDNA encoding ACT1-1 from C. forskohlii |
| SEQ ID NO: 6 | Amino acid sequence of ACT1-6 from C. forskohlii |
| SEQ ID NO: 7 | Amino acid sequence of ACT1-7 from C. forskohlii |
| SEQ ID NO: 8 | Amino acid sequence of ACT1-3A |
| SEQ ID NO: 9 | Amino acid sequence of ACT1-4 |
| SEQ ID NO: 10 | Amono acid sequence of ACT1-1 from C. forskohlii |
| SEQ ID NO: 11 | DNA Sequence encoding ACT1-6 from C. forskohlii |
| | codon optimized for expression in yeast |
| SEQ ID NO: 12 | DNA Sequence encoding ACT1-7 C. forskohlii |
| | codon optimized for expression in yeast |
| SEQ ID NO: 13 | DNA Sequence encoding ACT1-3A codon optimized |
| | for expression in yeast |
| SEQ ID NO: 14 | DNA Sequence encoding ACT1-4 codon optimized |
| | for expression in yeast |
| SEQ ID NO: 15 | DNA Sequence encoding ACT1-1 from C. forskohlii |
| | codon optimized for expression in yeast |
| SEQ ID NO: 16 | Amino acid sequence of TPS2 from C. forskohlii; |
| | GenBank accession number KF444507 |
| SEQ ID NO: 17 | Amino acid sequence of TPS3 from C. forskohlii; |
| | GenBank accession number KF444508 |
| SEQ ID NO: 18 | Amino acid sequence of TPS4 from C. forskohlii; |
| | GenBank accession number KF444509 |
| SEQ ID NO: 19 | Amino acid sequence of CYP76AH16 |
| | from C. forskohlii |
| SEQ ID NO: 20 | Amino acid sequence of CYP76AH8 |
| | from C. forskohlii |
| SEQ ID NO: 21 | Amino acid sequence of CYP76AH11 |
| | from C. forskohlii |
| SEQ ID NO: 22 | Amino acid sequence of CYP76AH15 |
| | from C. forskohlii |
| SEQ ID NO: 23 | Amino acid sequence of CYP76AH17 |
| | from C. forskohlii |
| SEQ ID NO: 24 | Amino acid sequence of ACT1-3B |
| SEQ ID NO: 25 | cDNA encoding ACT1-3B |
| SEQ ID NO: 26 | Amino acid sequence of ACT1-8 |
| | from C. forskohlii |
| SEQ ID NO: 27 | cDNA encoding ACT1-8 from C. forskohlii |
| SEQ ID NO: 28 | DNA Sequence encoding ACT1-8 from C. forskohlii |
| | codon optimized for expression in yeast |
| SEQ ID NO: 29 | cDNA encoding DXS from C. forskohlii |
| SEQ ID NO: 30 | Amino acid sequence of DXS from C. forskohlii |
| SEQ ID NO: 31 | cDNA encoding GGPPS from C. forskohlii |
| SEQ ID NO: 32 | Amino acid sequence of GGPPS from C. forskohlii |
| SEQ ID NO: 33 | cDNA encoding POR from C. forskohlii |
| SEQ ID NO: 34 | Amino acid sequence of POR from C. forskohlii |
| SEQ ID NO: 35 | DNA Sequence encoding TPS2 from C. forskohlii |
| | codon optimized for expression in yeast |
| SEQ ID NO: 36 | DNA Sequence encoding TPS3 from C. forskohlii |
| | codon optimized for expression in yeast |
| SEQ ID NO: 37 | Amion acid sequence of GGPPS from Synechococcus |
| SEQ ID NO: 38 | DNA Sequence encoding GGPPS from Synechococcus |
| | codon optimized for expression in yeast |

The invention will be further described in the following examples, which do not limit the scope of the invention described in the claims.

### EXAMPLES

The Examples that follow are illustrative of specific embodiments of the invention, and various uses thereof. They are set forth for explanatory purposes only, and are not to be taken as limiting the invention.

### Example 1

### Biosynthesis of 13R-MO Derivatives in S. cerevisiae Strain Comprising ACT1-6

A transcriptome prepared from a C. forskohlii cell was used to identify genes encoding the enzymes involved in acetylated and/or oxidized 13R-MO. C. forskohlii root cork total RNA was extracted as described in Pateraki et al., 2014, Plant Physiology 164(3):1222-36. RNA was prepared for sequencing using Illumine TruSeq sample preparation kit v2 (Illumina San Diego, USA), using poly-A selection. The fragments were clustered on cBot and sequenced with paired ends (2×100 bp) on a HiSeq 2500 (IIlumina San Diego, USA), according to the manufacturer's instructions. A total 106.2 million read-pairs were generated. Adaptor sequences were removed from raw reads and reads were trimmed at the ends to phred score 20, using the fastq-mcf tool from ea-utils (https://code.google.com/p/ea-utils/). Processed reads were assembled using Trinity (r2013-02-16) resulting in a total of 263,652 assembled putative transcripts. Transcript abundance estimation was performed using RSEM and the scripts provided with Trinity. Likewise, the putative coding sequences were predicted using the TransDecoder scripts from Trinity.

Mining of the C. forskohlii transcriptome database was performed as described in Zerbe et al., 2013, Plant Physiology 162(2):1073-91, using IBLASTx software acetyltransferase sequences as query. The identified contigs were amplified from single stranded cDNA generated from root cork total RNA using the “SuperScript III First-Strand Synthesis System for RT-PCR” (Invitrogen) and oligo-dT primer. Cloning of the putative ACT cDNAs was achieved after PCR amplification using gene specific primers that were designed based on the in silico sequences of the identified ACT contigs. PCR products were cloned into the pJET1.2 vector and verified by sequencing. For the identified non-full-length cDNA of ACT1-8, full-length transcripts were cloned following 5′ RACE experiments.

Candidate acetyltransferases were tested in a yeast expression system. The genes were controlled by endogenous constitutively active regulatory elements (promoters). The following acetyltransferases were first individually integrated into the strain using standard yeast transformation methods followed by genomic integration: codon-optimized nucleotide sequence encoding C. forskohlii ACT1-6 (SEQ ID NO:11, SEQ ID NO:6); codon-optimized nucleotide sequence encoding C. forskohlii ACT1-7 (SEQ ID NO:12, SEQ ID NO:7); codon-optimized nucleotide sequence encoding ACT1-3A (SEQ ID NO:13, SEQ ID NO:8); nucleotide sequence encoding ACT1-3B (SEQ ID NO:25, SEQ ID NO:24), codon-optimized nucleotide sequence encoding ACT1-4 (SEQ ID NO:14, SEQ ID NO:9); codon-optimized nucleotide sequence encoding C. forskohlii ACT1-1 (SEQ ID NO:15, SEQ ID NO:10). The strain further comprised TPS2 (SEQ ID NO:35, SEQ ID NO:16), TPS3 (SEQ ID NO:36, SEQ ID NO:17), CYP76AH16 (SEQ ID NO:19), CYP76AH8 (SEQ ID NO:20), and CYPAH11 (SEQ ID NO:21).

Selection of transformed yeast cells was performed through the selection marker introduced with the transgenes and by genotyping (using PCR techniques). The selected yeast strains expressing above described genes were cultivated in Synthetic Complete URA dropout medium (SC-URA) at 28° C. for 72 h.

Extraction of acetylated and/or oxidized diterpenes from the yeast culture was performed with ethanol: one volume of yeast culture (cells together with medium) was mixed with one volume of ethanol and heated at 80° C. for 15 min. Acetylated and/or oxidized diterpene products were extracted from the ethanol-yeast culture mixture by one volume of hexane. Acetylated and/or oxidized diterpene metabolites were analyzed by LC-MS.

Results of the LC-MS analysis of yeast cells expressing TPS2 (SEQ ID NO:35, SEQ ID NO:16), TPS3 (SEQ ID NO:36, SEQ ID NO:17), CYP76AH16 (SEQ ID NO:19), CYP76AH8 (SEQ ID NO:20), CYP76AH11 (SEQ ID NO:21) and ACT1-6 (SEQ ID NO:11, SEQ ID NO:6) are shown in FIG. 4. In particular, FIG. 4 shows sodium adducts of ions having an m/z of 433, wherein the molecular weight of forskolin (410 dalton) and the molecular weight of sodium (23 dalton) equals an m/z of 433. The cells expressing ACT1-6 cells produced forskolin, as indicated in FIG. 4.

FIG. 5 shows differences in the products produced by i) yeast cells comprising CYP76AH16 (SEQ ID NO:19), CYP76AH8 (SEQ ID NO:20), and CYP76AH11 (SEQ ID NO:21) (solid lines) and ii) yeast cells comprising CYP76AH16 (SEQ ID NO:19), CYP76AH8 (SEQ ID NO:20), CYP76AH11 (SEQ ID NO:21), and ACT1-6 (SEQ ID NO:11, SEQ ID NO:6) (dotted lines). The peaks labeled (A) represent oxidized products, i.e. products having one or more —OH or ═O groups, and the peaks labeled (B) represent acetylated products, i.e. products having one or more acetyl groups. As shown in FIG. 5, yeast cells comprising CYP76AH16 (SEQ ID NO:19), CYP76AH8 (SEQ ID NO:20), CYP76AH11 (SEQ ID NO:21), and ACT1-6 (SEQ ID NO:11, SEQ ID NO:6) produced acetylated products of oxidized 13R-MO.

### Example 2

### Biosynthesis of 13R-MO Derivatives in S. cerevisiae Strain Comprising ACT1 -8

An S. cerevisiae strain comprising TPS2 (SEQ ID NO:35, SEQ ID NO:16), TPS3 (SEQ ID NO:36, SEQ ID NO:17), CYPAH16 (SEQ ID NO:19), CYPAH8 (SEQ ID NO:20), CYP76AH11 (SEQ ID NO:21), and ACT1-8 (SEQ ID NO:28, SEQ ID NO:26) was also analyzed for forskolin production. A control strain, which did not comprise ACT1-8 (SEQ ID NO:28, SEQ ID NO:26), was also prepared. The selected yeast strains were cultivated in SC-URA at 30° C. for 24 h and then transferred to synthetic complex Feed In Time (FIT) media (M2Plabs) for an additional 72 h at 30° C.

Extraction of acetylated and/or oxidized diterpenes from the yeast culture was performed as described in Example 1, Extracts were spun at 15000×g for 5 min, and the supernatant was subsequently filtered in a 96-well filter plate (0.4 μm). The filtered extract comprising acetylated and/or oxidized diterpene metabolites was subsequently analyzed by LC-MS. The LC-MS system used for analysis was comprised of an Agilent G1312A SL binary pump, Agilent G1367B WP autosampler, Agilent G1316B column oven, Agilent G1315C Starlight DAD detector and a Bruker HCT-Ultra ion trap mass spectrometer using Electron Spray Ionization (ESI). Samples were separated on a Synergi 2.5 μm Fusion-RP C18 column (50×3.2 mm i.d., Phenomenex Inc., Torrance, Calif., USA) at a flow rate of 0.2 mL min⁻¹ with a column temperature held at 25° C. The mobile phase consisted of water with 0.1% formic acid (v/v; solvent A), 50 μM NaCl and 80% acetonitrile with 0.1% formic acid (v/v; solvent B). The gradient program was 30% to 65% B over 24 min followed by a gradient from 65% to 98% over 4 min and 98% B for 1.4 min, followed by a return to starting conditions over 0.1 min, which was then held for 1 min to allow the column to re-equilibrate. Mass spectra were acquired in positive ion mode using a drying temperature of 200° C., a nebulizer pressure of 3.0 bar, and a drying gas flow of 7 L/min.

As shown in FIG. 6A, the strain comprising ACT1-8 (SEQ ID NO:28, SEQ ID NO:26) produced forskolin (solid black line), whereas the control strain did not (solid gray line). Additionally, only minute amounts of deacetylforskolin were present extract of this strain; thus, ACT1-8 has a very strong activity towards deacetylforskolin. A total ion trace is shown in FIG. 6B for the strain comprising ACT1-8, which demonstrates that the main peak was forskolin.

### Example 3

### Biosynthesis of 13R-MO Derivatives in Agrobacterium/Nicotiana benthamiana Heterologous Expression System

Acetyltransferases were tested in a transient Agrobacterium/Nicotiana benthamiana heterologous expression system, which produced oxidized 13R-MO. The following genes were introduced into Nicotiana benthamiana: nucleotide encoding TPS2 polypeptide (SEQ ID NO:16), nucleotide encoding TPS3 polypeptide (SEQ ID NO:17), nucleotide encoding CYP76AH16 polypeptide (SEQ ID NO:19), nucleotide encoding CYP76AH15 polypeptide (SEQ ID NO:22), and nucleotide encoding CYP76AH11 (SEQ ID NO:21).

The following sequences were also individually introduced into Nicotiana benthamiana: ACT1-6 (SEQ ID NO:1, SEQ ID NO:6), ACT1-7 (SEQ ID NO:2, SEQ ID NO:7), ACT1-3A (SEQ ID NO:3, SEQ ID NO:8), ACT1-3B (SEQ ID NO:25, SEQ ID NO:24), ACT1-1 (SEQ ID NO:5, SEQ ID NO:10), and ACT1-8 (SEQ ID NO:27, SEQ ID NO:26).

For infiltration, 20 mL of agrobacteria cultures for each individual biosynthetic gene was grown overnight. The agrobacteria were harvested by centrifugation at 4000×g for 10 min and resuspended in 50 mL water. The OD₆₀₀ of the independent samples/cultures were normalized and adjusted with water to a final concentration of OD₆₀₀ of 1 before combining them for agroinfiltration in tobacco leaves. For every individual tobacco plant, at least 3 expanded leaves were agroinfiltrated using the same cultures combination. Each leaf served as an experimental replicate.

Extraction of acetylated and/or oxidized diterpenes from leaves of the Nicotiana benthamiana plants was performed with 80% methanol. Acetylated and/or oxidized diterpene metabolites were analyzed by LC-MS, and the results are shown in FIG. 7A and FIG. 7B. Plants expressing ACT1-6 (SEQ ID NO:1, SEQ ID NO:6), ACT1-3A (SEQ ID NO:3, SEQ ID NO:8), ACT1-3B (SEQ ID NO:25, SEQ ID NO:24), and ACT1-1 (SEQ ID NO:5, SEQ ID NO:10) each produced forskolin (FIG. 7A). As shown in FIG. 78, co-expression of C. forskohlii DXS (SEQ ID NO:29, SEQ ID NO:30), C. forskohlii GGPPS (SEQ ID NO:31, SEQ ID NO:32), TPS2 (SEQ ID NO:16), and TPS3 (SEQ ID NO:17) in N. benthamiana does not result in accumulation of deacetylforskolin or forskolin. Co-expression of C. forskohiii DXS (SEQ ID NO:29, SEQ ID NO:30), C. forskohlii GGPPS (SEQ ID NO:31, SEQ ID NO:32), TPS2 (SEQ ID NO:16), TPS3 (SEQ ID NO:17), CYP76AH15 (SEQ ID NO:22), CYP76AH11 (SEQ ID NO:21), and CYPAH16 (SEQ ID NO:19) in N. benthamiana resulted in accumulation of deacetylforskolin. Co-expression of C. forskohlii DXS, C. forskohlii GGPPS, TPS2, TPS3, CYP76AH15, CYP76AH11, CYPAH16, and either ACT1-6 or ACT1-8 in N. benthamiana resulted in accumulation of forskolin.

FIG. 7C shows LC-qTOF-MS analysis of 13R-MO derived diterpenoids obtained by transient expression of combinations of C. forskohlii CYP and ACT encoding genes in N. benthamiana. Total ion chromatograms (TIC) from extracts expressing CYP76AH8 (SEQ ID NO:20), CYP76AH11 (SEQ ID NO:21), CYP76AH16 (SEQ ID NO:19), and ACT1-6 (SEQ ID NO:1, SEQ ID NO:6) or expressing CYP76AH8 (SEQ ID NO:20), CYP76AH11 (SEQ ID NO:21), CYP76AH16 (SEQ ID NO:19), and ACT1-8 (SEQ ID NO:27, SEQ ID NO:26) are shown. Oxidized and acetylated 13R-MO derived diterpenoids are marked with gray bars.

### Example 4

### Engineering of Forskolin-Producing S. cerevisiae Strain

C. forskohlii POR (SEQ ID NO:33, SEQ ID NO:34), Synechococcus GGPPS (SEQ ID NO:38, SEQ ID NO:37), TPS2 (SEQ ID NO:35, SEQ ID NO:16), TPS3 (SEQ ID NO:36, SEQ ID NO:17), CYP76AH15 (SEQ ID NO:22), CYP76AH11 (SEQ ID NO:21), CYP76AH16 (SEQ ID NO:19), and ACT1-8 (SEQ ID NO:28, SEQ ID NO:26) were stably integrated into the genome of an S. cerevisiae strain engineered to produce high amounts of 13R-MO. C. forskohlii POR encodes for NADPH-dependent cytochrome P450 oxidoreductase required to support the P450s activity. All genes were cloned into yeast genome integration plasmids by the USER technique targeting incorporation into site XI-2. See Nour-Eldin et al., 2010, Plant Secondary Metabolism Engineering 643:185-200 and Mikkelsen et al., 2012, Metabolic Engineering 14:104-11. Transformants were verified by PCR on genomic DNA for correct insertion of heterologous genes and grown and tested in 96 deepwell plates. The yeast strain was cultivated for 140 h in a 5 L fermentor using minimal medium and glucose limited conditions. Forskolin production was monitored using withdrawn culture aliquots. Forskolin was extracted from the mixture of yeast cells and culture broth using 85% ethanol and incubation for 20 min at 75° C. and the extract centrifuged (10000×g for 5 min) to precipitate yeast debris. The supernatant obtained was used directly for LC-MS analysis and forskolin quantification.

For forskolin quantification, aliquots of yeast (with broth) were combined with methanol to give a concentration of 85% methanol, incubated at 75° C. for 20 min, filtered and then analyzed by LC-MS. Quantification was based on a standard calibration curve of forskolin purchased from Sigma-Aldrich. An Ultimate 3000 UHPLC⁺ Focused system (Dionex Corporation, Sunnyvale, Calif., USA) coupled to a Bruker Compact ESI-QTOF-MS (Bruker Daltonik, Bremen, Germany) was used to quantify forskolin. Samples were separated on a Kinetex XB-C18 column (100×2.1 mm i.d., 1.7 μm particle size, 100 Å pore size; Phenomenex Inc., Torrance, Calif., USA) maintained at 40° C. with a flow rate of 0.3 mL/min and mobile phase consisting of 0.05% (v/v) formic acid in water (solvent A) and 0.05% (v/v) formic acid in acetonitrile (solvent B). The gradient LC method was as follows: solvent B was held at 20% for 30 s, then ramped to 100% over 8.5 min, held at 100% for 2 min, decreased to 20% over 30 s and held for 3.5 min to give an overall run time of 15 min. The ESI source parameters were as follows: capillary voltage, 4500 V; nebulizer pressure 1.2 bar; dry gas flow, 8 L/min; dry gas temperature, 250° C. The QTOF-MS was operated in MS only mode with collision cell energy of 7 eV and collision cell RF of 500 Voₗ), Ions were monitored in the positive mode over a range of 50-1300 m/z and spectra collected at a rate of 2 Hz. As shown in FIG. 8, forskolin levels accumulated to over 40 mg/L yeast culture for the strain comprising CYP76AH15 (SEQ ID NO:22), CYP76AH11 (SEQ ID NO:21), CYP76AH16 (SEQ ID NO:19), and ACT1-8 (SEQ ID NO:28, SEQ ID NO:26).

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

TABLE 2
Sequences disclosed herein.

SEQ ID NO: 1

| | |
|---|---|
| atgaaggtgg aaagatttag caggaaactc ataaaaccgc acaccccaac ccccgaaaat | 60 |
| ctgaagaagt ataagctttc tcttttagac aaatgcttgg ggcacgataa ttttgctatt | 120 |
| gttctgtttt acgaatcgaa accaagaaac aagagtgagt tggaagaatc actggaaaaa | 180 |
| gtcctggtgg atttctaccc tcttgctgga agacacacca tgaatgatca catagttgat | 240 |
| tgcagtgatg tgggcgccgt gtttgtcgaa gcagaagctc tagacgtcga gctgacgatg | 300 |
| gatgagctcg ttaagaacat ggaggcccag actatccatc atctccttcc caatcaatat | 360 |
| ttctcagctg atgctccaaa tccactcttg tcaatccagg tgacacattt cccatctggt | 420 |
| ggcctagcaa ttggcatcgc cgtttctcac gctgtcttcg atggtttttc gctgggggtg | 480 |
| ttcgtcgccg cctggtcgaa ggccaccatg aacccagatc ggaagatcaa aatcacaccg | 540 |
| tctttcgatc ttccctcgtt gcttccttac aaggatgaca attttggatt aactgccgcc | 600 |
| gaaattgtca gccagagtga agatattgtt gttaagaggt ttattttcgg gaaggaggca | 660 |
| ataacgaggt tgagatcaaa gctaagccca aatcgcaatg ggaaaaagat ctctcgtgtt | 720 |
| cgagtggtgt gtgccgttat agtgaaagcc ttgatgggcc tggaacgcgc caaacatggc | 780 |
| aaaacaagag atttcttgat cactcaatcg attaacatgc gcgagagaac aaaagcacct | 840 |
| ctacagaagc atgcttgtgg gaatctggca gtcttgtcgt gcacgcgacg tgtagaagcc | 900 |
| gaggagatga tggagctgca gaacttggtt aatctgatcg gagattctac agagaaggac | 960 |
| atagctgatt ttgctgaatt gctatctcct gatcaagttg ggcgtgatat catcatcaag | 1020 |
| atgatgaagt ctttcatgca attcttggat aatgacattt attctgtatg cttcactgat | 1080 |
| tggagtaagt ttgaatttta cgaagctgat tttgggttcg ggaagcctgt ttggatggcc | 1140 |
| gctggaccgc aacgcccaat cattagcacc gctattctca tgagcgacag agagggtgat | 1200 |
| ggaattgagg catggctgca tctcaacaaa aatgacatgc ttatcttcga gcaagatgaa | 1260 |
| gaaatcaaat tatttactac ctaa | 1284 |

SEQ ID NO: 2

| | |
|---|---|
| atgaaggtgg aaagatttag caggaaactc ataaaaccgc acaccccaac ccccgaaaat | 60 |
| ctcaagaatt ataagctttc tcttttagat aaatgcttgg tgcaggataa ttttgctgtt | 120 |
| gtgctgtttt acgaatcgaa accaagaaac aagagtgagt tggaagaatc actagaaaaa | 180 |
| gtccttgtcg atttctaccc tcttgctgga agatacacca tgaatgatca catagttgat | 240 |
| tgcagtgatg agggcgccgt gtttgtcgaa gctgaagctc tggatgctga gctgacgatg | 300 |
| gatcagctcg tcaagaatat ggaggcccag actatccatc atctccttcc ggatcaatat | 360 |
| ttcccagctg atgctccaat tccactcctc tcaatccagg tcacacattt cccatttggg | 420 |
| ggcctggcaa ttgccatcgt cgtttctcac gctgtattcg aaggtttttc actcggggtg | 480 |
| ttcgtcgccg cctggtcgaa ggccaccatt aatccagatg tgaagatcga aatcaccccg | 540 |
| tctttcgatc ttccctcatt gcttccatac aaggatgacg atttcggatt aactgactgt | 600 |
| gaaattatta acctgtgtga ggatattgtt gttaagaggt ttatgtttgg gaaggaggct | 660 |
| ataacgaggt tgagatcaag actaagccca aatcacaatg ggaaaacgat ctctcgtgtt | 720 |
| cgagtggtgt gtgccgttat agtgaaagcc ttgatgggcc tggaacgcgc caaacatggc | 780 |
| aaaacaagag atttcttgat cactcaatcg attaacatgc gcgagagaac aaaagcacct | 840 |
| ctacagaagc atgcttgtgg gaatctggca gtcttgtcgt gcacgcgacg tgtagaagcc | 900 |
| gaggagatga tggagctgca gaacttggtt aatctgatcg gagaatctac tgagaaggac | 960 |
| atagctcatt attctgaatt gctgtctcat aatcaatttg ggcgtgatat catcgtcaac | 1020 |
| gtgatgaaat ctctcatgca attcttggat cctgacattt attctgtatg cttcactgat | 1080 |
| tggagtaagt ttagattcta cgaagctgat tttgggttcg ggaagcctgt ttggacggcc | 1140 |
| gttggaccgc aacgcccaat cattaccacc gctattctca tgaacaacag agagggtgat | 1200 |
| ggaattgagg catggctgca tctcaacaaa aatgacatgc ttatcttcga gcaagatgaa | 1260 |
| gaaatcaaat tatttactac ctaa | 1284 |

SEQ ID NO: 3

| | |
|---|---|
| atgaaggtgg aaagatttag caggaaattc ataaaaccac acaccccgac ccccgaaaat | 60 |
| ctgaagaagt ataagctttc tcttttagac aaatgcttgg ggcacgataa ttttgctatt | 120 |
| gttctgtttt acgaatcgaa accaagaaac aagagtgagt tggaagaatc actggaaaaa | 180 |
| gtcctggtgg atttctaccc tcttgctgga agacacacca tgaatgatca catagttgat | 240 |
| tgcagtgatg tgggcgccgt gtttgtcgaa gcagaagctc tagacgtcga gctgacgatg | 300 |
| gatgagctcg ttaagaacat ggaggcccag actatccatc atctccttcc caatcaatat | 360 |
| ttctcagctg atgctccaaa tccactcttg tcaatccagg tgacacattt cccatctggt | 420 |
| ggcctagcaa ttggcatcgc cgtttctcac gctgtcttcg atggtttttc gctgggggtg | 480 |
| ttcgtcgccg cctggtcgaa ggccaccatg aacccagatc ggaagatcaa aatcacaccg | 540 |
| tctttcgatc ttccctcgtt gcttccttac aaggatgaca attttggatt aactgccgcc | 600 |
| gaaattgtca gccagagtga agatattgtt gttaagaggt ttattttcgg gaaggaggca | 660 |
| ataacgaggt tgagatcaaa gctaagccca aatcgcaatg ggaaaaagat ctctcgtgtt | 720 |
| cgagtggtgt gtgccgttat agtgaaagcc ttgatgggcc tggaacgcgc caaaacaaga | 780 |
| gatttcatga tctgtcaagg gatcaacatg cgtgagagaa caaaggcacc tctacagaag | 840 |
| catgcttgtg ggaatctggc ggtctcgtct tacacgcgac gtgtagccgc agcagaagcc | 900 |
| gaggagctgc agagtttggt aaatctaata ggtgattcta ttgagaagag catagctgat | 960 |
| tatgctgata ttctttcttc tgatcaagat gggcgtcata tcatcagcac gatgatgaaa | 1020 |
| tctttcatgc aattcgcggc tcctgacata aaagctatat ccttcactga ttggagtaag | 1080 |
| tttggattct accaagttga ttttgggttc gggaagcctg tttggacggg tgttcggccc | 1140 |
| gaacgcccaa tctttagcgc cgctattctc atgagcaaca gagagggtga tggaattgag | 1200 |
| gcatggcttc atcttgacaa aaatgacatg cttatcttcg agcaagatga agaaatcaaa | 1260 |
| ttattaatta ctacctaa | 1278 |

SEQ ID NO: 4

| | |
|---|---|
| atgaaggtgg aaagatttag caggaaattc ataaaaccac acaccccgac ccccgaaaat | 60 |
| ctgaagaagt ataagctttc tettttagat aaatgcttgg ggcacgataa ttttgctatt | 120 |
| gttctgtttt acgaatcgaa accaagaaac aagagtgagt tggaagaatc actagaaaaa | 180 |
| gtccttgtcg atttctaccc tcttgctgga agatacacca tgaatgatca catagttgat | 240 |
| tgcagtgatg agggcgccgt gtttgtcgaa gccgaagctc caaacgtcga gctgacggtg | 300 |
| gatcaactcg tcaagaacat ggaggcccag actatccatg atttccttcc cgatcaatat | 360 |
| ttcccagctg atgctccaaa tccactcctc tcgatccagg tcacgcattt cccatgtggt | 420 |
| ggcttggcga ttggcatcgt tgtttctcac gctgtcttcg atggtttttc gctgggggtg | 480 |
| ttccttgccg cctggtcgaa ggccaccatg aacccagaga ggaagatcga aatcaccccg | 540 |
| tctttcgatc ttccttcgtt gcttccatac aaggatgaaa gtttcggatt aaatttcagc | 600 |
| gaaattgtca aggctgaaaa tattgttgtg aagaggctta atttcgggaa ggaggctata | 660 |
| acgaggttga gatcaagact aagcccaaat cacaatggga aaacgatctc tcgtgttcga | 720 |
| gttgtgtgtg cccttatagt gaaagccttg atgggcctgg aactcgccaa gcatggcaaa | 780 |
| acaagagatt tcatgatctc tcaagggatt aacatgcgcg agagaacaaa agcacctcta | 840 |
| cacaagcatg cttgtgggaa tctagcaatc ttgtcgtgca cgcgacgtgt agaagccgag | 900 |
| gagatgatgg acctgcaaaa cttggttaat ctgatcggag aatctactga gaaggacata | 960 |
| gctcattatt ctgaattgct gtctcataat caatttgggc gtgatatcat cgtcaacgtg | 1020 |
| atgaaatctc tcatgcaatt cttggatcct gacatttatt ctgtatgctt cactgattgg | 1080 |
| agtaagttta gattctacga agctgatttt gggttcggga agcctgtttg gacggccgtt | 1140 |
| ggaccgcaac gcccaatcat taccaccgct attctcatga acaacagaga gggtgatgga | 1200 |
| attgaggcat ggctgcatct caacaaaaat gacatgctta tcttcgagca agatgaagaa | 1260 |
| atcaaattat ttactaccta a | 1281 |

SEQ ID NO: 5

| | |
|---|---|
| atgaaggtgg aaagatttag caggaaatta ataaaaccag tcaccccaac tccacaaaac | 60 |
| ctcaagaact tcaacctttc gattttagat aaatgtcttc cgccgattaa atttggtgtt | 120 |
| gttttgtttt atgaatctaa accaggaaat aagagcgagt tggaagaatc actaaaagaa | 180 |
| gttctggtcg acttctaccc tcttgctgga agacacacca tcaatgatcc cgtggttgat | 240 |
| tgcagtgatc agggcgccgt gttcgtcgaa gccgaagctc tagacaccga gctgaccatg | 300 |
| gatcagctgg tgttgaataa gatggagatc cagaaagtcg atcaattcct tcccgatgaa | 360 |
| tgcatccaag ctgatgctcc gaatccactc ctgtggatcc aggtgacaca tttcccatcg | 420 |
| ggtgggctgg cgatcggcgt tgcggtttct cactctgtct tcgattcctt ctcgctgggg | 480 |
| gtgttcatcg ccgcctggtc caaggccaca atgaatccag gtaggatgat cgaaatcacc | 540 |
| ccgtctttcg atgttccctc gttgcttcca tgcaaggatc acgatttcga gatagctctc | 600 |
| aatgaaatta cggatcaggg tgaaagcttc gttgttaaga ggctcgtgtt cggtaaggag | 660 |
| gctataacga ggttgagatc aaaactaagc ctaaatcaag atggtaaaac gatctctcgt | 720 |
| gttcgtgttg tgggtgccgt tctagtgaaa gccctgatcg gcctggaatg tggcaaacac | 780 |
| ggcagaagaa aagatctcgt gatctctctg ccggttaaca tgcgtgagag aacaaacaca | 840 |
| cctctacaga atccaaagca tgcttgcggg aatctggcgg tcatttcgct cacgcgatgc | 900 |
| gtagctgcag cagaagctga ggagatgggg ctgcaggagt tggtaaatct acttggagat | 960 |
| gcgattggaa aagcgatagc tgatcatgct gaaatgttgt ctcctaatca agaagggtgt | 1020 |
| gacatcatta ttaatgattt caagaacttt ttaacattat tcgggactcc taacacaaat | 1080 |
| attattactc ttactgattg gagtaagttt ggattctacg aagctgattt tgggtttggg | 1140 |
| aagcctgttt ggaccagcag cggacagcaa tccctgagcg ttaccaccat tgtgctcatg | 1200 |
| aacaacaaag agggcgatgg aatcgaggca tggctgcatc tcaacaaaaa tgacatgctt | 1260 |
| ttcttcgagc aagatgaaga aatcaaatta tttactacct aa | 1302 |

SEQ ID NO: 6

| | |
|---|---|
| MKVERFSRKL IKPHTPTPEN LKKYKLSLLD KCLGHDNFAI VLFYESKPRN KSELEESLEK | 60 |
| VLVDFYPLAG RHTMNDHIVD CSDVGAVFVE AEALDVELTM DELVKNMEAQ TIHHLLPNQY | 120 |
| FSADAPNPLL SIQVTHFPSG GLAIGIAVSH AVFDGFSLGV FVAAWSKATM NPDRKIKITP | 180 |
| SFDLPSLLPY KDDNFGLTAA EIVSQSEDIV VKRFIFGKEA ITRLESKLSP NRNGKKISRV | 240 |
| RVVCAVIVKA LMGLERAKHG KTRDFLITQS INMRERTKAP LQKHACGNLA VLSCTRRVEA | 300 |
| EEMMELQNLV NLIGDSTEKD IADFAELLSP DQVGRDIIIK MMKSFMQFLD NDIYSVCFTD | 360 |
| WSKFEFYEAD FGFGKPVWMA AGPQRPIIST AILMSDREGD GIEAWLHLNK NDMLIFEQDE | 420 |
| EIKLFTT | 427 |

SEQ ID NO: 7

| | |
|---|---|
| MKVERFSRKL IKPHTPTPEN LKNYKLSLLD KCLVQDNFAV VLFYESKPRN KSELEESLEK | 60 |
| VLVDFYPLAG RYTMNDHIVD CSDEGAVFVE AEALDAELTM DQLVKNMEAQ TIMILLPDQY | 120 |
| FPADAPIPLL SIQVTHFPFG GLAIAIVVSH AVFEGFSLGV FVAAWSKATI NPDVKIEITP | 180 |
| SFDLPSLLPY KDDDFGLTDC EIINLCEDIV VKRFMFGKEA ITRLRSRLSP NHNGKTISRV | 240 |
| RVVCAVIVKA LMGLERAKHG KTRDFLITQS INMRERTKAP LQKHACGNLA VLSCTRRVEA | 300 |
| EEMMELQNLV NLIGESTEKD IAHYSELLSH NQFGRDIIVN VMKSLMQFLD PDIYSVCFTD | 360 |
| WSKFRFYEAD FGFGKPVWTA VGPQRPIITT AILMNNREGD GIEAWLHLNK NDMLIFEQDE | 420 |
| EIKLFTT | 427 |

SEQ ID NO: 8

| | |
|---|---|
| MKVERFSRKF IKPHTPTPEN LKKYKLSLLD KCLGHDNFAI VLFYESKPRN KSELEESLEK | 60 |
| VLVDFYPLAG RHTMNDHIVD CSDVGAVFVE AEALDVELTM DELVKNMEAQ TIHHLLPNQY | 120 |
| FSADAPNPLL SIQVTHFPSG GLAIGIAVSH AVFDGFSLGV FVAAWSKATM NPDRKIKITP | 180 |
| SFDLPSLLPY KDDNFGLTAA EIVSOSEDIV VKRFIFGKEA ITRLRSKLSP NRNGKKISRV | 240 |
| RVVCAVIVKA LMGLERAKTR DFMICQGINM RERTKAPLQK HACGNLAVSS YTRRVAAAEA | 300 |
| EELQSLVNLI GDSIEKSIAD YADILSSDQD GRHIISTMMK SFMQFAAPDI KAISFTDWSK | 360 |
| FGFYQVDFGF GKPVWTGVRP ERPIFSAAIL MSNREGDGIE AWLHLDKNDM LIFEQDEEIK | 420 |
| LLITT | 425 |

SEQ ID NO: 9

| | |
|---|---|
| MKVERFSRKF IKPHTPTPEN LKKYKLSLLD KCLGHDNFAI VLFYESKPRN KSELEESLEK | 60 |
| VLVDFYPLAG RYTMNDHIVD CSDEGAVFVE AEAPNVELTV DQLVKNMEAQ TIHDFLPDQY | 120 |
| FPADAPNPLL SIQVTHFPCG GLAIGIVVSH AVFDGFSLGV FLAAWSKATM NPERKIEITP | 180 |
| SFDLPSLLPY KDESFGLNFS EIVKAENIVV KRLNFGKEAI TRLRSRLSPN HNGKTISRVR | 240 |
| VVCALIVKAL MGLELAKHGK TRDFMISQGI NMRERTKAPL HKHACGNLAI LSCTRRVEAE | 300 |
| EMMDLONLVN LIGESTEKDI AHYSELLSHN QFGRDIIVNV MKSLMQFLDP DIYSVCFTDW | 360 |
| SKFRFYEADF GFGKPVWTAV GPQRPIITTA ILMNNREGDG IEAWLHLNKN DMLIFEQDEE | 420 |
| IKLFTT | 426 |

SEQ ID NO: 10

| | |
|---|---|
| MKVERFSRKL IKPVTPTPQN LKNFNLSILD KCLPPIKFGV VLFYESKPGN KSELEESLKE | 60 |
| VLVDFYPLAG RHTINDPVVD CSDQGAVFVE AEALDTELTM DQLVLNKMEI QKVDQFLPDE | 120 |
| CIQADAPNPL LWIQVTHFPS GGLAIGVAVS HSVFDSFSLG VFIAAWSKAT MNPGRMIEIT | 180 |
| PSFDVPSLLP CKDHDFEIAL NEITDQGESF VVKRLVFGKE AITRLRSKLS LNQDGKTISR | 240 |
| VRVVGAVLVK ALIGLECGKH GRRKALVISL PVNMRERTNT PLQNPKHACG NLAVISLTRC | 300 |
| VAAAEAEEMG LQELVNLLGD AIGKAIADHA EMLSPNQEGC DIIINDFKNF LTLFGTPNTN | 360 |
| IITLTDWSKF GFYEADFGFG KPVWTSSGQQ SLSVTTIVLM NNKEGDGIEA WLHLNKNDML | 420 |
| FFEQDEEIKL FTT | 433 |

SEQ ID NO: 11

| | |
|---|---|
| atgaaggtcg aaagattctc cagaaagttg attaagccac atactccaac tccagaaaac | 60 |
| ttgaagaagt acaagttgtc cttgttggat aagtgcttgg gtcatgataa tttcgccatc | 120 |
| gttttgttct acgaatccaa gccaagaaac aagtccgaat tggaagaatc cttggaaaag | 180 |
| gttttggttg acttttatcc attggctggt agatacacca tgaacgatca tatagttgat | 240 |
| tgctctgatg ttggtgccgt ttttgttgaa gctgaagctt tggatgttga attgaccatg | 300 |
| gatgaattgg tcaagaacat ggaagctcaa accatccatc atttgttgcc aaatcaatac | 360 |
| ttctctgctg atgctccaaa tcctttgttg tctattcaag ttacccattt cccatctggt | 420 |
| ggtttggcta ttggtattgc tgtttctcat gctgttttcg acggtttttc tttgggtgtt | 480 |
| ttcgttgctg cttggtctaa agctactatg aatccagata gaaagatcaa gatcacccca | 540 |
| tcttttgact tgccatcttt gttaccatac aaggatgata acttcggttt gactgctgct | 600 |
| gaaatcgttt ctcaatctga agatatcgtc gtcaagagat tcatcttcgg taaagaagct | 660 |
| atcactagat tgagatccaa gttgtctcca aacagaaacg gtaagaagat ctccagagtt | 720 |
| agagttgttt gtgccgttat agttaaggct ttgatgggtt tggaaagagc taaacacggt | 780 |
| aagactagag atttcttgat cacccaatcc atcaacatga gagaaagaac aaaagcccca | 840 |
| ttgcaaaaac atgcttgtgg taatttggct gttttgtctt gtaccagaag agttgaagcc | 900 |
| gaagaaatga tggaattgca aaacttggtt aacttgatcg gtgactctac cgaaaaggat | 960 |
| attgctgatt tcgccgaatt attgtcccca gatcaagttg gtagagacat cattatcaag | 1020 |
| atgatgaagt ccttcatgca attcttggac aacgacatct actctgtttg tttcactgat | 1080 |
| tggtctaagt tcgaattcta cgaagccgat tttggttttg gtaaaccagt ttggatggct | 1140 |
| gctggtccac aaagaccaat tatttctact gccatcttga tgtccgatag agaaggtgat | 1200 |
| ggtattgaag cttggttgca tttgaacaag aacgacatgt tgatcttcga acaagacgaa | 1260 |
| gaaatcaagt tgttcaccac ctga | 1284 |

SEQ ID NO: 12

| | |
|---|---|
| atgaaggtcg aaagattctc cagaaagttg attaagccac atactccaac tccagaaaac | 60 |
| ttgaagaact acaagttgtc cttgttggat aagtgcttgg tccaagataa tttcgccgtt | 120 |
| gttttgttct acgaatccaa gccaagaaac aagtccgaat tggaagaatc cttggaaaag | 180 |
| gttttggttg acttttatcc attggctggt agatacacca tgaacgatca tatagttgat | 240 |
| tgctctgatg aaggtgccgt ttttgttgaa gctgaagctt tggatgctga attgactatg | 300 |
| gatcaattgg tcaagaacat ggaagcccaa accattcatc atttgttgcc agatcaatac | 360 |
| tttccagctg atgctccaat tcctttgttg tctattcaag ttacccattt cccatttggt | 420 |
| ggtttggcta ttgctatcgt tgtttctcat gctgttttcg acggtttttc tttgggtgtt | 480 |
| ttcgttgctg cttggtctaa agctactatt aacccagatg tcaagatcga aattacccca | 540 |
| tcttttgact tgccatcctt gttgccatac aaggacgatg attttggttt gaccgattgc | 600 |
| gaaatcatca acttgtgtga agatatcgtc gtcaagagat tcatgttcgg taaagaagct | 660 |
| atcaccagat tgagatctag attgtctcca aaccataacg gtaagaccat ctctagagtt | 720 |
| agagttgttt gtgccgttat cgttaaggct ttgatgggtt tggaaagagc taaacacggt | 780 |
| aaaaccagag atttcttgat cacccaatcc atcaacatga gagaaagaac aaaagcccca | 840 |
| ttgcaaaaac atgcttgtgg taatttggct gttttgtctt gtaccagaag agttgaagcc | 900 |
| gaagaaatga tggaattgca aaacttggtt aacttgatcg gtgaatccac cgaaaaggat | 960 |
| attgctcact actccgaatt attgtcccac aatcaattcg gtagagacat catcgttaac | 1020 |
| gtcatgaagt ctttgatgca attcttggat ccagacatct actctgtttg tttcactgat | 1080 |
| tggtctaagt tcagattcta cgaagctgat ttcggttttg gtaaaccagt ttggactgct | 1140 |
| gttggtccac aaagaccaat tattactacc gccattttga tgaacaacag agaaggtgat | 1200 |
| ggtattgaag cttggttgca tttgaacaag aacgacatgt tgatcttcga acaagacgaa | 1260 |
| gaaatcaagt tgttcaccac ctga | 1284 |

SEQ ID NO: 13

| | |
|---|---|
| atgaaggtcg aaagattctc cagaaagttc attaagccac atactccaac tccagaaaac | 60 |
| ttgaagaagt acaagttgtc cttgttggat aagtgcttgg gtcatgataa tttcgccatc | 120 |
| gttttgttct acgaatccaa gccaagaaac aagtccgaat tggaagaatc cttggaaaag | 180 |
| gttttggttg acttttatcc attggctggt agatacacca tgaacgatca tatagttgat | 240 |
| tgctctgatg ttggtgccgt ttttgttgaa gctgaagctt tggatgttga attgaccatg | 300 |
| gatgaattgg tcaagaacat ggaagctcaa accatccatc atttgttgcc aaatcaatac | 360 |
| ttctctgctg atgctccaaa tcctttgttg tctattcaag ttacccattt cccatctggt | 420 |
| ggtttggcta ttggtattgc tgtttctcat gctgttttcg acggtttttc tttgggtgtt | 480 |
| ttcgttgctg cttggtctaa agctactatg aatccagata gaaagatcaa gatcacccca | 540 |
| tcttttgact tgccatcttt gttaccatac aaggatgata acttcggttt gactgctgct | 600 |
| gaaatcgttt ctcaatctga agatatcgtc gtcaagagat tcatcttcgg taaagaagct | 660 |
| atcactagat tgagatccaa gttgtctcca aacagaaacg gtaagaagat ctccagagtt | 720 |
| agagttgttt gtgccgttat agttaaggct ttgatgggtt tggaaagagc taagactaga | 780 |
| gatttcatga tctgccaagg tatcaacatg agagaaagaa caaaagcccc attgcaaaaa | 840 |
| catgcttgtg gtaatttggc cgtttcttca tacactagaa gagttgctgc tgcagaagca | 900 |
| gaagaattgc aatctttggt taacttgatc ggtgactcca tcgaaaagtc tattgctgat | 960 |
| tacgccgata tcttgtcctc tgatcaagat ggtagacata tcatctccac catgatgaag | 1020 |
| tctttcatgc aatttgctgc cccagatatt aaggctattt ctttcactga ttggtctaag | 1080 |
| ttcggtttct accaagttga ttttggtttc ggtaaaccag tttggactgg tgttagacca | 1140 |
| gaaagaccaa ttttttccgc tgccattttg atgtctaaca gagaaggtga tggtattgaa | 1200 |
| gcttggttgc atttggataa gaacgacatg ttgatcttcg aacaagacga agaaatcaag | 1260 |
| ttgttgatca ccacctga | 1278 |

SEQ ID NO: 14

| | |
|---|---|
| atgaaggtcg aaagattctc cagaaagttc attaagccac atactccaac tccagaaaac | 60 |
| ttgaagaagt acaagttgtc cttgttggat aagtgcttgg gtcatgataa tttcgccatc | 120 |
| gttttgttct acgaatccaa gccaagaaac aagtccgaat tggaagaatc cttggaaaag | 180 |
| gttttggttg acttttatcc attggctggt agatacacca tgaacgatca tatagttgat | 240 |
| tgctctgatg aaggtgccgt ttttgttgaa gctgaagctc caaatgttga attgaccgtt | 300 |
| gatcaattgg tcaagaacat ggaagctcaa accatccatg atttcttgcc agatcaatac | 360 |
| tttccagctg atgctcctaa tcctttgttg tctattcaag ttacccattt cccatgtggt | 420 |
| ggtttggcta ttggtatagt tgtttctcat gctgttttcg acggtttctc tttgggtgtt | 480 |
| tttttggctg cttggtctaa ggctactatg aatccagaaa gaaagatcga aatcacccca | 540 |
| tcttttgact tgccatcttt gttgccttac aaggacgaat cttttggttt gaacttctcc | 600 |
| gaaatcgtta aggccgaaaa catcgttgtt aagagattga acttcggtaa agaagccatc | 660 |
| accagattga gatctagatt gtctccaaac cataacggta agaccatctc tagagttaga | 720 |
| gttgtttgtg ccttgattgt caaggctttg atgggtttgg aattggctaa acatggtaag | 780 |
| actagagact tcatgatctc ccaaggtatc aacatgagag aaagaacaaa agccccattg | 840 |
| cacaaacatg cttgtggtaa tttggccatt ttgtcttgta ccagaagagt tgaagccgaa | 900 |
| gaaatgatgg acttgcaaaa cttggttaac ttgatcggtg aatccaccga aaaggatatt | 960 |
| gctcactact ccgaattatt gtcccacaat caattcggta gagacatcat cgttaacgtt | 1020 |
| atgaagtcct tgatgcaatt cttggatcca gatatctact ctgtttgttt cactgactgg | 1080 |
| tccaagttca gattttacga agctgatttt ggtttcggta agccagtttg gactgctgtt | 1140 |
| ggtccacaaa gaccaattat tactaccgcc attttgatga acaacagaga aggtgatggt | 1200 |
| attgaagctt ggttgcattt gaacaagaac gacatgttga tcttcgaaca agacgaagaa | 1260 |
| atcaagttgt tcaccacctg a | 1281 |

SEQ ID NO: 15

| | |
|---|---|
| atgaaggtgg aaagatttag caggaaatta ataaaaccag tcaccccaac tccacaaaac | 60 |
| ctcaagaact tcaacctttc gattttagat aaatgtcttc cgccgattaa atttggtgtt | 120 |
| gttttgtttt atgaatctaa accaggaaat aagagcgagt tggaagaatc actaaaagaa | 180 |
| gttctggtcg acttctaccc tcttgctgga agacacacca tcaatgatcc cgtggttgat | 240 |
| tgcagtgatc agggcgccgt gttcgtcgaa gccgaagctc tagacaccga gctgaccatg | 300 |
| gatcagctgg tgttgaataa gatggagatc cagaaagtcg atcaattcct tcccgatgaa | 360 |
| tgcatccaag ctgatgctcc gaatccactc ctgtggatcc aggtgacaca tttcccatcg | 420 |
| ggtgggctgg cgatcggcgt tgcggtttct cactctgtct tcgattcctt ctcgctgggg | 480 |
| gtgttcatcg ccgcctggtc caaggccaca atgaatccag gtaggatgat cgaaatcacc | 540 |
| ccgtctttcg atgttccctc gttgcttcca tgcaaggatc acgatttcga gatagctctc | 600 |
| aatgaaatta cggatcaggg tgaaagcttc gttgttaaga ggctcgtgtt cggtaaggag | 660 |
| gctataacga ggttgagatc aaaactaagc ctaaatcaag atggtaaaac gatctctcgt | 720 |
| gttcgtgttg tgggtgccgt tctagtgaaa gccctgatcg gcctggaatg tggcaaacac | 780 |
| ggcagaagaa aagatctcgt gatctctctg ccggttaaca tgcgtgagag aacaaacaca | 840 |
| cctctacaga atccaaagca tgcttgcggg aatctggcgg tcatttcgct cacgcgatgc | 900 |
| gtagctgcag cagaagctga ggagatgggg ctgcaggagt tggtaaatct acttggagat | 960 |
| gcgattggaa aagcgatagc tgatcatgct gaaatgttgt ctcctaatca agaagggtgt | 1020 |
| gacatcatta ttaatgattt caagaacttt ttaacattat tcgggactcc taacacaaat | 1080 |
| attattactc ttactgattg gagtaagttt ggattctacg aagctgattt tgggtttggg | 1140 |
| aagcctgttt ggaccagcag cggacagcaa tccctgagcg ttaccaccat tgtgctcatg | 1200 |
| aacaacaaag agggcgatgg aatcgaggca tggctgcatc tcaacaaaaa tgacatgctt | 1260 |
| ttcttcgagc aagatgaaga aatcaaatta tttactacct aa | 1302 |

SEQ ID NO: 16

| | |
|---|---|
| MKMLMIKSQF RVHSIVSAWA NNSNKRQSLG HQIRRKQRSQ VTECRVASLD ALNGIQKVGP | 60 |
| ATIGTPEEEN KKIEDSIEYV KELLKTMGDG RISVSPYDTA IVALIKDLEG GDGPEFPSCL | 120 |
| EWIAQN0LAD GSWGDHFFCI YDRVVNTAAC VVALKSWNVH ADKIEKGAVY LKENVHKLKD | 180 |
| GKIEHMPAGF EFVVPATLER AKALGIKGLP YDDPFIREIY SAKQTRLTKI PKGMIYESPT | 240 |
| SLLYSLDGLE GLEWDKILKL QSADGSFITS VSSTAFVFMH TNDLKCHAFI KNALTNCNGG | 300 |
| VPHTYPVDIF ARLWAVDRLQ RLGISRFFEP EIKYLMDHIN NVWREKGVFS SRHSQFADID | 360 |
| DTSMGIRLLK MHGYNVNPNA LEHFKQKDGK FTCYADQHIE SPSPMYNLYR AAQLRFPGEE | 420 |
| ILQQALQFAY NFLHENLASN HFQEKWVISD HLIDEVRIGL KMPHYATLPR VEASYYLQHY | 480 |
| GGSSDVWIGK TLYRMPEISN DTYKILAQLD FNKCQAQHQL EWNSMKEWYQ SNNVKEFGIS | 540 |
| KKELLLAYFL AAATMFEPER TQERIMWAKT QVVSRMITSF LNKENTMSFD LKIALLTOPQ | 600 |
| HQINGSEMKN GLAQTLPAAF RQLLKEFDKY TRHQLRNTWN KWLMKLKQGD DNGGADAELL | 660 |
| ANTLNICAGH NEDILSHYEY TALSSLTNKI CORLSQIQDK KMLEIEEGSI KDKEMELEIQ | 720 |
| TLVKLVLQET SGGIDRNIKQ TFLSVFKTFY YRAYHDAKTI DAHIFQVLFE PVV | 773 |

SEQ ID NO: 17

| | |
|---|---|
| MSSLAGNLRV IPFSGNRVQT RTGILPVHQT PMITSKSSAA VKCSLTTPTD LMGKIKEVFN | 60 |
| REVDTSPAAM TTHSTDIPSN LCIIDTLQRL GIDQYFQSEI DAVLHDTYRL WQLKKKDIFS | 120 |
| DITTHAMAFR LLRVKGYEVA SDELAPYADQ ERINLQTIDV PTVVELYRAA QERLTEEDST | 180 |
| LEKLYVWTSA FLKQQLLTDA IPDKKLHKQV EYYLKNYHGI LDRMGVRRNL DLYDISHYKS | 240 |
| LKAAHRFYNL SNEDILAFAR QDFNISQAQH QKELQQLQRW YADCRLDTLK FGRDVVRIGN | 300 |
| FLTSAMIGDP ELSDLRLAFA KHIVLVTRID DFFDHGGPKE ESYEILELVK EWKEKPAGEY | 360 |
| VSEEVEILFT AVYNTVNELA EMAHIEQGRS VKDLLVKLWV EILSVFRIEL DTWTNDTALT | 420 |
| LEEYLSQSWV SIGCRICILI SMQFQGVKLS DEMLQSEECT DLCRYVSMVD RLLNDVQTFE | 480 |
| KERKENTGNS VSLLQAAHKD ERVINEEEAC IKVKELAEYN RRKLMQIVYK TGTIFPRKCK | 540 |
| DLFLKACRIG CYLYSSGDEF TSPQQMMEDM KSLVYEPLPI SPPEANNASG EKMSCVSN | 598 |

SEQ ID NO: 18

| | |
|---|---|
| MSITINLRVI AFPGHGVQSR QGIFAVMEFP RNKNTFKSSF AVKCSLSTPT DLMGKIKEKL | 60 |
| SEKVDNSVAA MATDSADMPT NLCIVDSLQR LGVEKYFQSE IDTVLDDAYR LWQLKQKDIF | 120 |
| SDITTHAMAF RLLRVKGYDV SSEELAPYAD QEGMNLQTID LAAVIELYRA AQERVAEEDS | 180 |
| TLEKLYVWTS TFLKQQLLAG AIPDQKLHKQ VEYYLKNYHG ILDRMGVRKG LDLYDAGYYK | 240 |
| ALKAADRLVD LCNEDLLAFA RQDFNINQAQ HRKELEQLQR WYADCRLDKL EFGRDVVRVS | 300 |
| NFLTSAILGD PELSEVRLVF AKHIVLVTRI DDFFDHGGPR EESHKILELI KEWKEKPAGE | 360 |
| YVSKEVEILY TAVYNTVNEL AERANVEQGR NVEPFLRTLW VQILSIFKIE LDTWSDDTAL | 420 |
| TLDDYLNNSW VSIGCRICIL MSMQFIGMKL PEEMLLSEEC VDLCRHVSMV DRLLNDVQTF | 480 |
| EKERKENTGN AVSLLLAAHK GERAFSEEEA IAKAKYLADC NRRSLMQIVY KTGTIFPRKC | 540 |
| KDMFLKVCRI GCYLYASGDE FTSPQQMMED MKSLVYEPLQ IHPPPAN | 587 |

SEQ ID NO: 19

| | |
|---|---|
| MELVEVIVVV VGAAALGVVL WSHLKPEGRK LPPGPSPLPI FGNIFQLTGP NTCESFANLS | 60 |
| KKYGPVMSLR LGSLFTVVIS SPEMAKEVLT NTDFLERPLM QAVHAHDHAQ FSIAFLPVTT | 120 |
| PKWKQLRRIC QEQMFASRIL EKSQPLRHQK LQELIDHVQK CCDAGRAVTI RDAAFATTLN | 180 |
| LMSVTMFSAD ATELDSSVTA ELRELMAGVV TVLGTPNFAD FFPILKYLDP QGVRRKAHFH | 240 |
| YGKMFDHIKS RMAERVELKK ANPNHLKHDD FLEKILDISL RRDYELTIQD ITHLLVDLYV | 300 |
| AGSESTVMSI EWIMSELMLH PQSLAKLKAE LRSVMGERKM IQESEDISRL PFLNAVIKET | 360 |
| LRLHPPGPLL FPRQNTNDVE LNGYFIPKGT QILVNEWAIG RDPSVWPNPE SFVPERFLDK | 420 |
| NIDYKGQDPQ LVPFGSGRRI CLGIPIAHRM VHSTVAALIH NFEWKFAPDG SEYNRELFSG | 480 |
| PALRREVPLN LIPLNPSF | 498 |

SEQ ID NO: 20

| | |
|---|---|
| METITLLLAL FFIALTYFIS SRRRRNLPPG PFPLPIIGNM LQLGSKPHQS FAQLSKKYGP | 60 |
| LMSIHLGSLY TVIVSSPEMA KEILQKHGQV FSGRTIAQAV HACDHDKISM GFLPVANTWR | 120 |
| DMRKICKEQM FSHHSLEASE ELRHQKLQQL LDYAQKCCEA GRAVDIREAS FITTLNLMSA | 180 |
| TMFSTQATEF DSEATKEFKE IIEGVATIVG VANFADYFPI LKPFDLQGIK RRADGYFGRL | 240 |
| LKLIEGYLNE RLESRRLNPD APRKKDFLET LVDIIEANEY KLTTEHLTHL MLDLFVGGSE | 300 |
| TNTTSLEWIM SELVINPDKM AKVKEELKSV VGDEKLVNES DMPRLPYLQA VIKEVLRIHP | 360 |
| PGPLLLPRKA ESDQVVNGYL IPKGTOILFN AWAMGRDPTI WKDPESFEPE RFLNQSIDFK | 420 |
| GQDFELIPFG SGRRICPGMP LANRILHMTT ATLVHNFDWK LEEGTADADH KGELFGLAVR | 480 |
| RATPLRIIPL KP | 492 |

SEQ ID NO: 21

| | |
|---|---|
| MELVQVIAVV AVVVVLWSQL KRKGRKLPPG PSPLPIVGNI FQLSGKNINE SFAKLSKIYG | 60 |
| PVMSLRLGSL LTVIISSPEM AKEVLTSKDF ANRPLTEAAH AHGHSKFSVG FVPVSDPKWK | 120 |
| QMRRVCQEEM FASRILENSQ QRRHQKLQEL IDHVQESRDA GRAVTIRDPV FATTLNIMSL | 180 |
| TLFSADATEF SSSATAELRD IMAGVVSVLG AANLADFFPI LKYFDPQGMR RKADLHYGRL | 240 |
| IDHIKSRMDK RSELKKANPN HPKHDDFLEK IIDITIQRNY DLTINEITHL LVDLYLAGSE | 300 |
| STVMTIEWTM AELMLRPESL AKLKAELRSV MGERKMIQES DDISRLPYLN GAIKEALRLH | 360 |
| PPGPLLFARK SEIDVELSGY FIPKGTQILV NEWGMGRDPS VWPNPECFQP ERFLDKNIDY | 420 |
| KGQDPQLIPF GAGRRICPGI PIAHRVVHSV VAALVHNFDW EFAPGGSQCN NEFFTGAALV | 480 |
| REVPLKLIPL NPPSI | 495 |

SEQ ID NO: 22

| | |
|---|---|
| METMTLLLPL FFIALTYFLS WRRRRNLPPG PFPLPIIGNL LQIGSKPHQS FAQLSKKYGP | 60 |
| LMSVQLGSVY TVIASSPEMA KEILOKHGQV FSGRTIAQAA QACGHDQISI GFLPVATTWR | 120 |
| DMRKICKEQM FSHHSLESSK ELRHEKLQKL LDYAQKCCEA GRAVDIREAA FITTLNLMSA | 180 |
| TLFSTQATEF DSEATKEFKE VIEGVAVIVG EPNFADYFPI LKPFDLQGIK RRANSYFGRL | 240 |
| LKLMERYLNE RLESRRLNPD APKKNDFLET LVDIIQADEY KLTTDHVTHL MLDLFVGGSE | 300 |
| TSATSLEWIM SELVSNPSKL AKVKAELKSV VGEKKVVSES EMARLPYLQA VIKEVLRLHP | 360 |
| PGPLLLPRKA GSDQVVNGYL IPKGTQLLFN VWAMGRDPSI WKNPESFEPE RFLNQNIDYK | 420 |
| GQDFELIPFG SGRRICPGMP LADRIMHMTT ATLVHNFDWK LEDGAGDADH KGDDPFGLAI | 480 |
| RRATPLRIIP LKP | 493 |

SEQ ID NO: 23

| | |
|---|---|
| MESMNALVVG LLLIALTILF SLRRRRNLAP GPYPFPIIGN MLQLGTKPHQ SFAQLSKKYG | 60 |
| PLMSIHLGSL YTVIVSSPEM AKEILQKHGQ VFSGRTIAQA VHACDHDKIS MGFLPVSNTW | 120 |
| RDMRKICKEQ MFSHHSLEGS QGLRQQKLLQ LLDYAQKCCE TGRAVDIREA SFITTLNLMS | 180 |
| ATMFSTQATE FESKSTQEFK EIIEGVATIV GVANFGDYFP ILKPFDLQGI KRKADGYFGR | 240 |
| LLKLIEGYLN ERLESRKSNP NAPRKNDFLE TVVDILEANE YKLSVDHLTH LMLDLFVGGS | 300 |
| ETNTTSLEWT MSELVNNPDK MAKLKQELKS VVGERKLVDE SEMPRLPYLQ AVIKESLRIH | 360 |
| PPGPLLLPRK AETDQEVNGY LIPKGTQILF NVWAMGRDPS IWKDPESFEP ERFLNQNIDF | 420 |
| KGQDFELIPF GSGRRICPGM PLANRILHMA TATMVHNFDW KLEQGTDEAD AKGELFGLAV | 480 |
| RRAVPLRIIP LQP | 493 |

SEQ ID NO: 24

| | |
|---|---|
| MKVERFSRKF IKPHTPTPEN LKKYKLSLLD KCLGHDNFAI VLFYESKPRN KSELEESLEK | 60 |
| VLVDFYPLAG RHTMNDHIVD CSDVGAVFVE AEALDVELTM DELVKNMEAQ TIHHLLPNQY | 120 |
| FSADAPNPLL SIQVTHFPSG GLAIGIAVSH AVFDGFSLGV FVAAWSKATM NPDRKIKITP | 180 |
| SFDLPSLLPY KDDNFGLTAA EIVSQSEDIV VKRFIFGKEA ITRLRSKLSP NRNGKKISRV | 240 |
| RVVCAVIVKA LMGLERAKTR DFMICQGINM RERTKAPLQK HACGNLAVSS YTRRVAAAEA | 300 |
| EELQSLVNLI GDSIEKSIAD YADILSSDQD GRHIISTMMK SFMQFAAPDI KAISFTDWSK | 360 |
| FGFYQVDFGF GKPVWTGVRP ERPIFSAAIL MSNREGDGIE AWLHLDKNDM LIFEQDEEIK | 420 |
| LFTT | 424 |

SEQ ID NO: 25

| | |
|---|---|
| atgaaggtgg aaagatttag caggaaattc ataaaaccac acaccccgac ccccgaaaat | 60 |
| ctgaagaagt ataagctttc tcttttagac aaatgcttgg ggcacgataa ttttgctatt | 120 |
| gttctgtttt acgaatcgaa accaagaaac aagagtgagt tggaagaatc actggaaaaa | 180 |
| gtcctggtgg atttctaccc tcttgctgga agacacacca tgaatgatca catagttgat | 240 |
| tgcagtgatg tgggcgccgt gtttgtcgaa gcagaagctc tagacgtcga gctgacgatg | 300 |
| gatgagctcg ttaagaacat ggaggcccag actatccatc atctccttcc caatcaatat | 360 |
| ttctcagctg atgctccaaa tccactcttg tcaatccagg tgacacattt cccatctggt | 420 |
| ggcctagcaa ttggcatcgc cgtttctcac gctgtcttcg atggtttttc gctgggggtg | 480 |
| ttcgtcgccg cctggtcgaa ggccaccatg aacccagatc ggaagatcaa aatcacaccg | 540 |
| tctttcgatc ttccctcgtt gcttccttac aaggatgaca attttggatt aactgccgcc | 600 |
| gaaattgtca gccagagtga agatattgtt gttaagaggt ttattttcgg gaaggaggca | 660 |
| ataacgaggt tgagatcaaa gctaagccca aatcgcaatg ggaaaaagat ctctcgtgtt | 720 |
| cgagtggtgt gtgccgttat agtgaaagcc ttgatgggcc tggaacgcgc caaaacaaga | 780 |
| gatttcatga tctgtcaagg gatcaacatg cgtgagagaa caaaggcacc tctacagaag | 840 |
| catgcttgtg ggaatctggc ggtctcgtct tacacgcgac gtgtagccgc agcagaagcc | 900 |
| gaggagctgc agagtttggt aaatctaata ggtgattcta ttgagaagag catagctgat | 960 |
| tatgctgata ttctttcttc tgatcaagat gggcgtcata tcatcagcac gatgatgaaa | 1020 |
| tctttcatgc aattcgcggc tcctgacata aaagctatat ccttcactga ttggagtaag | 1080 |
| tttggattct accaagttga ttttgggttc gggaagcctg tttggacggg tgttcggccc | 1140 |
| gaacgcccaa tctttagcgc cgctattctc atgagcaaca gagagggtga tggaattgag | 1200 |
| gcatggcttc atcttgacaa aaatgacatg cttatcttcg agcaagatga agaaatcaaa | 1260 |
| ttatttacta cctaa | 1275 |

SEQ ID NO: 26

| | |
|---|---|
| MKVERISRKF IKPYTPTPQN LKKYKLSLLD KCMGHMDFAV VLFYESKPRN KNELEESLEK | 60 |
| VLVDFYPLAG RYTMNDHIVD CSDEGAVFVE AEAPNVELTV DQLVKNMEAQ TIHDFLPDQY | 120 |
| FPADAPNPLL SIQvTHFPCG GLAIGIVVSH AVFDGFSLGV FLAAWSKATM NPERKIEITP | 180 |
| SFDLPSLLPY KDESFGLNFS EIVKAENIVV KRLNFGKEAI TRLRSKLSPN QNGKTISRVR | 240 |
| VVCAVIVKAL MGLERAKTRD FMICQGINMR ERTKAPLQKH ACGNLAVSSY TRRVAAAEAE | 300 |
| ELQSLVNLIG DSIEKSIADY ADILSSDQDG RHIISTMMKS FMQFAAPDIK AISFTDWSKF | 360 |
| GFYQVDFGFG KPVWTGVRPE RPIFSAAILM SNREGDGIEA WLHLDKNDML IFEQDEEIKL | 420 |
| LITT | 424 |

SEQ ID NO: 27

| | |
|---|---|
| atgaaggttg aaagaataag caggaaattc ataaaaccat acaccccaac cccccaaaac | 60 |
| ctcaagaaat ataagctttc tcttttagac aaatgcatgg ggcacatgga ttttgctgtt | 120 |
| gttctgtttt atgaatcgaa accaagaaac aagaatgaat tggaagaatc actagaaaaa | 180 |
| gtcctggtcg atttctaccc tcttgctgga agatacacca tgaatgatca catagttgat | 240 |
| tgcagtgatg agggcgccgt gtttgtcgaa gccgaagctc caaacgtcga gctgacggtg | 300 |
| gatcaactcg tcaagaacat ggaggcccag actatccatg atttccttcc cgatcaatat | 360 |
| ttcccagctg atgctccaaa tccactcctc tcgatccagg tcacgcattt cccatgtggt | 420 |
| ggcttggcga ttggcatcgt tgtttctcac gctgtcttcg atggtttttc gctgggggtg | 480 |
| ttccttgccg cctggtcgaa ggccaccatg aacccagaga ggaagatcga aatcaccccg | 540 |
| tctttcgatc ttccttcgtt gcttccatac aaggatgaaa gtttcggatt aaatttcagc | 600 |
| gaaattgtca aggctgaaaa tattgttgtg aagaggctta atttcgggaa ggaggctata | 660 |
| acgaggttga gatcaaaact aagcccaaat caaaatggga aaacgatctc tcgcgttcga | 720 |
| gttgtttgcg ccgttatagt gaaagccttg atgggcctgg aacgcgccaa aacaagagat | 780 |
| ttcatgatct gtcaagggat caacatgcgt gagagaacaa aggcacctct acagaagcat | 840 |
| gcttgtggga atctggcggt ctcgtcttac acgcgacgtg tagccgcagc agaagccgag | 900 |
| gagctgcaga gtttggtaaa tctaataggt gattctattg agaagagcat agctgattat | 960 |
| gctgatattc tttcttctga tcaagatggg cgtcatatca tcagcacgat gatgaaatct | 1020 |
| ttcatgcaat tcgcggctcc tgacataaaa gctatatcct tcactgattg gagtaagttt | 1080 |
| ggattctacc aagttgattt tgggttcggg aagcctgttt ggacgggtgt tcggcccgaa | 1140 |
| cgcccaatct ttagcgccgc tattctcatg agcaacagag agggtgatgg aattgaggca | 1200 |
| tggcttcatc ttgacaaaaa tgacatgctt atcttcgagc aagatgaaga aatcaaatta | 1260 |
| ttaattacta cctaa | 1275 |

SEQ ID NO: 28

| | |
|---|---|
| atgaaggtcg aaagaatctc cagaaagttc attaagccat acactccaac tccacaaaac | 60 |
| ttgaagaagt acaagttgtc cttgttggat aagtgcatgg gtcatatgga tttcgctgtt | 120 |
| gttttgttct acgaatccaa gccaagaaac aagaacgaat tggaagaatc cttggaaaag | 180 |
| gttttggttg acttttatcc attggctggt agatacacca tgaacgatca tatagttgat | 240 |
| tgctctgatg aaggtgccgt ttttgttgaa gctgaagctc caaatgttga attgaccgtt | 300 |
| gatcaattgg tcaagaacat ggaagctcaa accatccatg atttcttgcc agatcaatac | 360 |
| tttccagctg atgctcctaa tcctttgttg tctattcaag ttacccattt cccatgtggt | 420 |
| ggtttggcta ttggtatagt tgtttctcat gctgttttcg acggtttctc tttgggtgtt | 480 |
| tttttggctg cttggtctaa ggctactatg aatccagaaa gaaagatcga aatcacccca | 540 |
| tcttttgact tgccatcttt gttgccttac aaggatgaat ctttcggttt gaacttctcc | 600 |
| gaaatcgtta aggctgaaaa catcgttgtc aagagattga acttcggtaa agaagccatt | 660 |
| accagattga gatctaagtt gtccccaaat caaaacggta agaccatctc tagagttaga | 720 |
| gttgtttgtg ccgttattgt caaggctttg atgggtttgg aaagagctaa gactagagat | 780 |
| ttcatgatct gccaaggtat caacatgaga gaaagaacaa aagccccatt gcaaaaacat | 840 |
| gcttgtggta atttggccgt ttcttcatac actagaagag ttgctgctgc tgaagcagaa | 900 |
| gaattgcaat ctttggttaa cttgatcggt gactccatcg aaaagtctat tgctgattac | 960 |
| gccgatatct tgtcctctga tcaagatggt agacatatca tctccaccat gatgaagtct | 1020 |
| ttcatgcaat ttgctgcccc agatattaag gctatttctt tcactgactg gtccaagttt | 1080 |
| ggtttctacc aagttgattt tggtttcggt aaaccagttt ggactggtgt tagaccagaa | 1140 |
| agaccaattt tttccgctgc cattttgatg tctaacagag aaggtgatgg tattgaagct | 1200 |
| tggttgcatt tggataagaa cgacatgttg atcttcgaac aagacgaaga aatcaagttg | 1260 |
| ttgatcacca cctga | 1275 |

SEQ ID NO: 29

| | |
|---|---|
| atggcgtctt gtggagctat cgggagtagt ttcttgccac tgctccattc cgacgagtca | 60 |
| agcttgttat ctcggcccac tgctgctctt cacatcaaga agcagaagtt ttctgtggga | 120 |
| gctgctctgt accaggataa cacgaacgat gtcgttccga gtggagaggg tctgacgagg | 180 |
| cagaaaccaa gaactctgag tttcacggga gagaagcctt caactccaat tttggatacc | 240 |
| atcaactatc caatccacat gaagaatctg tccgtggagg aactggagat attggccgat | 300 |
| gaactgaggg aggagatagt ttacacggtg tcgaaaacgg gagggcattt gagctcaagc | 360 |
| ttgggtgtat cagagctcac cgttgcactg catcatgtat tcaacacacc cgatgacaaa | 420 |
| atcatctggg atgttggaca tcaggcgtat ccacacaaaa tcttgacagg gaggaggtcc | 480 |
| agaatgcaca ccatccgaca gactttcggg cttgcagggt tccccaagag ggatgagagc | 540 |
| ccgcacgacg cgttcggagc tggtcacagc tccactagta tttcagctgg tctagggatg | 600 |
| gcggtgggga gggacttgct acagaagaac aaccacgtga tctcggtgat cggagacgga | 660 |
| gccatgacag cggggcaggc atacgaggcc atgaacaatg caggatttct tgattccaat | 720 |
| ctgatcatcg tgttgaacga caacaaacaa gtgtccctgc ctacagccac cgtcgacggc | 780 |
| cctgctcctc ccgtcggagc cttgagcaaa gccctcacca agctgcaagc aagcaggaag | 840 |
| ttccggcagc tacgagaagc agcaaaaggc atgactaagc agatgggaaa ccaagcacac | 900 |
| gaaattgcat ccaaggtaga cacttacgtt aaaggaatga tggggaaacc aggcgcctcc | 960 |
| ctcttcgagg agctcgggat ttattacatc ggccctgtag atggacataa catcgaagat | 1020 |
| cttgtctata ttttcaagaa agttaaggag atgcctgcgc ccggccctgt tcttattcac | 1080 |
| atcatcaccg agaagggcaa aggctaccct ccagctgaag ttgctgctga caaaatgcat | 1140 |
| ggtgtggtga agtttgatcc aacaacgggg aaacagatga aggtgaaaac gaagactcaa | 1200 |
| tcatacaccc aatacttcgc ggagtctctg gttgcagaag cagagcagga cgagaaagtg | 1260 |
| gtggcgatcc acgcggcgat gggaggcgga acggggctga acatcttcca gaaacggttt | 1320 |
| cccgaccgat gtttcgatgt cgggatagcc gagcagcatg cagtcacctt cgccgcgggt | 1380 |
| cttgcaacgg aaggcctcaa gcccttctgc acaatctact cttccttcct gcagcgaggt | 1440 |
| tatgatcagg tggtgcacga tgtggatctt cagaaactcc cggtgagatt catgatggac | 1500 |
| agagctggac ttgtgggagc tgacggccca acccattgcg gcgccttcga caccacctac | 1560 |
| atggcctgcc tgcccaacat ggtcgtcatg gctccctccg atgaggctga gctcatgcac | 1620 |
| atggtcgcca ctgccgctgt cattgatgat cgccctagct gcgttaggta ccctagagga | 1680 |
| aacggtatag gggtgcccct ccctccaaac aataaaggaa ttccattaga ggttgggaag | 1740 |
| ggaaggattt tgaaagaggg taaccgagtt gccattctag gcttcggaac tatcgtgcaa | 1800 |
| aactgtctag cagcagccca acttcttcaa gaacacggca tatccgtgag cgtagccgat | 1860 |
| gcgagattct gcaagcctct ggatggagat ctgatcaaga atcttgtgaa ggagcacgaa | 1920 |
| gttctcatca ctgtggaaga gggatccatt ggaggattca gtgcacatgt ctctcatttc | 1980 |
| ttgtccctca atggactcct cgacggcaat cttaagtgga ggcctatggt gctcccagat | 2040 |
| aggtacattg atcatggagc ataccctgat cagattgagg aagcagggct gagctcaaag | 2100 |
| catattgcag gaactgtttt gtcacttatt ggtggaggga aagacagtct tcatttgatc | 2160 |
| aacatgtaa | 2169 |

SEQ ID NO: 30

| | |
|---|---|
| MASCGAIGSS FLPLLHSDES SLLSRPTAAL HIKKQKFSVG AALYQDNTND VVPSGEGLTR | 60 |
| QKPRTLSFTG EKPSTPILDT INYPIHMKNL SVEELEILAD ELREEIVYTV SKTGGHLSSS | 120 |
| LGVSELTVAL HHVFNTPDDK IIWDVGHQAY PHKILIGRRS RMHTIRQTFG LAGFPKRDES | 180 |
| PHDAFGAGHS STSISAGLGM AVGRDLLQKN NHVISVIGDG AMTAGOAYEA MNNAGFLDSN | 240 |
| LIIVLNDNKQ VSLPTATVDG PAPPVGALSK ALTKLQASRK FRQLREAAKG MTKQMGNQAH | 300 |
| EIASKVDTYV KGMMGKPGAS LFEELGIYYI GPVDGHNIED LVYIFKKVEE MPAPGPVLIH | 360 |
| IITEKGKGYP PAEVAADKMH GVVKFDPTTG KQMKVKTKTQ SYTQYFAESL VAEAEQDEKV | 420 |
| VAIHAAMGGG TGLNIFQKRF PDRCFDVGIA EQHAVTFAAG LATEGLKPFC TIYSSFLQRG | 480 |
| YDQVVHDVDL QKLPVRFMMD RAGLVGADGP THCGAFDTTY MACLPNMVVM APSDEAELMH | 540 |
| MVATAAVIDD RPSCVRYPRG NGIGVPLPPN NKGIPLEVGK GRILKEGNRV AILGFGTIVQ | 600 |
| NCLAAAQLLO EHGISVSVAD ARFCKPLDGD LIKNLVREHE VLITVEEGSI GGFSAHVSHF | 660 |
| LSLNGLLDGN LKWRPMVLPD RYIDHGAYPD QIEEAGLSSK HIAGTVLSLI GGGKDSLHLI | 720 |
| NM | 722 |

SEQ ID NO: 31

| | |
|---|---|
| atgaggtcta tgaatctggt cgatgcttgg gttcaaaacc tccccatttt caagcaacca | 60 |
| cacccctcca aattcatcca ccatcccaga ttcgagcccg ctttcctcaa atcgcggagg | 120 |
| cccatttcct ccttcgccgt ctccgccgtc ctcaccggcg aggaagcaag aatcttcacc | 180 |
| cgaggagatg aagcgccctt caatttcaac gcctacgtcg tcgagaaagc cacccacgtg | 240 |
| aacaaggctc tcgacgacgc ggtggcggtg aagaaccctc cgatgatcca cgaggccatg | 300 |
| aggtactcct tgctcgccgg cggaaagagg gtccgcccca tgctctgcat cgccgcctgc | 360 |
| gaggtggtgg gcggccccca agcggcggcg atccccgccg cctgcgcggt ggagatgatc | 420 |
| cacaccatgt ctctcatcca cgatgatctt ccctgtatgg acaatgatga cctccgccgc | 480 |
| ggcaagccca ccaatcacaa agtcttcggc gagaacgtcg ccgtgctcgc cggtgatgct | 540 |
| ttattggcct tcgcgtttga attcatcgcc actgccacca cgggggtggc ccctgagagg | 600 |
| attcttgcgg cggtggcgga gttggcgaag gcgatcggga cggaggggct ggtggcgggg | 660 |
| caggtggtgg atttgcattg caccggcaat cccaatgtag gactggacac attggaattc | 720 |
| atacacatac acaaaactgc agcattgctt gaggcctctg tagttttggg ggccattttg | 780 |
| ggaggaggaa gcagtgatca agttgagaaa ctgagaactt ttgctagaaa aattgggctt | 840 |
| ctcttccaag tggtggatga cattttagat gtcacaaaat cctcggagga gttggggaag | 900 |
| acggccggca aagacttggc cgtcgacaag accacctacc caaagcttct gggattggag | 960 |
| aaagctatgg agtttgctga gaggctgaat gaggaggcca agcagcagct gctggatttt | 1020 |
| gacccccgga aggcggcgcc gctggtggcg ctggccgatt acattgctca caggcagaac | 1080 |
| tag | 1083 |

SEQ ID NO: 32

| | |
|---|---|
| MRSMNLVDAW VQNLPIFKQP HPSKFIHHPR FEPAFLKSRR PISSFAVSAV LTGEEARIFT | 60 |
| RGDEAPFNFN AYVVEKATHV NKALDDAVAV KNPPMIHEAM RYSLLAGGKR VRPMLCIAAC | 120 |
| EVVGGPQAAA IPAACAVEMI HTMSLIHDDL PCMDNDDLRR GKPTNHKVFG ENVAVLAGDA | 180 |
| LLAFAFEFIA TATTGVAPER ILAAVAELAK AIGTEGLVAG QVVDLHCTGN PNVGLDTLEF | 240 |
| IHIHKTAALL EASVVLGAIL GGGSSDQVEK LRTFARKIGL LFQVVDDILD VTKSSEELGK | 300 |
| TAGKDLAVDK TTYPKLLGLE KAMEFAERLN EEAKQQLLDF DPRKAAPLVA LADYIAHRQN | 360 |

SEQ ID NO: 33

| | |
|---|---|
| atggaatcga ctattgagaa gctttcgccc ttcgatttga tgactgcgat tctcaaagga | 60 |
| gtcaaacttg ataattcgaa cgggtctgct ggggtggagc atccggctgt gatcgcgatg | 120 |
| ctgatggaga acaaggatct cgtgatgatg ctcaccacct ccgtcgcggt gcttctagga | 180 |
| cttgctgtgt atctcgtgtg gcggcgcgga gccggatcgg cgaagagggt ggtggagccg | 240 |
| ccgaagctgg tgattcccaa gggcccggtg gatgcggagg aagaggatga tgggaagaag | 300 |
| aaggttacca tcttttttgg gacgcagact ggaactgctg aaggctttgc taaggcactt | 360 |
| gccgaagaag ctaaagcaag atatccgctg accaacttta aagtagttga cttggatgat | 420 |
| tatgctgccg atgatgaaga gtatgaagag aagatgaaga aggagacctt tgcattcttc | 480 |
| ttcttggcga catatggaga tggtgagcct accgacaatg ctgcgagatt ttacaagtgg | 540 |
| ttttccgagg ggaaagagag aggtgagata ttcaagaatc tcaactatgg tgtatttggt | 600 |
| cttggaaaca ggcagtatga gcatttcaac aagattgcta tagtggtgga tgacattctt | 660 |
| cttgagcaag gtggaaatcg gcttgtccct gtgggtcttg gagatgacga tcaatgtatc | 720 |
| gaagatgatt tctcagcatg gcgtgataat gtgtggcctg agctggataa gttgctccgt | 780 |
| gatgaggatg atgcaactgt tgcaactcca tatactgcag ccgttttgga gtatcgtgtt | 840 |
| gtgttccatg accagtcaga tgaactgcac tcggaaaaca acttagccaa tggtcatgca | 900 |
| aatggaaatg cttcttatga tgctcaacac ccctgcaaag tgaatgttgc tgtaaaaagg | 960 |
| gagctacata ctcctctatc cgatcgttct tgcactcact tggaattcga catatctggc | 1020 |
| actggattag agtatgaaac aggggaccac gttggtgttt actgtgagaa cttgattgaa | 1080 |
| actgtagagg aagcagaaag gcttcttggt ctttctccac aaacattctt ttcagttcac | 1140 |
| actgataaag cggacggcac accacttggt ggaagtgcct tgcctcctcc cttcccgccg | 1200 |
| tgcactttga ggacagcgct aagtcgatat gctgatcttt tgaatgctcc caaaaagtct | 1260 |
| gctttgactg cattggctgc ttatgcctct gaccctagtg aagctgatcg gctcaagcac | 1320 |
| cttgcttccc ctgatggaaa ggaggaatat gctcaatatg tggtttctgg tcagagaagc | 1380 |
| ctacttgagg tgatggctga cttcccatct gccaagcctc ctcttggtgt tttctttgct | 1440 |
| gcaattgctc ctcgcttgca gcctcgattt tattcaatct catcctcacc aaagattgca | 1500 |
| ccttcaagaa ttcacgtcac ttgtgcgttg gtgtatgaga aaatgcccac tggacgaatc | 1560 |
| cacaagggtg tctgctcaac atggatgaag aatgctgtgc cattggagga aagccccaac | 1620 |
| tgctcttcag caccagtttt tgtacggacc tcaaacttca gactccctgc tgatcctaaa | 1680 |
| gtaccagtta taatgattgg ccctggaacc ggtttggctc cattcagggg ttttcttcag | 1740 |
| gaaagattag ccctcaagga atctggagca gaacttggtc ctgctatatt attcttcggg | 1800 |
| tgcagaaaca gtaaaatgga tttcatttac caagatgaac tggataactt tgttaaagct | 1860 |
| ggagtggttt ctgagcttgt ccttgcgttt tcacgcgagg gtcctgctaa ggaatacgtg | 1920 |
| cagcataaga tggcacagaa ggcctcggat gtgtggaata tgatatcaga agggggctac | 1980 |
| gtttatgtat gtggtgatgc taagggcatg gcacgtgacg ttcaccggac tcttcacacc | 2040 |
| attgttcaag aacagggatc tctggacagc tcgaaaaccg agagcttcgt caagaatctg | 2100 |
| cagatgaccg gccggtacct gcgtgacgtg tggtga | 2136 |

SEQ ID NO: 34

| | |
|---|---|
| MESTIEKLSP FDLMTAILKG VKLDNSNGSA GVEHPAVIAM LMENKDLVMM LTTSVAVLLG | 60 |
| LAVYLVWRRG AGSAKRVVEP PKLVIPKGPV DAEEEDDGKK KVTIFFGTQT GTAEGFAKAL | 120 |
| AEEAKARYPL TNFKVVDLDD YAADDEEYEE KMKKETFAFF FLATYGDGEP TDNAARFYKW | 180 |
| FSEGKERGEI FKNLNYGVFG LGNRQYEHFN KIAIVVDDIL LEQGGNRLVP VGLGDDDQCI | 240 |
| EDDFSAWRDN VWPELDKLLR DEDDATVATP YTAAVLEYRV VFHDQSDELH SENNLANGHA | 300 |
| NGNASYDAQH PCKVNVAVKR ELHTPLSDRS CTHLEFDISG TGLEYETGDH VGVYCENLIE | 360 |
| TVEEAERLLG LSPQTFFSVH TDKADGTPLG GSALPPPFPP CTLRTALSRY ADLLNAPKKS | 420 |
| ALTALAAYAS DPSEADRLKH LASPDGKEEY AQYVVSGQRS LLEVMADFPS AKPPLGVFFA | 480 |
| AIAPRLQPRF YSISSSPKIA PSRIHVTCAL VYEKMPTGRI HKGVCSTWMK NAVPLEESPN | 540 |
| CSSAPVFVRT SNFRLPADPK VPVIMIGPGT GLAPFRGFLQ ERLALKESGA ELGPAILFFG | 600 |
| CRNSKMDFIY QDELDNFVKA GVVSELVLAF SREGPAKEYV QHKMAQKASD VWNMISEGGY | 660 |
| VYVCGDAKGM ARDVHRTLHT IVQEQGSLDS SKTESFVKNL QMTGRYLRDV W | 711 |

SEQ ID NO: 35

| | |
|---|---|
| atgtccagag ttgcttcctt ggatgctttg aatggtattc aaaaagttgg tccagctacc | 60 |
| attggtactc cagaagaaga aaacaagaag atcgaagatt ccatcgaata cgtcaaagaa | 120 |
| ttattgaaaa ccatgggtga cggtagaatc tctgtttctc catatgatac tgctatcgtc | 180 |
| gccttgatta aggatttgga aggtggtgat ggtccagaat ttccatcttg tttggaatgg | 240 |
| attgcccaaa atcaattggc tgatggttct tggggtgatc attttttctg tatctacgat | 300 |
| agagttgtta acaccgctgc ttgtgttgtt gctttgaaat cttggaatgt tcacgccgat | 360 |
| aagattgaaa aaggtgccgt ttacttgaaa gaaaacgtcc acaaattgaa ggacggtaag | 420 |
| atagaacata tgccagctgg ttttgaattc gttgttccag caactttgga aagagctaaa | 480 |
| gctttgggta ttaagggttt gccatatgat gatccattca tcagagaaat ctactccgct | 540 |
| aagcaaacta gattgactaa gattccaaag ggtatgatct acgaatctcc aacctctttg | 600 |
| ttgtactctt tggatggttt agaaggtttg gaatgggata agatcttgaa gttgcaatca | 660 |
| gctgacggtt ctttcatcac ttctgtttct tctactgcct tcgttttcat gcataccaac | 720 |
| gatttgaagt gccatgcctt tattaagaac gctttgacta actgtaatgg tggtgttcca | 780 |
| catacttacc cagttgatat ttttgctaga ttgtgggccg ttgacagatt gcaaagattg | 840 |
| ggtatttcta gattcttcga accagaaatc aaatacttga tggaccacat caacaacgtt | 900 |
| tggagagaaa agggtgtttt ctcatccaga cattctcaat tcgccgatat tgatgatacc | 960 |
| tccatgggta tcagattatt gaagatgcat ggttacaacg ttaacccaaa cgctttggaa | 1020 |
| catttcaagc aaaaggatgg taaattcacc tgttacgccg atcaacatat tgaatctcca | 1080 |
| tctccaatgt ataacttgta cagagctgcc caattgagat ttccaggtga agaaatttta | 1140 |
| caacaagcct tgcaattcgc ctacaacttc ttgcacgaaa atttggcttc taaccacttc | 1200 |
| caagaaaagt gggttatctc cgatcatttg atcgatgaag ttagaatcgg tttgaaaatg | 1260 |
| ccatggtatg ctactttgcc aagagttgaa gcttcttact acttgcaaca ttacggtggt | 1320 |
| tcttccgatg tttggattgg taaaaccttg tatagaatgc cagaaatctc taacgacacc | 1380 |
| tacaagattt tggctcaatt ggatttcaac aagtgccaag ctcaacatca attagaatgg | 1440 |
| atgtctatga aggaatggta tcaatccaac aacgtaaaag aattcggtat ctccaagaaa | 1500 |
| gaattgttgt tggcttactt tttggctgct gctactatgt ttgaacctga aagaactcaa | 1560 |
| gaaagaatca tgtgggctaa gacccaagtt gtttctagaa tgattacctc attcttgaac | 1620 |
| aaagaaaaca ctatgtcctt cgacttgaag attgctttgt tgactcaacc acaacaccaa | 1680 |
| atcaatggtt ccgaaatgaa gaatggtttg gcacaaactt taccagctgc cttcagacaa | 1740 |
| ttattgaaag aattcgacaa gtacaccaga caccaattga gaaatacttg gaacaagtgg | 1800 |
| ttgatgaagt tgaagcaagg tgatgataac ggtggtgctg atgctgaatt attggctaac | 1860 |
| actttgaaca tttgcgccgg tcataacgaa gatattttgt cccattacga atacaccgcc | 1920 |
| ttgtcatctt tgaccaacaa gatttgtcaa agattgtccc aaatccaaga taagaagatg | 1980 |
| ttggaaatcg aagaaggttc catcaaggac aaagaaatgg aattggaaat tcaaaccttg | 2040 |
| gtcaagttgg tattgcaaga aacttctggt ggtatcgaca gaaacatcaa gcaaactttc | 2100 |
| ttgtccgttt tcaagacctt ctactacaga gcttaccatg atgctaagac cattgatgcc | 2160 |
| catatcttcc aagttttgtt cgaacctgtt gtttaa | 2196 |

SEQ ID NO: 36

| | |
|---|---|
| atgatcacct ccaaatcttc cgctgctgtt aagtgttctt tgactactcc aactgatttg | 60 |
| atgggtaaga tcaaagaagt tttcaacaga gaagttgata cctctccagc tgctatgact | 120 |
| actcattcta ctgatattcc atccaacttg tgcatcatcg ataccttgca aagattgggt | 180 |
| atcgaccaat acttccaatc cgaaattgat gctgtcttgc atgatactta cagattgtgg | 240 |
| caattgaaga agaaggacat cttctctgat attaccactc atgctatggc cttcagatta | 300 |
| ttgagagtta agggttacga agttgcctct gatgaattgg ctccatatgc tgatcaagaa | 360 |
| agaatcaact tgcaaaccat tgatgttcca accgtcgtcg aattatacag agctgcacaa | 420 |
| gaaagattga ccgaagaaga ttctaccttg gaaaagttgt acgtttggac ttctgctttc | 480 |
| ttgaagcaac aattattgac cgatgccatc ccagataaga agttgcataa gcaagtcgaa | 540 |
| tattacttga agaactacca cggtatcttg gatagaatgg gtgttagaag aaacttggac | 600 |
| ttgtacgata tctcccacta caaatctttg aaggctgctc atagattcta caacttgtct | 660 |
| aacgaagata ttttggcctt cgccagacaa gatttcaaca tttctcaagc ccaacaccaa | 720 |
| aaagaattgc aacaattgca aagatggtac gccgattgca gattggatac tttgaaattc | 780 |
| ggtagagatg tcgtcagaat cggtaacttt ttaacctctg ctatgatcgg tgatccagaa | 840 |
| ttgtctgatt tgagattggc ttttgctaag cacatcgttt tggttaccag aatcgatgat | 900 |
| ttcttcgatc atggtggtcc aaaagaagaa tcctacgaaa ttttggaatt ggtcaaagaa | 960 |
| tggaaagaaa agccagctgg tgaatacgtt tctgaagaag tcgaaatctt attcaccgct | 1020 |
| gtttacaaca ccgttaacga attggctgaa atggcccata ttgaacaagg tagatctgtt | 1080 |
| aaggatttgt tggttaagtt gtgggtcgaa atattgtccg ttttcagaat cgaattggat | 1140 |
| acctggacta acgatactgc tttgactttg gaagaatact tgtcccaatc ctgggtttct | 1200 |
| attggttgca gaatctgcat tttgatctcc atgcaattcc aaggtgttaa gttgagtgac | 1260 |
| gaaatgttgc aaagtgaaga atgtaccgat ttgtgcagat acgtttccat ggtcgataga | 1320 |
| ttattgaacg atgtccaaac cttcgaaaaa gaaagaaaag aaaacaccgg taactccgtt | 1380 |
| tctttgttgc aagctgctca caaagacgaa agagttatca acgaagaaga agcctgcatc | 1440 |
| aaggtaaaag aattagccga atacaataga agaaagttga tgcaaatcgt ctacaagacc | 1500 |
| ggtactattt tcccaagaaa atgcaaggac ttgttcttga aggcttgtag aattggttgc | 1560 |
| tacttgtact cttctggtga tgaattcact tccccacaac aaatgatgga agatatgaag | 1620 |
| tccttggtct atgaaccatt gccaatttct ccacctgaag ctaacaatgc atctggtgaa | 1680 |
| aaaatgtcct gcgtcagtaa ctga | 1704 |

SEQ ID NO: 37

| | |
|---|---|
| MVAQTFNLDT YLSQRQQQVE EALSAALVPA YPERIYEAMR YSLLAGGKRL RPILCLAACE | 60 |
| LAGGSVEQAM PTACALEMIH TMSLIHDDLP AMDNDDFRRG KPTNHKVFGE DIAILAGDAL | 120 |
| LAYAFEHIAS QTRGVPPQLV LQVIARIGHA VAATGLVGGQ VVDLESEGKA ISLETLEYIH | 180 |
| SHKTGALLEA SVVSGGILAG ADEELLARLS HYARDIGLAF QIVDDILDVT ATSEQLGKTA | 240 |
| GKDQAAAKAT YPSLLGLEAS RQKAEELIQS AKEALRPYGS QAEPLLALAD FITRRQH | 297 |

SEQ ID NO: 38

| | |
|---|---|
| atggtcgcac aaactttcaa cctggatacc tacttatccc aaagacaaca acaagttgaa | 60 |
| gaggccctaa gtgctgctct tgtgccagct tatcctgaga gaatatacga agctatgaga | 120 |
| tactccctcc tggcaggtgg caaaagatta agacctatct tatgtttagc tgcttgcgaa | 180 |
| ttggcaggtg gttctgttga acaagccatg ccaactgcgt gtgcacttga aatgatccat | 240 |
| acaatgtcac taattcatga tgacctgcca gccatggata acgatgattt cagaagagga | 300 |
| aagccaacta atcacaaggt gttcggggaa gatatagcca tcttagcggg tgatgcgctt | 360 |
| ttagcttacg cttttgaaca tattgcttct caaacaagag gagtaccacc tcaattggtg | 420 |
| ctacaagtta ttgctagaat cggacacgcc gttgctgcaa caggcctcgt tggaggccaa | 480 |
| gtcgtagacc ttgaatctga aggtaaagct atttccttag aaacattgga gtatattcac | 540 |
| tcacataaga ctggagcctt gctggaagca tcagttgtct caggcggtat tctcgcaggg | 600 |
| gcagatgaag agcttttggc cagattgtct cattacgcta gagatatagg cttggctttt | 660 |
| caaatcgtcg atgatatcct ggatgttact gctacatctg aacagttggg gaaaaccgct | 720 |
| ggtaaagacc aggcagccgc aaaggcaact tatccaagtc tattgggttt agaagcctct | 780 |
| agacagaaag cggaagagtt gattcaatct gctaaggaag ccttaagacc ttacggttca | 840 |
| caagcagagc cactcctagc gctggcagac ttcatcacac gtcgtcagca ttaa | 894 |

What is claimed is:

1. A method of producing an acetylated diterpene, comprising:
(a) providing a recombinant host cell capable of producing a diterpene, wherein the recombinant host cell comprises a recombinant gene encoding a diterpene acetyltransferase polypeptide capable of catalyzing acetylation of the diterpene;
and
(b) incubating the recombinant host cell under conditions in which the gene is expressed;
wherein the acetylated diterpene is produced by the recombinant host cell.

2. The method of claim 1, wherein the diterpene is 13R-manoyl oxide (13R-MO) or a 13R-MO derivative.

3. The method of claim 2, wherein the 13R-MO derivative is an oxidized 13R-MO derivative.

4. The method of claim 1, wherein the diterpene acetyltransferase polypeptide is a polypeptide having at least 55% identity to an amino acid sequence set forth in SEQ ID NO:6, SEQ ID NO:7, SEQ ID NO:8, SEQ ID NO:9, SEQ ID NO:10, SEQ ID NO:24, and/or SEQ ID NO:26.

5. The method of claim 1, wherein the acetylated diterpene is the acetylated diterpene of formula (I) 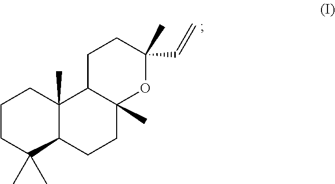 [SMILES: C=C[C@@]1(C)CCC2[C@@](C)(CC[C@H]3C(C)(C)CCC[C@]23C)O1]
where at least one hydrogen is replaced with an acetyl group; and
wherein the chemical valence requirement of the acetylated diterpene is satisfied.

6. The method of claim 5, wherein the acetylated diterpene is the acetylated diterpene of formula (I) substituted at one or more of the positions 1, 6, 7, 9, and/or 11 with an acetyl group.

7. The method of claim 1, wherein the acetylated diterpene is the acetylated diterpene of formula (I) 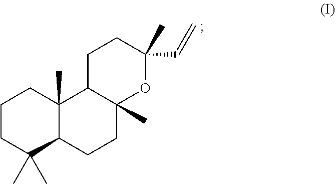 [SMILES: C=C[C@@]1(C)CCC2[C@@](C)(CC[C@H]3C(C)(C)CCC[C@]23C)O1]
where at least one hydrogen is replaced with an acetyl group;
wherein at least one of the other hydrogens is substituted with an —OH and/or ═O group; and
wherein the chemical valence requirement of the acetylated diterpene is satisfied.

8. The method of claim 7, wherein the acetylated diterpene is the acetylated diterpene of formula (I) substituted at two or more of the positions 1, 6, 7, 9, and/or 11;
wherein at least one position is substituted with an acetyl group; and
wherein at least one position is substituted with an —OH or ═O group.

9. The method of any one of claims 1-8, wherein the recombinant host cell is grown at a temperature for a period of time, wherein the temperature and period of time facilitate the production of the acetylated diterpene.

10. The method of claim 9, wherein the recombinant host cell is grown in a fermentor.

11. The method of any one of claims 1-10, that further comprises isolating the acetylated diterpene.

12. The method of any one of claims 1-11, wherein the acetylated diterpene is forskolin.

13. A recombinant host cell capable of producing an acetylated diterpene, wherein the recombinant host cell comprises a recombinant gene encoding a diterpene acetyltransferase polypeptide capable of catalyzing acetylation of the diterpene.

14. The method of claim 13, wherein the diterpene is 13R-MO or a 13R-MO derivative.

15. The method of claim 14, wherein the 13R-MO derivative is an oxidized 13R-MO derivative.

16. The recombinant host of claim 13, wherein the diterpene acetyltransferase polypeptide is a polypeptide having at least 55% identity to an amino acid sequence set forth in SEQ ID NO:6, SEQ ID NO:7, SEQ ID NO:8, SEQ ID NO:9, SEQ ID NO:10, SEQ ID NO:24, and/or SEQ ID NO:26.

17. The recombinant host cell of any one of claims 13-16, wherein the recombinant host cell further comprises:
(a) a gene encoding a diterpene synthase polypeptide of class I; and/or
(b) a gene encoding a diterpene synthase polypeptide of class II;
wherein at least one of these genes is a recombinant gene.

18. The recombinant host cell of any one of claims 13-16, wherein the recombinant host cell further comprises:
(a) a gene encoding a TPS2 polypeptide having at least 50% identity to an amino acid sequence set forth in SEQ ID NO:16;
(b) a gene encoding a TPS3 polypeptide having at least 50% identity to an amino acid sequence set forth in SEQ ID NO:17; and/or
(c) a gene encoding a TPS4 polypeptide having at least 40% identity to an amino acid sequence set forth in SEQ ID NO:18;
wherein at least one of these genes is a recombinant gene.

19. The recombinant host cell of any one of claims 13-16, wherein the recombinant host cell further comprises a recombinant gene encoding a polypeptide capable of catalyzing oxidation of 13R-MO.

20. The recombinant host cell of claim 19, wherein the gene encoding a polypeptide capable of catalyzing oxidation of 13R-MO comprises:
(a) a CYP76AH16 polypeptide having at least 55% identity to an amino acid sequence set forth in SEQ ID NO:19;
(b) a CYP76AH8 polypeptide having at least 50% identity to an amino acid sequence set forth in SEQ ID NO:20;
(c) a CYP76AH11 polypeptide having at least 50% identity to an amino acid sequence set forth in SEQ ID NO:21;
(d) a CYP76AH15 polypeptide having at least 50% identity to an amino acid sequence set forth in SEQ ID NO:22; and/or
(e) a CYP76AH17 polypeptide having at least 50% identity to an amino acid sequence set forth in SEQ ID NO:23.

21. The method of claim 1 or the recombinant host of claim 13, wherein the diterpene acetyltransferase polypeptide is a chimeric protein of one or more acetyltransferase polypeptides.

22. The method or recombinant host of claim 21, wherein the diterpene acetyltransferase polypeptide is ACT1-3A having an amino acid sequence set forth in SEQ ID NO:8, ACT1-3B having an amino acid sequence set forth in SEQ ID NO:24, and/or ACT1-4 having an amino acid sequence set forth in SEQ ID NO:9.

23. The recombinant host cell of any one of 13-22, wherein the recombinant host cell comprises a plant cell, a mammalian cell, an insect cell, a fungal cell, an algal cell or a bacterial cell.

24. The recombinant host cell of claim 23, wherein the bacterial cell comprises Escherichia cells, Lactobacillus cells, Lactococcus cells, Cornebacterium cells, Acetobacter cells, Acinetobacter cells, or Pseudomonas cells.

25. The recombinant host cell of claim 23, wherein the fungal cell comprises a yeast cell.

26. The recombinant host cell of claim 25, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

27. The recombinant host cell of claim 26, wherein the yeast cell is a Saccharomycete.

28. The recombinant host cell of claim 27, wherein the yeast cell is a Saccharomyces cerevisiae cell.

29. The recombinant host of claim 23, wherein the plant cell is a Nicotiana benthamiana cell.

30. The method of any one of claims 1-11, wherein the recombinant host cell is the recombinant host cell of any one of claims 13-29.

31. An acetylated diterpene composition produced by the method of any one of claim 1-11 or 21.

32. An acetylated diterpene composition produced by the recombinant host of any one of claims 13-29.

33. The acetylated diterpene composition of claim 31 or 32, wherein the acetylated diterpene composition is an acetylated 13R-MO composition.

34. The acetylated diterpene composition of claim 33, wherein the acetylated diterpene composition is a forskolin composition.
